(19) United States
(12) Patent Application Publication
(10) Pub. No.: US 2025/0049951 A1
(43) Pub. Date: Feb. 13, 2025

(54) NUCLEIC ACID COMPOSITIONS AND USES THEREOF

(71) Applicant: SURGE Therapeutics, Inc., Cambridge, MA (US)

(72) Inventor: Michael Solomon Goldberg, Brookline, MA (US)

(21) Appl. No.: 18/710,818

(22) PCT Filed: Nov. 16, 2022
(86) PCT No.: PCT/US22/50129

§ 371 Date: May 16, 2024

Related U.S. Application Data
(60) Provisional application No. 63/280,429, filed on Nov. 17, 2021.

(51) Int. Cl. A61K 48/00; A61K 38/16; A61K 38/20; A61K 47/34; A61K 49/00; A61P 35/00; G01N 33/50

(52) U.S. Cl. CPC A61K 48/0041; A61K 38/162; A61K 38/2086; A61K 47/34; A61K 49/0008; A61P 35/00; G01N 33/5011

(57) ABSTRACT

Provided are compositions useful for the treatment and/or prevention of cancer and metastatic tumors. For example, a provided composition comprises a biodegradable scaffold or biomaterial comprising one or more nucleic acid agents that may act to themselves promote or inhibit an immunomodulatory pathway, and/or may encode a peptide which may promote or inhibit an immunomodulatory pathway. Also provided are methods of making provided compositions as well as kits containing materials to provide the same.

### CROSS-REFERENCE TO RELATED APPLICATIONS

The present application claims the benefit of U.S. Provisional Application No. 63/280,429 filed on Nov. 17, 2021, the contents of which are hereby incorporated herein in their entireties.

### BACKGROUND

Systemic administration of medication, nutrition, or other substances into the circulatory system affects the entire body. Systemic routes of administration include enteral (e.g., oral dosage resulting in absorption of the drug through the gastrointestinal tract) and parenteral (e.g., intravenous, intramuscular, and subcutaneous injections) administration. Administration of immunotherapeutics typically relies on these systemic administration routes, which can lead to unwanted side effects. In some instances, certain promising therapeutics are extremely difficult to develop due to associated toxicities and the limitations of current administration methods and systems.

Surgery is often the first-line of treatment for solid tumor cancers and is generally used in combination with systemic administration of anti-cancer therapy. However, surgery-induced immunosuppression has been implicated in the development of post-operative septic complications and tumor metastasis due to changes in a variety of metabolic and endocrine responses, ultimately resulting in the death of many patients (Smyth, M. J. et al. Nature Reviews Clinical Oncology, 2016, 13, 143-158).

### SUMMARY

Systemic administration of immunotherapies can result in adverse side effects, e.g., inducing toxicities that are undesirable for non-cancerous cells and/or tissues such as non-tumor-specific immune cells, and/or requiring high doses in order to achieve sufficient concentration at a target site to induce a therapeutic response; and surgical resection of tumors can result in immunosuppression. Surgery can also induce cellular stress, which may involve, for example, activation of one or more physiological responses that promote wound healing after injury. Such responses include, e.g., activation of neural, inflammatory, and/or pro-angiogenic signaling pathways, which can also promote the growth and/or metastatic spread of cancer. Inflammatory changes that may occur at a surgical site following tumor resection can include, e.g., recruitment of immune and/or inflammatory cell type(s) and/or release of humoral factor(s). Local inflammatory wound response and systemic inflammation processes together might activate dormant micrometastases or induce the propagation of residual cancer cells, thus increasing the risk of cancer recurrence.

The present disclosure, provides, among other things, insights that include identification of the source of a problem with certain prior technologies including, for example, certain conventional approaches to cancer treatment. For example, the present disclosure appreciates that certain adverse events that can be associated with systemic administration of immunotherapeutic agents (e.g., skin rashes, hepatitis, diarrhea, colitis, hypophysitis, thyroiditis, and adrenal insufficiency) may be immune-related and may, at least in part, be attributable to exposure of non-tumor-specific immune cells to the systemically administered immunotherapeutic drug. Among other things, the present disclosure appreciates that the high doses typically required for systemic administration to achieve sufficient concentration in the tumor to induce a desired response may contribute to and/or be responsible for such undesirable effects. The present technology provides systems that solve such problems by providing, among other things, localized delivery of immunotherapeutic agents which, can improve efficacy by concentrating the action of the drug where it is needed. The present technology provides systems that solve such problems by providing localized delivery of immunotherapeutic agents comprising nucleic acids, or comprising products encoded by nucleic acids.

Moreover, the present disclosure provides insights that certain immunomodulatory agents traditionally used to treat autoimmune-type pathologies could be useful in the treatment of cancer if administered as described herein, notwithstanding that off-target toxicity would have otherwise been expected to be in opposition to those anticipated for an anti-cancer immunomodulatory compound. Furthermore, the present disclosure recognizes that certain immunomodulatory agents may be nucleic acids, or may be products encoded by nucleic acids. Thus, the present disclosure teaches usefulness for cancer therapy of agents previously not considered useful and furthermore teaches delivery and dosing strategies that are particularly effective and/or desirable for these and other agents.

In one aspect, the present disclosure provides a method comprising a step of: administering to a target site in a tumor resection subject, a composition comprising: (i) a polymeric biomaterial; and (ii) a polynucleotide agent that encodes or regulates an immunomodulatory polypeptide. In some embodiments, a polynucleotide agent encodes a cytokine that induces innate immunity and/or adaptive immunity. In some, a polynucleotide agent activates a pattern recognition receptor that induces innate immunity. In some embodiments, a polynucleotide agent encodes a chemokine that induces recruitment of immune cells. In some embodiments, a polynucleotide agent encodes an antibody that mediates immune checkpoint blockade or co-stimulation.

In some embodiments, a target site is a tumor resection site. In some embodiments, a target site is a site near a tumor resection site. In some embodiments, a target site is a sentinel lymph node. In some embodiments, a step of administering is by injection.

In some embodiments, a composition is liquid and a polymeric biomaterial is a viscous polymer solution. In some embodiments, a composition is liquid and a polymeric biomaterial, upon administration, forms a polymer network biomaterial in situ at a target site. In some embodiments, a polymer network biomaterial includes or is a crosslinked polymer network biomaterial. In some embodiments, a polymer network biomaterial includes or is a non-crosslinked polymer network biomaterial.

In some embodiments, a polymeric biomaterial is characterized in that when tested in vitro by placing a combination of the polymeric biomaterial and a polynucleotide agent in PBS (pH 7.4), less than 100% of the polynucleotide agent is released within 3 hours from the polymeric biomaterial. In some embodiments, a polymeric biomaterial is characterized in that when tested in vivo by administering a combination of the polymeric biomaterial and a polynucleotide agent to a mammary fat pad of a mouse subject, less than or equal to 50% of the polynucleotide agent is released in vivo 8 hours after administration. In some embodiments, a polymeric biomaterial is characterized in that when tested in vivo by administering a combination of the polymeric biomaterial and a polynucleotide agent to a mammary fat pad of a mouse subject, the polymeric biomaterial extends release of the polynucleotide agent such that, when assessed at 24 hours after administration, more polynucleotide agent is present at the mammary fat pad than is observed when the polynucleotide agent is administered in solution. In some embodiments, a polymeric biomaterial is characterized in that a polynucleotide agent is released from the polymeric biomaterial and is taken up by local cells so that (i) at least a subset of local immune cells express a immunomodulatory polypeptide encoded by the polynucleotide agent, (ii) at least a subset of local immune cells have an increased expression of a type 1 interferon in response to innate immune stimulation induced by the polynucleotide agent, and/or (iii) at least a subset of local immune cells have a change in level and/or activity of a immunomodulatory polypeptide.

In some embodiments, a polymeric biomaterial is characterized by a storage modulus of about 10 Pa to about 5,000 Pa. In some embodiments, a polymeric biomaterial includes or is a hydrogel. In some embodiments, a polymeric biomaterial includes a positively-charged polymer.

In some embodiments, a polymeric biomaterial further includes a polynucleotide agent carrier. In some embodiments, a polynucleotide agent is complexed to a polynucleotide agent carrier. In some embodiments, a polynucleotide agent is loaded in a polynucleotide agent carrier. In some embodiments, a polynucleotide agent carrier includes a cationic agent and/or a lipid.

In some embodiments, a step of administering does not involve administration of a tumor antigen to a tumor resection subject. In some embodiments, a step of administering does not involve administration of a microparticle to a tumor resection subject. In some embodiments, a step of administering does not involve adoptive transfer of immune cells to a tumor resection subject.

In some embodiments, a composition further includes an inhibitor of proinflammatory immune response mediated by p38 mitogen-activated protein kinase (MAPK) pathway. In some embodiments, a composition further includes an activator of innate immunity. In some embodiments, an activator of innate immunity is or includes a stimulator of interferon genes (STING) agonist. In some embodiments, an activator of innate immunity is or includes a Toll-like receptor (TLR) 7 and/or TLR8 (“TLR7/8”) agonist. In some embodiments, a composition further includes an activator of adaptive immunity.

In some embodiments, a polymeric biomaterial forms a matrix or depot and a polynucleotide agent is within the polymeric biomaterial. In some embodiments, a polynucleotide agent is released by diffusion through a polymeric biomaterial. In some embodiments, a polymeric biomaterial is biodegradable in vivo.

In some embodiments, a tumor resection site is characterized by absence of gross residual tumor antigen. In some embodiments, a tumor resection subject is suffering from metastatic cancer. In some embodiments, a method further includes a step of monitoring at least one metastatic site in a tumor resection subject after administration to a target site in a tumor resection subject, a composition including: (i) a polymeric biomaterial; and (ii) a polynucleotide agent that encodes or regulates an immunomodulatory polypeptide.

In one aspect, the present disclosure provides a method of generating a polymer network biomaterial composition, the method including steps of: (a) providing a composition including one or more precursor components of a polymer network biomaterial and a polynucleotide agent that encodes or regulates an immunomodulatory polypeptide; and (b) allowing the precursor components to form the polymer network biomaterial in less than 10 minutes, where the polymer network biomaterial is characterized in that: the polymer network biomaterial has a storage modulus of less than 5,000 Pa; and/or when tested in vitro by placing a combination of the polymer network biomaterial and the polynucleotide agent in PBS (pH 7.4), less than 100% of the polynucleotide agent is released within 3 hours from the polymer network biomaterial; when tested in vivo by administering a combination of the polymer network biomaterial and the polynucleotide agent to a mammary fat pad of a mouse subject, less than or equal to 50% of the polynucleotide agent is released in vivo 8 hours after administration; when tested in vivo by administering a combination of the polymer network biomaterial and the polynucleotide agent to a mammary fat pad of a mouse subject, the polymer network biomaterial extends release of the polynucleotide agent such that, when assessed at 24 hours after administration, more polynucleotide agent is present at the mammary fat pad than is observed when the polynucleotide agent is administered in solution; and/or the polynucleotide agent released from the polymer network biomaterial is taken up by local cells so that (i) at least a subset of local immune cells express the immunomodulatory polypeptide encoded by the polynucleotide agent, (ii) at least a subset of local immune cells have an increased expression of a type 1 interferon in response to innate immune stimulation induced by the polynucleotide agent, and/or (iii) at least a subset of local immune cells have a change in level and/or activity of the immunomodulatory polypeptide.

In some embodiments, a polymer network biomaterial includes or is a crosslinked polymer network biomaterial. In some embodiments, a polymer network biomaterial includes or is a non-crosslinked polymer network biomaterial. In some embodiments, a polymer network biomaterial includes or is a hydrogel. In some embodiments, a polymer network biomaterial includes a polynucleotide agent carrier. In some embodiments, a polynucleotide agent is complexed to a polynucleotide agent carrier. In some embodiments, a polynucleotide agent is loaded in a polynucleotide agent carrier. In some embodiments, a polynucleotide agent carrier includes a cationic agent and/or a lipid.

In one aspect, the present disclosure provides a method of characterizing a polymer network biomaterial composition or component(s) thereof, the method including steps of: (a) providing the polymer network biomaterial composition in a buffered solution in vitro, where the polymer network biomaterial composition includes a candidate polymeric biomaterial and a polynucleotide agent that encodes or regulates an immunomodulatory polypeptide; and (b) determining whether less than 100% of the polynucleotide agent is released within 3 hours from the polymeric biomaterial. In some embodiments, a buffered solution includes or is PBS (pH 7.4). In some embodiments, a step of determining further includes measuring amounts of a polynucleotide agent released from a polymeric biomaterial at pre-determined time points over a period of at least 3 hours. In some embodiments, a step of determining further includes determining the release profile kinetics of a polynucleotide agent from a polymeric biomaterial. In some embodiments, a method further includes selecting a polymer network biomaterial composition characterized in that less than 100% of a polynucleotide agent is released within 3 hours from a polymeric biomaterial.

In some embodiments, a method further includes contacting a selected polymer network biomaterial composition with a population of cells, and determining: (a) whether a polynucleotide agent released from a candidate polymeric biomaterial is taken up by immune cells; and/or (b) whether immune cells that take up a polynucleotide agent exhibits at least one of a following biological activities: (i) expressing an immunomodulatory polypeptide encoded by the polynucleotide agent, (ii) exhibiting an increased expression of a type 1 interferon in response to innate immune stimulation induced by the polynucleotide agent, and/or (iii) exhibiting a change in level and/or activity of a immunomodulatory polypeptide. In some embodiments, an immune cells include myeloid cells and/or plasmacytoid dendritic cells. In some embodiments, cells further include non-immune cells. In some embodiments, non-immune cells include fibroblasts and/or endothelial cells. In some embodiments, a method further includes determining: (a) whether a polynucleotide agent released from a candidate polymeric biomaterial is taken up by non-immune cells; and/or (b) whether a polynucleotide agent exerts an adverse effect on non-immune cells.

In one aspect, the present disclosure provides a method of characterizing a polymer network biomaterial composition or component(s) thereof, the method including steps of: (a) administering a polymer network composition to a target site of a mouse subject in vivo, where the polymer network biomaterial composition includes a candidate polymeric biomaterial and a polynucleotide agent that encodes or regulates an immunomodulatory polypeptide; and (b) determining: (i) whether the candidate polymeric biomaterial extends release of the polynucleotide agent at a target site, upon administration, by at least 24 hours relative to administration of the same polynucleotide agent in solution without the polymeric biomaterial; and/or (ii) whether a release profile of the polynucleotide agent from the candidate polymeric biomaterial is characterized in that: less than or equal to 50% of the polynucleotide agent is released in vivo 8 hours after administration. In some embodiments, a target site is a mammary fat pad. sIn some embodiments, a method further includes selecting a polymer network biomaterial composition characterized in that (i) a candidate polymeric biomaterial extends release of a polynucleotide agent at a target site, upon administration, by at least 24 hours relative to administration of the same polynucleotide agent in solution without the polymeric biomaterial; and/or (ii) less than or equal to 50% of the polynucleotide agent is released from the candidate polymeric biomaterial in vivo 8 hours after administration.

In some embodiments, a method further includes determining: (a) whether a polynucleotide agent released from a candidate polymeric biomaterial is taken up by immune cells; and/or (b) whether immune cells that take up the polynucleotide agent exhibits at least one of the following biological activities: (i) expressing an immunomodulatory polypeptide encoded by the polynucleotide agent, (ii) exhibiting an increased expression of a type 1 interferon in response to innate immune stimulation induced by the polynucleotide agent, and/or (ii) exhibiting a change in level and/or activity of an immunomodulatory polypeptide. In some embodiments, a method further includes determining: (c) whether cellular uptake of a polynucleotide agent released from a candidate polymeric biomaterial is delayed, as compared to that when cells are contacted with the polynucleotide agent in the absence of the candidate polymeric biomaterial; and/or (d) whether at least one of a biological activities induced by the polynucleotide agent is delayed, as compared to that when cells are contacted with the polynucleotide agent in the absence of the candidate polymeric biomaterial. In some embodiments, immune cells include myeloid cells and/or plasmacytoid dendritic cells. In some embodiments, a method further includes determining: (e) whether a polynucleotide agent released from a candidate polymeric biomaterial is taken up by non-immune cells; and/or (f) whether a polynucleotide agent exerts an adverse effect on non-immune cells. In some embodiments, non-immune cells include fibroblasts and/or endothelial cells.

In one aspect, the present disclosure provides a method including a step of: administering to a target site in a tumor resection subject, a composition including: (i) a polynucleotide agent carrier; and (ii) a polynucleotide agent that encodes or regulates an immunomodulatory polypeptide. In some embodiments, a polynucleotide agent carrier includes a cationic agent and/or a lipid.

### BRIEF DESCRIPTION OF THE DRAWINGS

FIG. 1 is a graphical representation of a survival analysis. The data is presented as a Kaplan-Meier survival curve, showing the survival of female BALB/cJ mice inoculated orthotopically with 4T1-Luc2 cells. These cells were allowed to create tumors, and those tumors were subsequently surgically resected. Immediately following tumor resection, an exemplary composition comprising a polymer network (a crosslinked hyaluronic acid hydrogel) and an innate immune system agonist, (RIG-1 agonist) was implanted at the tumor resection site. Administration of localized compositions facilitated sustained release of the active agent (RIG-1 agonist), resulting in significantly increased lifespan of animals when compared to control implantations lacking an active agent.

FIG. 2 is a graphical representation of survival analysis. The data is presented as a Kaplan-Meier survival curve, showing the survival of female BALB/cJ mice inoculated orthotopically with 4T1-Luc2 cells. These cells were allowed to create tumors, and those tumors were subsequently surgically resected. Immediately following tumor resection, an exemplary composition comprising a polymer network (a poloxamer and hyaluronic acid hydrogel) and an innate immune system agonist (RIG-1 agonist) was injected at the tumor resection site. Administration of localized compositions facilitated sustained release of the active agent (RIG-1 agonist), resulting in increased lifespan of animals when compared to control implantations lacking an active agent.

FIG. 3 is a graphical representation of a survival analysis. The data is presented as a Kaplan-Meier survival curve, showing the survival of female BALB/cJ mice inoculated orthotopically with 4T1-Luc2 cells. These cells were allowed to create tumors, and those tumors were subsequently surgically resected. Immediately following tumor resection, an exemplary composition comprising a polymer network (a crosslinked hyaluronic acid hydrogel) and a nucleic acid encoding an innate immune system agonist, (Interleukin-15 (IL-15) mRNA) was implanted at the tumor resection site. Administration of localized compositions facilitated sustained release of the active agent (IL-15 mRNA), resulting in increased lifespan of animals when compared to control implantations lacking an active agent.

FIG. 4 is a graphical representation of a survival analysis. The data is presented as a Kaplan-Meier survival curve, showing the survival of female BALB/cJ mice inoculated orthotopically with 4T1-Luc2 cells. These cells were allowed to create tumors, and those tumors were subsequently surgically resected. Immediately following tumor resection, an exemplary composition comprising a polymer network (a poloxamer hydrogel or a poloxamer and hyaluronic acid hydrogel) and a nucleic acid encoding an innate immune system agonist (Interleukin-15 (IL-15) mRNA) was injected at the tumor resection site. Administration of localized compositions containing hyaluronic acid resulted in increased lifespan of animals when compared to compositions lacking hyaluronic acid.

### CERTAIN DEFINITIONS

It is noted that the concentrations of individual polymer components in polymeric biomaterial(s) preparations described herein are each expressed in % (w/w) or wt %. As used herein, the concentration, % (w/w), of a polymer component in a polymeric biomaterial(s) preparation is determined based on the mass or weight of the polymer component relative to the sum of (i) total mass or weight of all individual polymer components present in the polymeric biomaterial(s) preparation and (ii) total mass or weight solvent used in the polymeric biomaterial(s) preparation.

Activator of adaptive immune response: The term “activator of adaptive immune response” refers to an agent that activates (e.g., increases the activity of) an adaptive immune system (and/or one or more features of an adaptive immune system) in a subject (e.g., in a subject to whom it is administered and/or who is otherwise in need thereof), as compared to when the agent is absent. Such activation can restore or enhance antitumor function, for example, by neutralizing inhibitory immune checkpoints and/or by triggering co-stimulatory receptors, ultimately generating helper and/or effector T cell responses against immunogenic antigens expressed by cancer cells and producing memory B cell, and/or T cell populations. In certain embodiments, an activator of adaptive immune response involves modulation of an adaptive immune response and/or leukocyte trafficking. Examples of activators of adaptive immune response include, e.g., ones described in WO 2018/045058, the contents of which are incorporated herein by reference in their entirety for the purposes described herein.

Activator of innate immune response: The term “activator of innate immune response” refers to an agent that activates (e.g., increases the activity of) an innate immune system (and/or one or more features of an innate immune system) in a subject (e.g., in a subject to whom it is administered and/or who is otherwise in need thereof), as compared to when the agent is absent. Such activation can stimulate (e.g., can increase expression level and/or activity of) one or more agents that initiate an inflammatory response (e.g., an immunostimulatory inflammatory response) and/or help to induce adaptive immune responses, for example, leading to the development of antigen-specific acquired immunity. In some embodiments, activation of the innate immune system can lead to recruitment of relevant immune cells including, e.g., but not limited to neutrophils, basophils, eosinophils, natural killer cells, dendritic cells, monocytes, and macrophages, cytokine production, leukocyte proliferation and/or survival, as well as improved T cell priming, for example by augmenting presentation of antigens and/or expression level and/or activity of co-stimulatory molecules by antigen-presenting cells. Examples of activators of innate immune response include, e.g., ones described in WO 2018/045058, the contents of which are incorporated herein by reference in their entirety for the purposes described herein.

Administer: As used herein, the term “administer,” “administering,” or “administration” typically refers to the administration of a composition to a subject to achieve delivery of an agent or payload that is, or is included in, a composition to a target site or a site to be treated. Those of ordinary skill in the art will be aware of a variety of routes that may, in appropriate circumstances, be utilized for administration of different agents to a subject, for example a human. For example, while the terms “administer,” “administering,” or “administration” refer to implanting, absorbing, ingesting, injecting, inhaling, parenteral administration, or otherwise introducing a composition as described herein, in the context of administering a composition comprising a composition described herein, administering may refer to, in some embodiments, implanting, or in some embodiments, injecting.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Agonist: Those skilled in the art will appreciate that the term “agonist” may be used to refer to an agent, condition, or event whose presence, level, degree, type, or form correlates with increased level and/or activity of another agent (i.e., the agonized agent) and/or an increase in or induction of one or more biological events. In general, an agonist may be or include an agent of various chemical class including, for example, small molecules, polypeptides, nucleic acids, carbohydrates, lipids, metals, inorganic crystals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

Antagonist: Those skilled in the art will appreciate that the term “antagonist” may refer to an agent, condition, or event whose presence, level, degree, type, or form is associated with a decreased level and/or activity of another agent (i.e., the antagonized agent) and/or a decrease in or suppression of one or more biological events. In general, an antagonist may include an agent of various chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3—, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi-or multi-specific antibodies (e.g., Zybodies®, etc); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)₂, Fab′, di-sdFv, single domain antibodies, trifunctional antibodies, diabodies, and minibodies, etc. In some embodiments, relevant formats may be or include: Adnectins®; Affibodies®; Affilins®; Anticalins®; Avimers®; BiTERs; cameloid antibodies; Centyrins®; ankyrin repeat proteins or DARPINs®; dual-affinity re-targeting (DART) agents; Fynomers®; shark single domain antibodies such as IgNAR; immune mobilizing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Bioadhesive: The term “bioadhesive” refers to a biocompatible agent that can adhere to a target surface, e.g., a tissue surface. In some embodiments, a bioadhesive can adhere to a target surface, e.g., a tissue surface, and retain on the target surface, e.g., for a period of time. In some embodiments, a bioadhesive may be biodegradable. In some embodiments, a bioadhesive may be a natural agent, which may have been prepared or obtained, for example, by isolation or by synthesis; in some embodiments, a bioadhesive may be a non-natural agent, e.g., as may have been designed and/or manufactured by the hand of man (e.g., by processing, synthetic, and/or recombinant production, depending on the agent, as will be understood by those skilled in the art. In some particular embodiments, a bioadhesive may be or comprise a polymeric material, e.g., as may be comprised of or contain a plurality of monomers such as sugars. Certain exemplary bioadhesives include a variety of FDA-approved agents such as, for example, cyanoacrylates (Dermabond, 2-Octyl cyanoacrylate; Indermil, n-Butyl-2-cyanoacrylate; Histoacryl and Histoacryl Blue, n-Butyl-2-cyanoacrylate), albumin and glutaraldehyde (BioGlue™, bovine serum albumin and 10% glutaraldehyde), fibrin glue (Tisseel™, human pooled plasma fibrinogen and thrombin; Evicel™, human pooled plasma fibrinogen and thrombin; Vitagel™, autologous plasma fibrinogen and thrombin; Cryoseal™ system, autologous plasma fibrinogen and thrombin), gelatin and/or resorcinol crosslinked by formaldehyde and/or glutaraldehyde, polysaccharide-based adhesives (e.g., alginate, chitosan, collagen, dextran, and/or gelatin), PEG, acrylates, polyamines, or urethane variants (isocyanate-terminated prepolymer, and/or combinations thereof. Other examples of bioadhesives that are known in the art, e.g., as described in Mehdizadeh and Yang “Design Strategies and Applications of Tissue Bioadhesives” Macromol Biosci 13:271-288 (2013), can be used for the purposes of the methods described herein. In some embodiments, a bioadhesive can be a degradable bioadhesive. Examples of such a degradable bioadhesive include, but are not limited to fibrin glues, gelatin-resorcinol-formaldehyde/glutaraldehyde glues, poly(ethylene glycol) (PEG)-based hydrogel adhesives, polysaccharide adhesives, polypeptide adhesives, polymeric adhesives, biomimetic bioadhesives, and ones described in Bhagat and Becker “Degradable Adhesives for Surgery and Tissue Engineering” Biomacromolecules 18:3009-3039 (2017).

Biocompatible: The term “biocompatible”, as used herein, refers to materials that do not cause significant harm to living tissue when placed in contact with such tissue, e.g., in vivo. Biocompatibility of a material can be gauged by the ability of such a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, and/or immunogenicity. In certain embodiments, materials are “biocompatible” if they themselves are not toxic to cells in an in vivo environment of its intended use. In certain embodiments, materials are “biocompatible” if their addition to cells in vitro results in less than or equal to 20% cell death and/or their administration in vivo does not induce significantly severe inflammation that is clinically undesirable for purposes described herein or other such adverse effects. As will be understood by those skilled in the art that such significantly severe inflammation is distinguishable from mild, transient inflammation, which typically accompanies surgery or introduction of foreign objects into a living organism. Furthermore, one of skill in the art will appreciate, reading the present disclosure, that in some embodiments, polymeric biomaterial(s) preparations described herein and/or individual polymer components thereof are biocompatible if extent of immunomodulation (e.g., innate immunity agonism) over a defined period of time is clinically beneficial and/or desirable, e.g., to provide antitumor immunity.

Biologic: The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

Biological sample: The term “biological sample” refers to a primary sample obtained from a biological source and/or, in some embodiments, to a sample derived therefrom (e.g., by processing). Those skilled in the art appreciate that biological samples may include or be selected from, for example, tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

Cancer: The term “cancer” refers to a malignant neoplasm (Stedman's Medical Dictionary, 25th ed.; Hensyl ed.; Williams & Wilkins: Philadelphia, 1990). Of particular interest in the context of some embodiments of the present disclosure are cancers treated by cell killing and/or removal therapies (e.g., surgical resection and/or certain chemotherapeutic therapies such as cytotoxic therapies, etc). In some embodiments, a cancer that is treated in accordance with the present disclosure is one that has been surgically resected (i.e., for which at least one tumor has been surgically resected). In some embodiments, a cancer that is treated in accordance with the present disclosure is one for which resection is standard of care. In some embodiments, a cancer that is treated in accordance with the present disclosure is one that has metastasized. In certain embodiments, exemplary cancers may include one or more of acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget's disease of the vulva).

Carbohydrate polymer: The term “carbohydrate polymer” refers to a polymer that is or comprises one or more carbohydrates, e.g., having a carbohydrate backbone. For example, in some embodiments, a carbohydrate polymer refers to a polysaccharide or an oligosaccharide, or a polymer containing a plurality of monosaccharide units connected by covalent bonds. The monosaccharide units may all be identical, or, in some cases, there may be more than one type of monosaccharide unit present within the carbohydrate polymer. In certain embodiments, a carbohydrate polymer is naturally occurring. In certain embodiments, a carbohydrate polymer is synthetic (i.e., not naturally occurring). In some embodiments, a carbohydrate polymer may comprise a chemical modification. In some embodiments, a carbohydrate polymer is a linear polymer. In some embodiments, a carbohydrate polymer is a branched polymer.

Chemotherapeutic agent: The term “chemotherapeutic agent” refers to a therapeutic agent known to be of use in chemotherapy for cancer. For example, in some embodiments, a chemotherapeutic agent can inhibit the proliferation of rapidly growing cancer cells and/or kill cancer cells. Examples of such chemotherapeutic agents include, but are not limited to alkylating agents, anti-metabolites, topoisomerase inhibitors, and/or mitotic inhibitors.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Colloid: As used herein, the term “colloid” refers to a homogenous solution or suspension of particles (e.g., polymer particles) dispersed though a continuous medium (e.g., an aqueous buffer system). In some embodiments, a colloid is an emulsion. In some embodiments, a colloid is a sol. In some embodiments, a colloid is a gel.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those of ordinary skill in the art will also understand that when the term “comparable” is used in the context of comparison of two or more values, such values are comparable to one another such that the differences in values do not result in material differences in therapeutic outcomes, e.g., induction of anti-tumor immunity and/or incidence of tumor regrowth and/or metastasis. For example, in some embodiments, comparable release rates refer to values of such release rates within 15% over a period of 48 hours. In some embodiments, comparable release rates refer to values of such release rates within 20% over a period of 48 hours. In some embodiments, comparable release rates refer to values of such release rates within 15% over a period of 24 hours.

Condition, disease, or disorder: The terms “condition,” “disease,” and “disorder” are used interchangeably.

Critical gelation temperature: As used herein, the term “critical gelation temperature”, abbreviated as “CGT”, refers to a threshold temperature at or above which a precursor state of a biomaterial preparation (e.g., ones described herein) transitions to a polymer network state described herein (e.g., a hydrogel state). In some embodiments, a critical gelation temperature may correspond to a sol-gel transition temperature. In some embodiments, a critical gelation temperature may correspond to a lower critical solution temperature. See Taylor et al., “Thermoresponsive Gels” Gels (2017) 3:4, for general description of thermoresponsive gels, the contents of which are incorporated herein by reference for purposes described herein. As described in the present disclosure, certain embodiments of biomaterial preparations described herein are demonstrated to form a polymer network state when it is exposed to a temperature of about 35-40° C. One of ordinary skill in the art, reading the present disclosure, will understand that such biomaterial preparations do not necessarily have a CGT of about 35-40° C., but may rather have a CGT that is lower than 35-40° C. For example, in some embodiments, provided biomaterial preparations may have a CGT of about 20-28° C.

Crosslink: As used herein, the term “crosslink” refers to interaction and/or linkage between one entity and another entity to form a network. For example, in some embodiments, crosslinks present in polymer network may be or comprise intra-molecular crosslinks, inter-molecular crosslinks, or both. In some embodiments, crosslinks may comprise interactions and/or linkages between one polymer chain(s) and another polymer chain(s) to form a polymer network. In some embodiments, a crosslink may be achieved using one or more physical crosslinking approaches, including, e.g., one or more environmental triggers and/or physiochemical interactions. Examples of an environmental trigger include, but are not limited to pH, temperature, and/or ionic strength. Non-limiting examples of physiochemical interactions include hydrophobic interactions, charge interactions, hydrogen bonding interactions, stereocomplexation, and/or supramolecular chemistry. In some embodiments, a crosslink may be achieved using one or more covalent crosslinking approaches (e.g., where the linkage between two entities is or comprises a covalent bond) based on chemistry reactions, e.g., in some embodiments which may include reaction of an aldehyde and an amine to form a Schiff base, reaction of an aldehyde and hydrazide to form a hydrazine, and/or Michael reaction of an acrylate and either a primary amine or a thiol to form a secondary amine or a sulfide. Examples of such covalent crosslinking approaches include, but are not limited to small-molecule crosslinking and polymer-polymer crosslinking. Various methods for physical and covalent crosslinking of polymer chains are known in the art, for example, as described in Hoare and Kohane, “Hydrogels in drug delivery: Progress and challenges” Polymer (2008) 49:1993-2007, the entire content of which is incorporated herein by reference for the purposes disclosed herein.

Crosslinker: As used interchangeably herein, the term “crosslinker” or “crosslinking agent” refers to an agent that links one entity (e.g., one polymer chain) to another entity (e.g., another polymer chain). In some embodiments, linkage (i.e., the “crosslink”) between two entities is or comprises a covalent bond. In some embodiments, linkage between two entities is or comprises an ionic bond or interaction. In some embodiments, a crosslinker is a chemical crosslinker, which, e.g., in some embodiments may be or comprise a small molecule (e.g., dialdehydes or genipin) for inducing formation of a covalent bond between an aldehyde and an amino group. In some embodiments, a crosslinker comprises a photo-sensitive functional group. In some embodiments, a crosslinker comprises a pH-sensitive functional group. In some embodiments, a crosslinker comprises a thermal-sensitive functional group.

Disease: As used herein, the term “disease” refers to a disorder or condition that typically impairs normal functioning of a tissue or system in a subject (e.g., a human subject) and is typically manifested by characteristic signs and/or symptoms. Examples of diseases that are amenable for technologies provided herein include, but are not limited to autoimmune diseases, inflammatory diseases, bone diseases, metabolic diseases, neurological and neurodegenerative diseases, cancer, cardiovascular diseases, allergies and asthma, Alzheimer's disease, and hormone-related diseases. In some embodiments, a disease amenable to technologies provided herein is cancer.

Effective amount: An “effective amount” is an amount sufficient to elicit a desired biological response, e.g., treating a condition from which a subject may be suffering. As will be appreciated by those of ordinary skill in this art, the effective amount of a composition or an agent included in the composition may vary depending on such factors as the desired biological endpoint, the physical, chemical, and/or biological characteristics (e.g., pharmacokinetics and/or degradation) of agents in the composition, the condition being treated, and the age and health of the subject. In some embodiments, an amount may be effective for therapeutic treatment; alternatively or additionally, in some embodiments, an amount may be effective for prophylactic treatment. For example, in treating cancer, an effective amount may prevent tumor regrowth, reduce the tumor burden, or stop the growth or spread of a tumor. Those skilled in the art will appreciate that an effective amount need not be contained in a single dosage form. Rather, administration of an effective amount may involve administration of a plurality of doses, potentially over time (e.g., according to a dosing regimen). For example, in some embodiments, an effective amount may be an amount administered in a dosing regimen that has been established, when administered to a relevant population, to achieve a particular result with statistical significance.

Hydrate: The term “hydrate”, as used herein, has its art-understood meaning and refers to an aggregate of a compound (which may, for example be a salt form of the compound) and one or more water molecules. Typically, the number of the water molecules contained in a hydrate of a compound is in a definite ratio to the number of the compound molecules in the hydrate. Therefore, a hydrate of a compound may be represented, for example, by the general formula Rx×H2O, wherein R is the compound and x is a number greater than 0. A given compound may form more than one type of hydrate, including, e.g., monohydrates (x is 1), lower hydrates (x is a number greater than 0 and smaller than 1, e.g., hemihydrates (R×0.5 H2O)), and polyhydrates (x is a number greater than 1, e.g., dihydrates (R×2 H2O) and hexahydrates (R×6 H2O)).

Hydrogel: The term “hydrogel” has its art-understood meaning and refers to a material formed from a network of polymer chains that are hydrophilic, sometimes found as a colloidal gel in which an aqueous phase is the dispersion medium. In some embodiments, hydrogels are highly absorbent (e.g., they can absorb and/or retain over 90% water) natural or synthetic polymeric networks. In some embodiments, hydrogels possess a degree of flexibility similar to natural tissue, for example due to their significant water content.

Immunotherapy: The term “immunotherapy” refers to a therapeutic agent that promotes the treatment of a disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, nucleic acids, small molecule inhibitors, and small molecule agonists. For example, useful immunotherapies may include, but are not limited to, inducers of type I interferon, interferons, stimulator of interferon genes (STING) agonists, TLR7/8 agonists, IL-15 superagonists, COX inhibitors (e.g., COX-1 inhibitors and/or COX-2 inhibitors), anti-PD-1 antibodies, anti-CD137 antibodies, and anti-CTLA-4 antibodies. In some embodiments, certain polymeric biomaterial(s) preparations provided herein are themselves immunomodulatory (e.g., sufficient to induce anti-tumor immunity) in the absence of immunotherapy and thus do not include administration of such immunotherapy as described herein.

Immunomodulatory payload: As used herein, the term “immunomodulatory payload” refers to a separate immunomodulatory agent (e.g., small molecules, polypeptides (including, e.g., cytokines), nucleic acids, etc.) that can be carried by or distributed in a polymeric biomaterial(s) preparation such as ones as provided and/or utilized herein), wherein the immunomodulatory agent provides a therapeutic effect of modulating or altering (e.g., inducing, enhancing, or suppressing, etc.) one or more aspects of an immune response in a subject. Examples of an immunomodulatory payload include, but are not limited to activators of adaptive immune response, activators of innate immune response, inhibitors of a proinflammatory pathway, immunomodulatory cytokines, or immunomodulatory therapeutic agents as well as ones as described in WO 2018/045058 and WO 2019/183216, and any combinations thereof. The contents of the aforementioned patent applications are incorporated herein by reference for the purposes described herein. In some embodiments, an immunomodulatory payload is or comprises an innate immunity modulatory payload (e.g., an immunomodulatory payload that induces or stimulates innate immunity and/or one or more features of innate immunity). In some embodiments, an innate immunity modulatory payload is or comprises an activator of innate immune response. In some embodiments, an immunomodulatory payload is or comprises an adaptive immunity modulatory payload, e.g., an activator of adaptive immune response. In some embodiments, an immunomodulatory payload is or comprises an inhibitor of a proinflammatory pathway, e.g., an inhibitor of proinflammatory immune response mediated by a p38 mitogen-activated protein kinase (MAPK) pathway. In some embodiments, an immunomodulatory payload is or comprises an immunomodulatory cytokine. In some embodiments, an immunomodulatory payload is or comprises an immunomodulatory therapeutic agent. As will be understood by those skilled in the art, an immunomodulatory payload does not include components (e.g., precursor components) and/or by-products of a polymeric biomaterial(s) preparation (e.g., as described and/or utilized herein) generated, e.g., by chemical, enzymatic, and/or biological reactions such as, e.g., degradation.

Implanting: The terms “implantable,” “implantation,” “implanting,” and “implant” refer to positioning a composition of interest at a specific location in a subject, such as within a tumor resection site or in a sentinel lymph node, and typically by general surgical methods.

Increased, Induced, or Reduced: As used herein, these terms or grammatically comparable comparative terms, indicate values that are relative to a comparable reference measurement. For example, an assessed value achieved in a subject may be “increased” relative to that obtained in the same subject under different conditions (e.g., prior to or after an event; or presence or absence of an event such as administration of a composition or preparation as described and/or utilized herein, or in a different, comparable subject (e.g., in a comparable subject that differs from the subject of interest in prior exposure to a condition, e.g., absence of administration of a composition or preparation as described and/or utilized herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Inhibit: The term “inhibit” or “inhibition” is not limited to only total inhibition. Thus, in some embodiments, partial inhibition or relative reduction is included within the scope of the term “inhibition.” For example, in the context of modulating level (e.g., expression and/or activity) of a target, the term, in some embodiments, refers to a reduction in the level (e.g., expression and/or activity) of a target to a level that is reproducibly and/or statistically significantly lower than an initial or other appropriate reference level, which may, for example, be a baseline level of a target. In some embodiments, the term refers to a reduction in the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target. In the context of risk and/or incidence of tumor recurrence and/or metastasis, the term, in some embodiments, refers to a reduction of the risk or incidence of tumor recurrence and/or metastasis to a level that is reproducibly and/or statistically significantly lower than an initial or other appropriate reference level, which may, for example, be a baseline level of risk or incidence of tumor recurrence and/or metastasis in the absence or prior to administration of a composition described herein. In some embodiments, the term refers to a reduction of the risk or incidence of tumor recurrence and/or metastasis to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of risk or incidence of tumor recurrence and/or metastasis in the absence or prior to administration of a composition described herein. In the context of modulation of an immune cell function (e.g., by inhibiting activity and/or expression of a target), the term, in some embodiments, refers to a reduction of the activity and/or expression of a target to a level that is reproducibly and/or statistically significantly lower than an initial or other appropriate reference level, which may, for example, be a baseline level of activity and/or expression of the target in the absence or prior to administration of a composition described herein.

Inhibitor: As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.). In some embodiments, an inhibitor may be a small molecule, a polynucleotide, an oligonucleotide, a polysaccharide, a polypeptide, a protein, an antibody, and/or a functional portion thereof.

Inhibitor of a proinflammatory pathway: The term “inhibitor of a proinflammatory pathway” as used herein, in some embodiments, refers to an agent that inhibits or reduces inflammation that is associated with immunosuppression. In some embodiments, such an inhibitor of a proinflammatory pathway refers to an agent that prevents recruitment of immunosuppressive cells or prevents acute inflammation. Such acute inflammation and/or recruitment of immunosuppressive cells can occur after local trauma, including that which is caused by surgery. In some embodiments, an inhibitor of a proinflammatory pathway may inhibit, for example, an immune response that induces inflammation, including, e.g., production of inflammatory cytokines (including, e.g., but not limited to TGF-β and IL-10), increased activity and/or proliferation of M2-like macrophages, recruitment of relevant immune cells including, e.g., but not limited to myeloid cells, neutrophils, and mast cells, etc. Examples of inhibitors of a proinflammatory pathway include, e.g., ones described in International Application Number WO 2019/183216, the contents of which are incorporated herein by reference in their entirety for the purposes described herein.

Lymph node: As is known in the art, the term “lymph node” refers to components of the lymphatic system that are small structures, located throughout the body, through which lymph fluid flows. Lymph nodes are understood to filter certain substances from lymphatic fluid. Lymph nodes also can contain immune cells, for example that may participate in immune reactions throughout the body. In some embodiments, a lymph node may be or comprise a sentinel lymph node (i.e., a lymph node to which cancer cells are most likely to spread from a primary tumor).

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

Isomers: It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Metastasis: The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

Microparticle: As used herein, the term “microparticle” refers to a particle having a longest dimension (e.g., diameter) between 1 micrometer and 1,000 micrometers (μm). In some embodiments, a microparticle may be characterized by a longest dimension (e.g., a diameter) of between 1 μm and 500 μm. In some embodiments, a microparticle may be characterized by a longest dimension (e.g., a diameter) of between 1 μm and 100 μm. In many embodiments, a population of microparticles is characterized by an average size (e.g., longest dimension) that is below about 1,000 μm, about 500 μm, about 100 μm, about 50 μm, about 40 μm, about 30 μm, about 20 μm, or about 10 μm and often above about 1 μm. In many embodiments, a microparticle may be substantially spherical (e.g., so that its longest dimension may be its diameter.

Monosaccharide: As used herein, the term “monosaccharide” is given its ordinary meaning as used in the art and refers to a simple form of a sugar that consists of a single saccharide unit which cannot be further decomposed to smaller saccharide building blocks or moieties. Common examples of monosaccharides include, e.g., glucose (dextrose), fructose, galactose, mannose, ribose, etc. Monosaccharides can be classified according to the number of carbon atoms of the carbohydrate, for example, triose, having 3 carbon atoms such as glyceraldehyde and/or dihydroxyacetone; tetrose, having 4 carbon atoms such as erythrose, threose and/or erythrulose; pentose, having 5 carbon atoms such as arabinose, lyxose, ribose, xylose, ribulose and/or xylulose; hexose, having 6 carbon atoms such as allose, altrose, galactose, glucose, gulose, idose, mannose, talose, fructose, psicose, sorbose and/or tagatose; heptose, having 7 carbon atoms such as mannoheptulose, and/or sedoheptulose; octose, having 8 carbon atoms such as 2-keto-3-deoxy-manno-octonate; nonose, having 9 carbon atoms such as sialose; and decose, having 10 carbon atoms. The above monosaccharides encompass both D- and L-monosaccharides. Alternatively, a monosaccharide can be a monosaccharide variant, in which the saccharide unit comprises one or more substituents (e.g., deoxy, H substituents, heteroatom substituents (e.g., S, Cl, F, etc.), etc.) other than a hydroxyl. Such variants can be, but are not limited to, ethers, esters, amides, acids, phosphates and amines. Amine variants (i.e., amino sugars) include, for example, glucosamine, galactosamine, fructosamine and/or mannosamine. Amide variants include, for example, N-acetylated amine variants of saccharides (e.g., N-acetylglucosamine, and/or N-acetylgalactosamine).

Modulator: As used herein, the term “modulator” may be or comprise an entity whose presence or level in a system in which an activity of interest is observed correlates with a change in level and/or nature of that activity as compared with that observed under otherwise comparable conditions when the modulator is absent. In some embodiments, a modulator is an activator or agonist, in that an activity of interest is increased in its presence as compared with that observed under otherwise comparable conditions when the modulator is absent. In some embodiments, a modulator is an antagonist or inhibitor, in that an activity of interest is reduced in its presence as compared with otherwise comparable conditions when the modulator is absent. In some embodiments, a modulator interacts directly with a target entity whose activity is of interest. In some embodiments, a modulator interacts indirectly (e.g., interacts with one or more entities that interacts and/or are associated with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level. In some embodiments, a modulator may be a small molecule, a polynucleotide, an oligonucleotide, a polysaccharide, a polypeptide, a protein, an antibody, and/or a functional portion thereof.

Modulator of macrophage effector function: The term “modulator of macrophage effector function” refers to an agent that activates macrophage effector function or depletes immunosuppressive macrophages or macrophage-derived suppressor cells. Such potentiation can mobilize macrophage and myeloid components to destroy the tumor and its stroma, including the tumor vasculature. Macrophages can be induced to secrete antitumor cytokines and/or to perform phagocytosis, including antibody-dependent cellular phagocytosis.

Modulator of neutrophil function: As used interchangeably herein, the terms “modulator of neutrophils” and “modulator of neutrophil function” refer to a modulator of one or more biological functions and/or phenotypes of neutrophils. For example, in some embodiments, a modulator of neutrophil function can inhibit recruitment, survival, and/or proliferation of neutrophils. Additionally or alternatively, in some embodiments, a modulator of neutrophil function can modulate neutrophil-associated effector function, which may include but are not limited to, modulation of production and/or secretion of one or more immunomodulatory molecules (e.g., immunomodulatory cytokines and/or chemokines) and/or alter extracellular-matrix modifying capabilities of neutrophils. In some embodiments, a modulator of neutrophil function (e.g., ones described herein) may act on or target neutrophils only. In some embodiments, a modulator of neutrophil function (e.g., ones described herein) may act on neutrophils and at least one additional type of immune cells, e.g., other subsets of myeloid-derived suppressive cells (MDSCs), macrophages, and/or monocytes. One of ordinary skill in the art will appreciate that at least a subset of neutrophils may exhibit similar immune activities as one or more certain subsets of MDSCs and thus be considered as polymorphonuclear and/or granulocytic MDSCs (for example, as described in: Mehmeti-Ajradini et al., “Human G-MDSCs are neutrophils at distinct maturation stages promoting tumor growth in breast cancer” Life Science Alliance, Sep. 21, 2020; and Brandau et al., “A subset of mature neutrophils contains the strongest PMN-MDSC activity in blood and tissue of patients with head and neck cancer” The Journal of Immunology, May 1, 2020; the contents of each of which are incorporated herein by reference for purposes described herein).

Nanoparticle: As used herein, the term “nanoparticle” refers to a particle having a longest dimension (e.g., a diameter) of less than 1000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 nm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

Neoplasm and tumor: The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

Nucleic acid: The term “nucleic acid” as used herein is in alignment with its ordinary meaning, and may also be represented as polynucleotide. However, when used as “a nucleic acid” or “at least one nucleic acid”, this does not refer to a single molecule, but to a single species of nucleic acid.

Payload: In general, the term “payload”, as used herein, refers to an agent that may be incorporated into a polymeric biomaterial(s) preparation described herein. In some embodiments, a payload may refer to a compound, molecule, or entity of any chemical class including, for example, a small molecule, a peptide, a polypeptide, a nucleic acid, a saccharide (e.g., a polysaccharide), a lipid, a metal, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, a mineral, a growth factor, a cytokine, an antibody, a hormone, an extracellular matrix protein (such as collagen, vitronectin, fibrin, etc.), an extracellular matrix sugar, a chemoattractant, a polynucleotide (e.g., DNA, RNA, antisense molecule, plasmid, etc.), a microorganism (e.g., a virus), etc., or a combination thereof. In some embodiments, a payload is or comprises a therapeutic agent. Examples of a therapeutic agent include but are not limited to analgesics, antibiotics, antibodies, anticoagulants, antiemetics, cells, coagulants, cytokines, growth factors, hormones, immunomodulatory agents, polynucleotides (e.g., DNA, RNA, antisense molecules, plasmids, etc.), and combinations thereof. In some embodiments, a payload may be or comprise a cell or organism, or a fraction, extract, or component thereof. Alternatively or additionally, in some embodiments, a payload may be or comprise a natural product in that it is found in and/or is obtained from nature. Alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such an agent may be in crude form.

Pharmaceutically acceptable salt: The term “pharmaceutically acceptable salt” refers to those salts which are, within the scope of sound medical judgment, suitable for use in contact with the tissues of, for example, humans and/or animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, the contents of which are incorporated herein by reference for the purposes described herein. Pharmaceutically acceptable salts that may be utilized in accordance with certain embodiments of the present disclosure may include, for example, those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C1-C4 alkyl)4-salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Polynucleotide carrier: The term “polynucleotide carrier”, “polynucleotide agent carrier”, or “nucleic acid carrier” refers to any agent which facilitates the endocytosis of nucleic acids by eukaryotic cells. These can include, but are not limited to, lipids, proteins, and small molecules.

Poloxamer: As used herein, the term “poloxamer” refers to a polymer preparation of or comprising one or more poloxamers. In some embodiments, poloxamers in a polymer preparation may be unconjugated or unmodified, for example, which are typically triblock copolymers comprising a hydrophobic chain of polyoxypropylene (polypropylene glycol, PPG) flanked by two hydrophilic chains of polyoxyethylene (polyethylene glycol, PEG). In some embodiments, a polymer preparation of or comprising one or more poloxamer may be unfiltered (e.g., such a polymer preparation may contain impurities and/or relatively low molecular weight polymeric molecules, as compared to a comparable polymer preparation that is filtered). Examples of poloxamers include are not limited to, Poloxamer 124 (P124, also known as Pluronic L44 NF), Poloxamer 188 (P188, also known as Pluronic F68NF), Poloxamer 237 (P237, also known as Pluronic F 87 NF), Poloxamer 338 (P338, also known as Pluronic F108 NF), Poloxamer 407 (P407, also known as Pluronic F127 NF), and combinations thereof.

Polymer: The term “polymer” is given its ordinary meaning as used in the art, i.e., a molecular structure comprising one or more repeat units (monomers), connected by covalent bonds. The repeat units may all be identical, or, in some cases, there may be more than one type of repeat unit present within the polymer (e.g., in a copolymer). In certain embodiments, a polymer is naturally occurring. In certain embodiments, a polymer is synthetic (i.e., not naturally occurring). In some embodiments, a polymer is a linear polymer. In some embodiments, a polymer is a branched polymer. In some embodiments, a polymer for use in accordance with the present disclosure is not a polypeptide. In some embodiments, a polymer for use in accordance with the present disclosure is not a nucleic acid. In some embodiments, a polymer for use in accordance with the present disclosure is not a lipid.

Polymer combination preparation: As used herein, the term “polymer combination preparation” refers to a polymeric biomaterial comprising at least two distinct polymer components. For example, in many embodiments, a polymer combination preparation described herein is a polymeric biomaterial comprising a first polymer component and a second first polymer component, wherein the first polymer component is or comprises at least one poloxamer, and the second polymer component is or comprises a polymer that is not poloxamer. In some embodiments, a polymer combination preparation described herein is a polymeric biomaterial in a precursor state, which may be, e.g., useful for administration to a subject. In some embodiments, a polymer combination preparation described herein is a polymeric biomaterial in a polymer network state.

Polymeric biomaterial: A “polymeric biomaterial”, as described herein, is a material that is or comprises at least one polymer or at least one polymeric moiety and is biocompatible. In many embodiments, a polymeric biomaterial is or includes at least one polymer; in some embodiments, a polymer may be or comprise a copolymer. In some embodiments, a polymeric biomaterial is or comprises a preparation of at least two distinct polymer components (e.g., a preparation containing poloxamer and a second polymer component that is not a poloxamer). Those skilled in the art will be aware that certain polymers may exist and/or be available in a variety of forms (e.g., length, molecular weight, charge, topography, surface chemistry, degree and/or type of modification such as alkylation, acylation, quaternization, hydroxyalkylation, carboxyalkylation, thiolation, phosphorylation, glycosylation, etc.); in some embodiments, a preparation of such polymers may include a specified level and/or distribution of such form or forms. Additionally or alternatively, those skilled in the art will appreciate that, in some embodiments, one or more immunomodulatory properties of a polymeric biomaterial may be tuned by its biomaterial property(ies), including, e.g., surface chemistry of a polymeric biomaterial (e.g., modulated by hydrophobicity and/or hydrophilicity portions of a polymeric biomaterial, chemical moieties, and/or charge characteristics) and/or topography of a polymeric biomaterial (e.g., modulated by size, shape, and/or surface texture), for example as described in Mariani et al. “Biomaterials: Foreign Bodies or Tuners for the Immune Response?” International Journal of Molecular Sciences, 2019, 20, 636; the contents of which are incorporated herein in their entirety by reference for the purposes described herein. In some embodiments, a polymeric biomaterial can be in a polymer network state. In some embodiments, a polymeric biomaterial can be in an injectable format, e.g., in a precursor state (e.g., a viscous solution). For example, a polymeric biomaterial can comprise its precursor components to be formed in situ (e.g., upon administration to a subject). In some embodiments, a polymeric biomaterial can be a liquid. In some embodiments, a polymeric biomaterial is a viscous solution. In some embodiments, a polymeric biomaterial is a colloid. In some embodiments, a polymeric biomaterial can be a solid. In some embodiments, a polymeric biomaterial can be a crystal (e.g., an inorganic crystal).

Polymer network: The term “polymer network” is used herein to describe an assembly of polymer chains interacting with each other. In some embodiments, a polymer network forms a three-dimensional structure material. In some embodiments, a polymer network may be formed by linking polymer chains (“crosslinked polymer network”) using a crosslinker (e.g., as described herein). In some embodiments, a polymer network is transitioned from a precursor state when it is exposed to a temperature that is or above a critical gelation temperature, wherein the polymer network state has a viscosity materially above (e.g., at least 50% or above) that of the precursor state and the polymer network state comprises crosslinks not present in the precursor state. In some embodiments, a polymer network may be formed by non-covalent or non-ionic intermolecular association of polymer chains, e.g., through hydrogen bonding. In some embodiments, a polymer network may be formed by a combination of chemically crosslinking polymer chains and non-covalent or non-ionic intermolecular association of polymer chains.

Proinflammatory cytokine: As used herein, the term “proinflammatory cytokine” refers to a protein or glycoprotein molecule secreted by a cell (e.g., a cell of an immune system) that induces an inflammatory response. As will be appreciated by one of skilled in the art, inflammation may be immunostimulatory or immunosuppressive depending on the biological context.

Proinflammatory immune response: The term “proinflammatory immune response” as used herein refers to an immune response that induces inflammation, including, e.g., production of proinflammatory cytokines (including, e.g., but not limited to CXCL10, IFN-α, IFN-β, IL-1β, IL-6, IL-18, and/or TNF-alpha), increased activity and/or proliferation of Th1 cells, recruitment of myeloid cells, etc. In some embodiments, a proinflammatory immune response may be or comprise one or both of acute inflammation and chronic inflammation.

Proliferative disease: A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

Prophylactically 1 effective amount: A “prophylactically effective amount” is an amount sufficient to prevent (e.g., significantly delay onset or recurrence of one or more symptoms or characteristics of, for example so that it/they is/are not detected at a time point at which they would be expected absent administration of the amount) a condition. A prophylactically effective amount of a composition means an amount of therapeutic agent(s), alone or in combination with other agents, that provides a prophylactic benefit in the prevention of the condition. The term “prophylactically effective amount” can encompass an amount that improves overall prophylaxis or enhances the prophylactic efficacy of another prophylactic agent. Those skilled in the art will appreciate that a prophylactically effective amount need not be contained in a single dosage form. Rather, administration of an effective amount may involve administration of a plurality of doses, potentially over time (e.g., according to a dosing regimen).

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0,1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

Salt: As used herein, the term “salt” refers to any and all salts and encompasses pharmaceutically acceptable salts.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Small molecule: The term “small molecule” or “small molecule therapeutic” refers to a molecule, whether naturally occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, a small molecule is a therapeutically active agent such as a drug (e.g., a molecule approved by the U.S. Food and Drug Administration as provided in the Code of Federal Regulations (C.F.R.)). A small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, a small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, the contents of each of which are incorporated herein by reference for purposes described herein; such listed drugs are typically considered acceptable for use in accordance with the present disclosure.

Solvate: The term “solvate”, as used herein, has its art-understood meaning and refers to an aggregate of a compound (which may, for example, be a salt form of the compound) and one or more solvent atoms or molecules. In some embodiments, a solvate is a liquid. In some embodiments, a solvate is a solid form (e.g., a crystalline form). In some embodiments, a solid-form solvate is amenable to isolation. In some embodiments, association between solvent atom(s) and compound in a solvate is a non-covalent association. In some embodiments, such association is or comprises hydrogen bonding, van der Waals interactions, or combinations thereof. In some embodiments, a solvent whose atom(s) is/are included in a solvate may be or comprise one or more of water, methanol, ethanol, acetic acid, DMSO, THF, diethyl ether, and the like. Suitable solvates may be pharmaceutically acceptable solvates; in some particular embodiments, solvates are hydrates, ethanolates, or methanolates. In some embodiments, a solvate may be a stoichiometric solvate or a non-stoichiometric solvate.

Subject: A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a tumor resection subject, e.g., a subject who has recently undergone tumor resection. In some embodiments, a tumor resection subject is a subject who has undergone tumor resection in less than 72 hours (including, e.g., less than 48 hours, less than 24 hours, less than 12 hours, less than 6 hours, or lower), prior to receiving a composition described herein. In some embodiments, a tumor resection subject is a subject who has undergone tumor resection in less than 48 hours, prior to receiving a composition described herein. In some embodiments, a tumor resection subject is a subject who has undergone tumor resection in less than 24 hours prior to receiving a composition described herein. In some embodiments, a tumor resection subject is a subject who has undergone tumor resection in less than 12 hours, prior to receiving a composition described herein.

Substantially: As used herein, the term “substantially” refers to the qualitative condition of exhibiting total or near-total extent or degree of a characteristic or property of interest. Those skilled in the art will understand that an agent of interest, if ever, achieves or avoids an absolute result, e.g., an agent of interest that indeed has zero effect on an immune response, e.g., inflammation. The term “substantially” is therefore used herein to capture the potential lack of absoluteness inherent in many biological and chemical effects.

Sustained: As used interchangeably herein, the term “sustained” or “extended” typically refers to prolonging an effect and/or a process over a desirable period of time. For example, in the context of sustained immunomodulation (e.g., in the presence of a composition or preparation as described herein and/or utilized herein), such an immunomodulatory effect may be observed for a longer period of time after administration of a particular immunomodulatory payload in the context of a composition comprising a biomaterial preparation and otherwise as described herein, as compared to that which is observed with administration of the same payload absent such a biomaterial preparation. In the context of sustained release of one or more agents of interest (e.g., one or more polynucleotide agents that encode or regulate an immunomodulatory polypeptide incorporated in biomaterial preparations described herein) from compositions described herein over a period of time, such release may occur on a timescale within a range of from about 30 minutes to several weeks or more. In some embodiments, the extent of sustained release or extended release can be characterized in vitro or in vivo. For example, in some embodiments, release kinetics can be tested in vitro by placing a preparation and/or composition described herein in an aqueous buffered solution (e.g., PBS at pH 7.4). In some embodiments, when a composition described herein is placed in an aqueous buffered solution (e.g., PBS at pH 7.4), less than 100% or lower (including, e.g., less than or equal to 90%, less than or equal to 80%, less than or equal to 70%, less than or equal to 50% or lower) of one or more agents of interest (e.g., one or more polynucleotide agents that encode or regulate an immunomodulatory polypeptide incorporated in biomaterial preparations described herein) is released within 3 hours from a biomaterial. In some embodiments, release kinetics can be tested in vivo, for example, by administering (e.g., implanting) a composition at a target site (e.g., mammary fat pad) of an animal subject (e.g., a mouse subject). In some embodiments, when a composition is administered (e.g., implanted) at a target site (e.g., mammary fat pad) of an animal subject (e.g., a mouse subject), less than or equal to 70% or lower (including, e.g., less than or equal to 60%, less than or equal to 50%, less than 40%, less than 30% or lower) of one or more agents of interest (e.g., one or more polynucleotide agents that encode or regulate an immunomodulatory polypeptide incorporated in biomaterial preparations described herein) is released in vivo 8 hours after the

Targeted agent: The term “targeted agent”, when used in reference to an anticancer agent means one that blocks the growth and spread of cancer by interfering with specific molecules (“molecular targets”) that are involved in the growth, progression, and/or spread of cancer. Targeted agents are sometimes called “targeted cancer therapies,” “molecularly targeted drugs,” “molecularly targeted therapies,” or “precision medicines.” Targeted agents differ from traditional chemotherapy in that targeted agents typically act on specific molecular targets that are specifically associated with cancer, and/or with a particular tumor or tumor type, stage, etc., whereas many chemotherapeutic agents act on all rapidly dividing cells (e.g., whether or not the cells are cancerous). Targeted agents are deliberately chosen or designed to interact with their target, whereas many standard chemotherapies are identified because they kill cells.

Tautomers: The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

Test subject: As used herein, the term “test subject” refers to a subject to which technologies provided herein are applied for experimental investigation, e.g., to assess biomaterial degradation, and/or efficacy of compositions and/or preparations described herein in antitumor immunity. In some embodiments, a test subject may be a human subject or a population of human subjects. For example, in some embodiments, a human test subject may be a normal healthy subject. In some embodiments, a human test subject may be a tumor resection subject. In some embodiments, a test subject may be a mammalian non-human animal or a population of mammalian non-human animals. Non-limiting examples of such mammalian non-human animals include mice, rats, dogs, pigs, rabbits, etc., which in some embodiments may be normal healthy subjects, while in some embodiments may be tumor resection subjects. In some embodiments, mammalian non-human animals may be transgenic or genetically engineered animals.

Therapeutic agent: The term “therapeutic agent” refers to an agent having one or more properties that produce a desired, usually beneficial, physiological effect. For example, a therapeutic agent may treat, ameliorate, and/or prevent disease. Those skilled in the art, reading the present disclosure, will appreciate that the term “therapeutic agent”, as used herein, does not require a particular level or type of therapeutic activity, such as might be required for a regulatory agency to consider an agent to be “therapeutically active” for regulatory purposes. As will be understood by those skill in the art, reading the present disclosure, in some embodiments, certain polymeric biomaterial(s) preparations described herein (in the absence of an immunomodulatory payload) may have one or more properties that contribute to and/or achieve a desired physiological effect, and therefore may be considered to be a “therapeutic agent” as that term is used here (whether or not such biomaterial would or would not be considered to be pharmaceutically active by any particular regulatory agency). In some embodiments, a therapeutic agent that may be utilized in preparations, compositions and/or methods described herein (e.g., involving polymeric biomaterial(s) preparations described herein) may be or comprise an immunomodulatory payload. In some embodiments, a therapeutic agent that may be utilized in preparations, compositions and/or methods described herein (e.g., involving polymeric biomaterial(s) preparations described herein) may be or comprise a non-immunomodulatory payload, e.g., comprising a biologic, a small molecule, nucleic acid, polypeptide, or a combination thereof. In some embodiments, a therapeutic agent that may be utilized in preparations, compositions and/or methods described herein (e.g., involving polymeric biomaterial(s) preparations described herein) may be or comprise a chemotherapeutic agent, which in some embodiments may be or comprise a cytotoxic agent.

Therapeutically effective amount: A “therapeutically effective amount” is an amount sufficient to provide a therapeutic benefit in the treatment of a condition, which therapeutic benefit may be or comprise, for example, reduction in frequency and/or severity, and/or delay of onset of one or more features or symptoms associated with the condition. A therapeutically effective amount means an amount of therapeutic agent(s), alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. Those skilled in the art will appreciate that a therapeutically effective amount need not be contained in a single dosage form. Rather, administration of an effective amount may involve administration of a plurality of doses, potentially over time (e.g., according to a dosing regimen, and particularly according to a dosing regimen that has been established, when applied to a relevant population, to provide an appropriate effect with a desired degree of statistical confidence).

Temperature-responsive: As used herein, the term “temperature-responsive”, in the context of a temperature-responsive polymer or biomaterial (e.g., a polymeric biomaterial), refers to a polymer or biomaterial (e.g., polymeric biomaterial) that exhibits an instantaneous or discontinuous change in one or more of its properties at a critical temperature (e.g., a critical gelation temperature). For example, in some embodiments, one or more of such properties is or comprise a polymer's or biomaterial's solubility in a particular solvent. By way of example only, in some embodiments, a temperature-responsive polymer or biomaterial (e.g., polymeric biomaterial) is characterized in that it is a homogenous polymer solution or colloid that is stable below a critical temperature (e.g., a critical gelation temperature) and instantaneously form a polymer network (e.g., a hydrogel) when the critical temperature (e.g., critical gelation temperature) has been reached or exceeded. In some embodiments, a temperature-responsive polymer or biomaterial (e.g., polymeric biomaterial) may be temperature-reversible, e.g., in some embodiments where a polymer solution may instantaneously form a polymer network at a temperature of or above a critical gelation temperature, and such a resulting polymer network may instantaneously revert to a homogenous polymer solution when the temperature is reduced to below the critical gelation temperature.

Treatment: The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein, e.g., cancer or tumor. In some embodiments, treatment may be administered after one or more signs or symptoms have developed or have been observed. Treatment may also be continued after symptoms have resolved, for example, to delay or prevent recurrence and/or spread.

Tumor: The terms “tumor” and “neoplasm” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

Tumor removal: As used herein, the term “tumor removal” encompasses partial or complete removal of a tumor, which may be resulted from a cancer therapy, e.g., surgical resection. In some embodiments, tumor removal refers to physical removal of part or all of a tumor by surgery (i.e., “tumor resection”). In some embodiments, tumor removal may be resulted from a surgical tumor resection and an adjuvant therapy (e.g., chemotherapy, immunotherapy, and/or radiation therapy). In some embodiments, an adjuvant therapy may be administered after a surgical tumor resection, e.g., at least 24 hours or more after a surgical tumor resection.

Tumor resection subject: As used herein, the term “tumor resection subject” refers to a subject who is undergoing or has recently undergone a tumor resection procedure. In some embodiments, a tumor resection subject is a subject who has at least 70% or more (including, at least 80%, at least 90%, at least 95%, at least 98%, at least 99%, or higher (including 100%) of gross tumor mass removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection subject may be determined to have a negative resection margin (i.e., no cancer cells seen microscopically at the resection margin, e.g., based on histological assessment of tissues surrounding the tumor resection site). In some embodiments, a tumor resection subject may be determined to have a positive resection margin (i.e., cancer cells are seen microscopically at the resection margin, e.g., based on histological assessment of tissues surrounding the tumor resection site). In some embodiments, a tumor resection subject may have micrometastases and/or dormant disseminated cancer cells that can be driven to progress/proliferate by the physiologic response to surgery. In some embodiments, a tumor resection subject receives a composition (e.g., as described and/or utilized herein) immediately after the tumor resection procedure is performed (e.g., intraoperative administration). In some embodiments, a tumor resection subject receives a composition (e.g., as described and/or utilized herein) postoperatively within 24 hours or less, including, e.g., within 18 hours, within 12 hours, within 6 hours, within 3 hours, within 2 hours, within 1 hour, within 30 minutes, or less.

Tumor resection site: The term “tumor resection site” generally means a site in which part or all of a tumor was or is being removed through tumor resection. In some embodiments, the term “tumor resection site” refers to a site in which at least 70% or more (including, at least 80%, at least 90%, at least 95%, at least 98%, at least 99%, or higher (including 100%) of gross tumor mass is removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection site may be determined to have a negative resection margin (i.e., no cancer cells seen microscopically at the resection margin, e.g., based on histological assessment of tissues surrounding the tumor resection site). In some embodiments, a tumor resection site may be determined to have a positive resection margin (i.e., cancer cells are seen microscopically at the resection margin, e.g., based on histological assessment of tissues surrounding the tumor resection site).

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant biomaterial (e.g., a variant polymer or a polymeric biomaterial comprising a variant polymer) may differ from a reference biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can retain the desired property(ies) and/or function(s) (e.g., immunomodulation and/or temperature-responsiveness) of the reference biomaterial. For example, a variant of an immunomodulatory biomaterial may differ from a reference immunomodulatory biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can act on an immune system (e.g., by stimulating innate immunity), e.g., when used in a method described herein. In some embodiments, a variant immunomodulatory biomaterial (e.g., a variant polymer or a polymeric biomaterial comprising a variant polymer) is characterized in that, when assessed at 24 hours after administration of such a variant immunomodulatory biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 60% or more (e.g., including, e.g., at least 70%, at least 80%, at least 90%, at least 95%, at least 98%, or up to 100%) of that observed when a reference biomaterial (e.g., a reference polymer or polymeric biomaterial) is administered at the target site. In some embodiments, a variant immunomodulatory biomaterial (e.g., a variant polymer or a polymeric biomaterial comprising a variant polymer) is characterized in that, when assessed at 24 hours after administration of such a variant biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 1.1-fold or more (e.g., including, e.g., at least 1.5-fold, at least 2-fold, at least 3-fold, at least 4-fold, at least 5-fold, or more) of that observed when a reference biomaterial (e.g., a reference polymeric biomaterial) is administered at the target site.

In some embodiments, a variant biomaterial (e.g., a variant polymeric biomaterial) exhibits at least one physical characteristic that is different from that of a reference biomaterial (e.g., a reference polymeric biomaterial). For example, in some embodiments, a variant biomaterial (e.g., a variant polymeric biomaterial) can exhibit increased water solubility (e.g., at a physiological pH) as compared to that of a reference biomaterial (e.g., a reference polymeric biomaterial). In some embodiments, a variant has 10, 9, 8, 7, 6, 5, 4, 3, 2, or 1 structural modifications as compared with a reference. In some embodiments, a variant has a small number (e.g., fewer than 5, 4, 3, 2, or 1) number of structural modifications (e.g., alkylation, acylation, quaternization, hydroxyalkylation, carboxyalkylation, thiolation, phosphorylation, glycosylation, etc.). In some embodiments, a variant has not more than 5, 4, 3, 2, or 1 additions or deletions of chemical moieties, and in some embodiments has no additions or deletions, as compared with a reference. In some embodiments, a variant is an entity that can be generated from a reference by chemical manipulation. In some embodiments, a variant is an entity that can be generated through performance of a synthetic process substantially similar to (e.g., sharing a plurality of steps with) one that generates a reference.

### DETAILED DESCRIPTION OF CERTAIN EMBODIMENTS

Provided herein are compositions comprising at least one or more polymeric biomaterials and one or more nucleic acids. In some embodiments, provided compositions can localize delivery of one or more nucleic acids, which may encode or directly function as active agents (e.g., immunomodulatory agents), to a target site (e.g., a site at which a tumor has been removed and/or cancer cells have been treated or killed, e.g., by chemotherapy or radiation and/or in/near a tumor which has not been treated) and thereby concentrate the action of the active agents to a target site in need thereof. Such compositions can be particularly useful for treating cancer.

In some embodiments, compositions may comprise polymeric biomaterial(s), nucleic acid(s), and optionally one or more additional immunomodulatory molecules. In some embodiments, provided compositions may comprise polymeric biomaterial(s), and nucleic acid(s) which function as modulators of the adaptive immune response and/or modulators of the innate immune response. In some embodiments, the additional immunomodulatory molecule may comprise inhibitor(s) of the proinflammatory immune response mediated by p38 mitogen-activated protein kinase (MAPK) pathway (e.g., a p38 MAPK inhibitor). In some embodiments, compositions may comprise an activator of the innate immune response, cytokines, and/or chemokines. In some embodiments, compositions may comprise one or more activators of adaptive immune response. In some embodiments, provided compositions may further comprise additional therapeutic agents (e.g., additional inhibitor of a pro-inflammatory pathway, modulators of macrophage effector function, and/or chemotherapeutic agents etc.).

In some embodiments, compositions may comprise therapeutic agents (e.g., nucleic acids which act as or encode immunomodulatory agents) that may mediate or inhibit inflammation (e.g., chronic inflammation) induced by trauma or microtrauma (e.g., by surgery such as surgical tumor resection, keyhole surgery, or injection), thus providing unique tools for the treatment of cancer, particularly solid tumors. In some embodiments, provided therapeutic agents may modify and/or reduce or inhibit activity of myeloid-derived suppressor cells (MDSCs). In some embodiments, provided therapeutic agents may modify and/or reduce or inhibit activity of Neutrophils. In some embodiments, provided therapeutic agents may modify and/or reduce or inhibit activity of Macrophages. In some embodiments, provided therapeutic agents may modify and/or reduce or inhibit recruitment of immunosuppressive cells. In some embodiments, provided therapeutic agents may modify and/or reduce or inhibit acute inflammation. In some embodiments, provided therapeutic agents may activate the innate immune response system and/or the adaptive immune response system. Compositions, methods, and kits provided herein are also advantageous over existing methods in that they do not require administration of cells (e.g., adoptive cell transfer) or incorporation or presence of additional components such as nanoparticles, microparticles, certain peptides, or tumor antigens.

In some embodiments, compositions described herein are useful for treating cancer (e.g., solid tumors or metastases) in the perioperative setting. In some embodiments, compositions may deliver immunotherapies by implantation or injection of the compositions at the site of therapeutic need in a subject in need thereof. In some embodiments, compositions provided herein are particularly advantageous over existing immunotherapies at least because, in some embodiments, they can release an immunomodulatory agent (e.g., a nucleic acid acting as or encoding an immunomodulatory agent) directly to a site of tumor resection, avoiding systemic administration. Accordingly, some embodiments of compositions described herein provide a vehicle for drug delivery at the site of tumor resection that avoids potential toxicities that can be associated with traditional systemic administration of immunotherapies. Concentrating the immunotherapy at the site of tumor resection can similarly improve efficacy. In certain embodiments, provided compositions are useful for slowing and/or impeding tumor growth, preventing cancer recurrence, preventing tumor metastasis, and/or preventing primary tumor regrowth.

Among other things, in some embodiments, the present disclosure provides technologies for suppression of immune responses that may themselves foster additional immunosuppression (e.g., activity of certain Macrophages, Neutrophils, and/or MDSCs).

Without wishing to be bound by any particular theory, the present disclosure notes that, in some embodiments, technologies provided herein may reduce a type of inflammation that is generally observed in the context of chronic inflammation (e.g., as is often associated with an autoimmune disease) but, as described herein, may be activated in an acute setting (i.e., post-surgery). The present disclosure provides the insight that therapy targeting inflammation by harnessing the power of localized delivery of nucleic acids as described herein may be uniquely useful in the post-tumor resection context. Those skilled in the art will appreciate that many therapeutic strategies designed and/or effective to ameliorate or stimulate autoimmune responses could be disastrous in the setting of tumor resection, as they could result in worsening of the tumor progression phenotype. The present disclosure teaches that, notwithstanding this general principle, targeting certain immunomodulatory pathways through targeted and/or localized nucleic acid administration as described herein is surprisingly useful in the cancer therapy context.

In some embodiments, described compositions comprising polymeric biomaterials and nucleic acids may be combined, for example, with therapies that include other immune modulation strategies such as, for example, activation/agonism of innate immune system through small molecule or peptide administration (e.g., via administration of an agent such as a STING agonist or a TLR agonist).

### Compositions

### Polymeric Biomaterials

Compositions described herein include at least one biomaterial, generally referred to throughout as polymeric biomaterial(s). In some embodiments, the polymeric biomaterial can form a polymer network which can act as a scaffold or depot for dispersal of at least some of the additional components comprised within the composition (e.g., nucleic acids). In some embodiments, the scaffold or depot comprises any synthetic or naturally occurring material that is suitable for containing and promoting the sustained or extended release of any therapeutic agents in compositions as described herein. Accordingly, a polymeric biomaterial possess the potential to have physical properties that provide some of the advantageous properties of the compositions described herein (e.g., storage modulus, biodegradation, and/or release profile of therapeutic agents).

In certain embodiments, compositions comprise polymeric biomaterial(s) that can extend the release of a therapeutic agent (e.g., nucleic acid) when delivered to a target site (e.g., tumor resection site) relative to administration of the same therapeutic agent in solution. In certain embodiments, polymeric biomaterial(s) extend the release of a therapeutic agent in the tumor resection site relative to administration of the same therapeutic agent in solution by at least 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, 2 weeks, 3 weeks, or 4 weeks. In some embodiments, polymeric biomaterial(s) extend release of at least one therapeutic agent (e.g., a nucleic acid encoding or acting as an immunomodulatory agent) so that, when assessed at a specified time point after administration, more therapeutic agent is present in the tumor resection site relative to the levels observed when the therapeutic agent is administered in solution. For example, in some embodiments, when assessed at 24 hours after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution. In some embodiments, when assessed at 48 hours after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution. In some embodiments, when assessed at 3 days after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution. In some embodiments, when assessed at 5 days after administration, the amount of therapeutic agent released to and present in the tumor resection site is at least 30% more (including, e.g., at least 40%, at least 50%, at least 60%, at least 70%, at least 80%, at least 90%, or more) than that is observed when the therapeutic agent is administered in solution.

In some embodiments, compositions comprising a polymeric biomaterial(s) preparation described herein (e.g., a precursor state or a polymer network state) can be characterized by a viscosity of no more than 10,000 mPa·s or lower, including, e.g., no more than 9000 mPa·s, no more than 8000 mPa·s, no more than 7000 mPa·s, no more than 6000 mPa·s, no more than 5000 mPa·s, no more than 4000 mPa·s, no more than 3500 mPa·s, no more than 3000 mPa·s, no more than 2500 mPa·s, no more than 2000 mPa·s, no more than 1500 mPa·s, no more than 1000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, compositions comprising a polymeric biomaterial(s) preparation described herein (e.g., a precursor state or a polymer network state) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1000 mPa·s, at least 1500 mPa·s, at least 2000 mPa·s, at least 2500 mPa·s, at least 3000 mPa·s, at least 4000 mPa·s, at least 5000 mPa·s, at least 6000 mPa·s, at least 7000 mPa·s, at least 8000 mPa·s, at least 9000 mPa·s, or higher. Combinations of the above-mentioned ranges are also possible. For example, in some embodiments, compositions comprising a polymeric biomaterial(s) preparation described herein (e.g., a precursor state or a polymer network state) may be characterized by a viscosity of 5 mPa·s to 10,000 mPa·s, or 10 mPa·s to 5000 mPa·s, or 5 mPa·s to 200 mPa·s, or 20 mPa·s to 100 mPa·s, or 5 mPa·s to 20 mPa·s. One skilled in the art reading the present disclosure will appreciate that, in some cases, viscosity of a composition comprising a polymeric biomaterial(s) preparation described herein may be selected or adjusted based on, e.g., administration routes (e.g., injection vs. implantation), injection volume and/or time, and/or impact duration of innate immunity stimulation. As will be also understood by one skilled in the art, viscosity of a polymer depends on, e.g., temperature and concentration of the polymer in a testing sample. In some embodiments, viscosity of compositions comprising a polymeric biomaterial(s) preparation described herein may be measured at 20° C., e.g., with a shear rate of 1000 s⁻¹.

In some embodiments when compositions comprising a polymeric biomaterial(s) preparation described herein is in a polymer network state, such a polymer network state may be characterized by a storage modulus of at least 100 Pa, at least 200 Pa, at least 300 Pa, at least 400 Pa, at least 500 Pa, at least 600 Pa, at least 700 Pa, at least 800 Pa, at least 900 Pa, at least 1000 Pa, at least 1100 Pa, at least 1200 Pa, at least 1300 Pa, at least 1400 Pa, at least 1500 Pa, at least 1600 Pa, at least 1700 Pa, at least 1800 Pa, at least 1900 Pa, at least 2000 Pa, at least 2100 Pa, at least 2200 Pa, at least 2300 Pa, at least 2400 Pa, at least 2500 Pa, at least 2600 Pa, at least 2700 Pa, at least 2800 Pa, at least 2900 Pa, at least 3000 Pa, at least 3500 Pa, at least 4000 Pa, at least 4500 Pa, at least 5000 Pa, at least 6000 Pa, at least 7000 Pa, at least 8000 Pa, at least 9000 Pa, or higher. In some embodiments, such a polymer network state of compositions comprising a polymeric biomaterial(s) preparation described herein may be characterized by a storage modulus of no more than 10 kPa, no more than 9 kPa, no more than 8 kPa, no more than 7 kPa, no more than 6 kPa, or lower. Combinations of the above-mentioned ranges are also possible. For example, in some embodiments, such a polymer network state of compositions comprising a polymeric biomaterial(s) preparation described herein may be characterized by a storage modulus of 100 Pa to 10 kPa, or 200 Pa to 5000 Pa, or 300 Pa to 2500 Pa, or 500 Pa to 2500 Pa or 100 Pa to 500 Pa. One of those skilled in the art will appreciate that various rheological characterization methods (e.g., as described in Weng et al., “Rheological Characterization of in situ Crosslinkable Hydrogels Formulated from Oxidized Dextran and N-Carboxyethyl Chitosan” Biomacromolecules, 8:1109-1115 (2007)) can be used to measure storage modulus of a material, and that, in some cases, storage modulus of a material may be measured with a rheometer and/or dynamic mechanical analysis (DMA). One of those skilled in the art will also appreciate that rheological characterization can vary with surrounding condition, e.g., temperature and/or pH. Accordingly, in some embodiments, a provided polymer combination preparation is characterized by a storage modulus (e.g., as described herein) measured at a body temperature of a subject (e.g., 37° C. of a human subject), e.g., at a pH 5-8 or at a physiological pH (e.g., pH 7). As will be clear to one skilled in the art reading the disclosure provided herein, a storage modulus of a provided polymer combination preparation, e.g., in a form of particles, refers to a bulk storage modulus of particles in a population.

In certain embodiments, compositions comprising polymeric biomaterials are comprised of positively-charged polymers. In certain embodiments, polymeric biomaterials are comprised of negatively-charged polymer(s). In certain embodiments, polymeric biomaterials are comprised of neutral polymer(s). In certain embodiments, the polymeric biomaterial comprises hyaluronic acid, alginate, chitosan, chitin, chondroitin sulfate, dextran, gelatin, collagen, starch, cellulose, polysaccharide, fibrin, poly-L-Lysine, methylcellulose, ethylene-vinyl acetate (EVA), poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyphosphazene, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, and/or combinations and/or derivatives thereof.

In certain embodiments, compositions comprising at least one polymeric biomaterial are comprised of hydrophobic polymeric biomaterial(s). In certain embodiments, the hydrophobic polymeric biomaterial comprises ethylene-vinyl acetate (EVA), poly(lactic-co-glycolic) acid (PLGA), polylactic acid (PLA), polyglycolic acid (PGA), polydioxanone (PDO), polyhydroxybutyrate (PHB), polycaprolactone (PCL), poly(ester amide), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyphosphazene, polyanhydride, poly(N-vinyl-2-pyrrolidone) (PVP), poly(acrylic acid) (PAA), poly(methacrylic acid) (PMA), poly(alpha ester), poly(ortho ester), polyphosphoester, polyurethane, polycarbonate, polyamide, and/or polyhydroxyalkanoate. In some embodiments, use of a hydrophobic polymeric biomaterials may be particularly useful when the therapeutic agent(s) in a composition is hydrophilic. In some embodiments, wherein the polymeric biomaterial is hydrophobic, hydrophobic therapeutic agents would be expected to be released over longer periods of time (e.g., days/weeks) rather than a relatively shorter period of time (hours/days). Accordingly, in certain embodiments, when the polymeric biomaterial is a hydrophobic polymer, then the therapeutic agent(s) of the composition are hydrophilic molecules. Additionally, in certain embodiments, when the biomaterial is a hydrophobic polymer, then the therapeutic agent(s) of the composition are hydrophobic and/or neutral molecules.

In certain embodiments, provided compositions comprising at least one polymeric biomaterial comprise a cross-linked biologic. In certain embodiments, the biologic is cross-linked by the self-immolating cross-linker dithio-bis(ethyl 1 H-imidazole-1-carboxylate) (DIC). In certain embodiments, the resultant polymer network or hydrogel is loaded with a small molecule. In certain embodiments, the small molecule comprises a nucleic acid. In certain embodiments, provided compositions may comprise a polynucleotide and a polynucleotide agent carrier.

Polymeric biomaterials useful for compositions described herein are biocompatible. In some embodiments, polymeric biomaterials are biodegradable in vivo. In some embodiments, compositions provided herein are able to be degraded, chemically and/or biologically, within a physiological environment, such as within the body. Degradation of the provided compositions may occur at varying rates, depending on the components and polymeric biomaterials used. For example, in some embodiments, the half-life of the provided compositions (the time at which 50% of the composition is degraded into monomers and/or other non-polymeric moieties) may be on the order of days, weeks, months, or years. In some embodiments, compositions may be biologically degraded, e.g., by enzymatic activity or cellular machinery, in some cases, for example, through exposure to a lysozyme (e.g., having relatively low pH), or by simple hydrolysis. In some embodiments, provided compositions may be broken down into monomers and/or other non-polymeric moieties that cells can either reuse or dispose of without significant toxic effect on the cells. In some embodiments, provided compositions are stable in vivo such that they deliver drug to the intended target in a suitable amount of time.

In certain embodiments, compositions comprise polymeric biomaterial(s) which may form polymer networks with or without the addition of a cross-linking agent. In some embodiments, polymeric biomaterial(s) may form a polymer network biomaterial in less than 10 minutes. Embodiments of compositions described herein can be prepared by any method known in the art of pharmacology. In certain embodiments, such preparatory methods include the steps of adding a thiol-modified hyaluronic acid into a mold; adding a nucleic acid (e.g., a nucleic acid encoding or acting as a modulator of the immune response); optionally adding an activator of adaptive immune response to the mold; optionally adding a chemokine or cytokine to the mold; optionally adding an activator of innate immune response to the mold; adding a cross-linking agent to the mold (e.g., a thiol-reactive PEGDA cross-linker); and allowing the mixture to stand for at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 90 minutes, at least 2 hours, at least 3 hours, at least 4 hours, at least 5 hours, or at least 6 hours for solidification.

In certain embodiments, compositions comprise at least one biomaterial, wherein the biomaterial(s) can form a polymer network. In certain embodiments, the polymer network is a hydrogel. In certain embodiments, the polymer network is crosslinked. Polymer networks or hydrogels can be crosslinked using any methods known in the art, e.g., chemical crosslinking methods (e.g., by using a small-molecule cross-linker, which can be derived from a natural source or synthesized), polyelectrolyte crosslinking (e.g., mixing a polymer with a second polymer comprising an opposite charge), thermal-induced crosslinking, photo-induced crosslinking (e.g., using vinyl sulfone, methacrylate, acrylic acid), pH-induced crosslinking, and enzyme-catalyzed crosslinking. In some embodiments, one or more cross-linking methods described in Parhi, Adv Pharm Bull., Review 7 (4): 515-530 (2017); which is incorporated herein by reference for the purposes described herein, can be used in forming a polymer network or hydrogel. In some embodiments, a polymer network or hydrogel is a non-crosslinked polymer network or hydrogel.

In certain embodiments, compositions comprising polymeric biomaterials (e.g., crosslinked or non-crosslinked) comprises hyaluronic acid, alginate, chitosan, chondroitin sulfate, dextran, gelatin, collagen, starch, cellulose, polysaccharide, fibrin, poly-L-Lysine, methylcellulose, polyethylene glycol (PEG), PEG diacrylate (PEGDA), disulfide-containing PEGDA (PEGSSDA), PEG dimethacrylate (PEGDMA), poly(2-hydroxyethyl methacrylate) (pHEMA), poly(beta-amino ester) (PBAE), poly(aspartic acid), poly(glutamic acid), poly(propylene glycol) (PPG), poly(vinyl alcohol) (PVA), polyacetal, polyglycerol, polyglucuronic acid, and/or combinations thereof. In certain embodiments, when the polymeric biomaterial is a polymer network or hydrogel, then the therapeutic agent(s) of the composition are hydrophilic molecules. In certain embodiments, when the polymeric biomaterial is a polymer network or hydrogel, then the therapeutic agent(s) of the composition are hydrophobic molecules. In certain embodiments, when the polymeric biomaterial is a polymer network or hydrogel, then the therapeutic agent(s) of the composition are hydrophobic or hydrophilic molecules. In certain embodiments, when the polymeric biomaterial is a polymer network or hydrogel, then the therapeutic agent(s) of the composition are amphiphilic molecules (e.g., both hydrophobic and hydrophilic molecules).

In certain embodiments, compositions comprising a polymeric biomaterial are comprised of hyaluronic acid, chitosan, and/or alginate. In certain embodiments, the polymeric biomaterial is cross-linked hyaluronic acid, cross-linked chitosan and/or cross-linked alginate. In certain embodiments, the polymeric biomaterial comprises cross-linked hyaluronic acid, cross-linked chitosan, or cross-linked alginate. In certain embodiments, the polymer network or hydrogel is hyaluronic acid, chitosan, or alginate. In certain embodiments, the polymeric biomaterial comprises hyaluronic acid. In certain embodiments, the polymeric biomaterial comprises cross-linked hyaluronic acid. In certain embodiments, the polymeric biomaterial is hyaluronic acid. In certain embodiments, the polymeric biomaterial is cross-linked hyaluronic acid. In certain embodiments, the polymer network is composed of two or more different polymeric biomaterials. In certain embodiments, the polymer network or hydrogel comprises hyaluronic acid and a poloxamer.

### Exemplary Poloxamers and Variants Thereof

In some embodiments, a polymer included in compositions comprising a polymeric biomaterial(s) preparation described herein may be or comprises a poloxamer. Poloxamer is typically a block copolymer comprising a hydrophobic chain of polyoxypropylene (e.g., polypropylene glycol, PPG, and/or poly(propylene oxide), PPO) flanked by two hydrophilic chains of polyoxyethylene (e.g., polyethylene glycol, PEG, and/or poly(ethylene oxide), PEO). Poloxamers are known by the trade names Synperonic, Pluronic, and/or Kolliphor. Generally, poloxamers are non-ionic surfactants, which in some embodiments may have a good solubilizing capacity, low toxicity, and/or high compatibility with cells, body fluids, and a wide range of chemicals.

In some embodiments, a poloxamer for use in accordance with the present disclosure may be a poloxamer known in the art. For example, as will be understood by a skilled person in the art, poloxamers are commonly named with the letter P (for poloxamer) followed by three digits: the first two digits multiplied by 100 give the approximate molecular mass of the polyoxypropylene chain, and the last digit multiplied by 10 gives the percentage polyoxyethylene content. By way of example only, P407 refers to a poloxamer with a polyoxypropylene molecular mass of 4000 g/mol and a 70% polyoxyethylene content). A skilled person in the art will also understand that for the Pluronic and Synperonic tradenames, coding of such poloxamers starts with a letter to define its physical form at room temperature (e.g., L=liquid, P=paste, F=flake (solid)) followed by two or three digits, wherein the first digit (two digits in a three-digit number) in the numerical designation, multiplied by 300, indicates the approximate molecular weight of the polyoxypropylene chain; and the last digit, multiplied by 10, gives the percentage polyoxyethylene content. By way of example only, L61 refers to a liquid preparation of poloxamer with a polyoxypropylene molecular mass of 1800 g/mol and a 10% polyoxyethylene content. In addition, as will be apparent to a skilled artisan, poloxamer 181 (P181) is equivalent to Pluronic L61 and Synperonic PE/L61.

In some embodiments, a poloxamer that may be included in compositions comprising a polymeric biomaterial(s) preparation described herein may be or comprise Poloxamer 124 (e.g., Pluronic L44 NF), Poloxamer 188 (e.g., Pluronic F68NF), Poloxamer 181 (e.g., Pluronic L61), Poloxamer 182 (e.g., Pluronic L62), Poloxamer 184 (e.g., Pluronic L64), Poloxamer 237 (e.g., Pluronic F87 NF), Poloxamer 338 (e.g., Pluronic F108 NF), Poloxamer 331 (e.g., Pluronic L101), Poloxamer 407 (e.g., Pluronic F127 NF), or combinations thereof. In some embodiments, a provided polymeric biomaterial(s) preparation can comprise at least two or more different poloxamers. Additional poloxamers as described in Table 1 of Russo and Villa “Poloxamer Hydrogels for Biomedical Applications” Pharmaceutics (2019) 11 (12): 671, the contents of which are incorporated herein by reference for the purposes described herein, may be also useful for polymeric biomaterial(s) preparations described herein.

In some embodiments, a poloxamer that may be included in compositions comprising a polymeric biomaterial(s) preparation described herein may be or comprise Poloxamer 407 (P407). In some embodiments, P407 is a triblock poloxamer copolymer having a hydrophobic PPO block flanked by two hydrophilic PEO blocks. The approximate length of the two PEO blocks is typically 101 repeat units, while the approximate length of the PPO block is 56 repeat units. In some embodiments, P407 has an average molecular weight of approximately 12,600 Da of which approximately 70% corresponds to PEO. In some embodiments, P407 can readily self-assemble to form micelles dependent upon concentration and ambient temperature. Without wishing to be bound by a particular theory, dehydration of hydrophobic PPO blocks combined with hydration of PEO blocks may lead to formation of spherical micelles, and subsequent packing of the micellar structure results in a 3D cubic lattice that constitutes the main structure of poloxamer hydrogels. They are also non-toxic, and stable, and are therefore suitable for use as controlled release of therapeutic agents. As appreciated by one of ordinary skill in the art, P407 concentrations in hydrogel formulations based on binary poloxamer/water mixtures are typically in the range from 16-20 w/v %, with a value of approximately 18% w/v most frequently used. See, e.g., Pereia et al. “Formulation and Characterization of Poloxamer 407®: Thermoreversible Gel Containing Polymeric Microparticles and Hyaluronic Acid” Quim. Nova, Vol. 36, No. 8, 1121-1125 (2013), the contents of which are incorporated herein by reference in their entirety for purposes described herein.

In some embodiments, a poloxamer that may be included in compositions comprising a polymeric biomaterial(s) preparation described herein may be or comprise a poloxamer as described in the International Patent Application No. PCT/US21/42110 filed Jul. 17, 2021, the entire content of which is incorporated herein by reference for purposes described herein.

In some embodiments, a provided temperature-responsive polymeric biomaterial(s) preparation comprises a first polymer component (e.g., a poloxamer as described herein) and a second polymer component that is not a poloxamer. In some embodiments, a second polymer component may be present in a provided polymeric biomaterial(s) preparation at a concentration of no more than 15% (w/w). In some embodiments, a second polymer component may be present in a provided polymeric biomaterial(s) preparation at a concentration of no more than 10% (w/w), including, e.g., at a concentration of 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, a second polymer component may be present in a provided polymeric biomaterial(s) preparation at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), at least 10% (w/w), or higher. In some embodiments, a second polymer component in a provided polymeric biomaterial(s) preparation may be present at a concentration of 0.1% (w/w) to 10% (w/w), or 0.1% (w/w) to 8% (w/w), or 0.1% (w/w) to 5% (w/w), or 1% (w/w) to 5% (w/w). In some embodiments, a second polymer component may be present in a provided polymeric biomaterial(s) preparation at a concentration of 0.5% (w/w) to 10% (w/w), or 0.5% (w/w) to 5% (w/w), or 1% (w/w) to 10% (w/w), or 1% (w/w) to 5% (w/w), or 2% to 10% (w/w).

In some embodiments, a second polymer component included in a provided polymeric biomaterial(s) preparation may be or comprise at least one, including, e.g., at least two, at least three, at least four or more biocompatible and/or biodegradable polymer components. Examples of such a biocompatible and/or biodegradable polymer component include, but are not limited to immunomodulatory polymers, carbohydrate polymers (e.g., a polymer that is or comprises a carbohydrate, e.g., a carbohydrate backbone, including, e.g., but not limited to chitosan, alginate, hyaluronic acid, and/or variants thereof), polyacrylic acid, silica gels, polyethylenimine (PEI), polyphosphazene, and/or variants thereof), cellulose, chitin, chondroitin sulfate, collagen, dextran, gelatin, ethylene-vinyl acetate (EVA), fibrin, poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycarboxybetaine (PCB), polysulfobetaine (PSB), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, starch, variants thereof, and/or combinations thereof.

In some embodiments, a second polymer component included in a provided polymeric biomaterial(s) preparation is or comprises an immunomodulatory polymer, e.g., a polymer that modulates one or more aspects of an immune response (e.g., a polymer that induces innate immunity agonism). In some embodiments, an immunomodulatory polymer may be or comprise a polymer agonist of innate immunity as described in International Patent Application No. PCT/US20/31169 filed May 1, 2020, (published as WO2020/223698A1), the entire content of which is incorporated herein by reference for purposes described herein.

In some embodiments, a second polymer component included in a provided polymeric biomaterial(s) preparation may be or comprise a carbohydrate polymer, e.g., a polymer that is or comprises a carbohydrate, e.g., a carbohydrate backbone, including, e.g., but not limited to hyaluronic acid, chitosan, and/or variants thereof.

### Exemplary Hyaluronic Acid and Variants Thereof

In some embodiments, a polymer included in compositions comprising a polymeric biomaterial(s) preparation described herein may be or comprises an anionic polymer. In some embodiments, an anionic polymer may be or comprises hyaluronic acid or a variant thereof. Hyaluronic acid (HA), also known as hyaluronan or hyaluronate, is a non-sulfated member of a class of polymers known as glycosaminoglycans (GAG) that is widely distributed in body tissues. HA is found as an extracellular matrix component of tissue that forms a pericellular coat on the surfaces of cells. In some embodiments, HA is a polysaccharide (which in some embodiments may be present as a salt, e.g., a sodium salt, a potassium salt, and/or a calcium salt) having a molecular formula of (C₁₋₄H₂₁NO₁₁) n where n can vary according to the source, isolation procedure, and/or method of determination.

In some embodiments, HA that may be useful in accordance with the present disclosure can be isolated or derived from many natural sources. For example, in some embodiments, HA can be isolated or derived from, including, e.g., human umbilical cord, rooster combs, and/or connective matrices of vertebrate organisms. In some embodiments, HA can be isolated or derived from a capsular component of bacteria such as Streptococci. See, e.g., Kendall et al, (1937), Biochem. Biophys. Acta, 279, 401-405. In some embodiments, HA and/or variants thereof can be produced via microbial fermentation. In some embodiments, HA and/or variants thereof may be a recombinant HA or variants thereof, for example, produced using Gram-positive and/or Gram-negative bacteria as a host, including, e.g., but not limited to Bacillus sp., Lactococcos lactis, Agrobacterium sp., and/or Escherichia coli.

As discussed in the International Patent Application No. PCT/US20/31169 filed May 1, 2020, biological activities of HA differ, depending on its molecular weight—for example, high molecular weight HA (high MW HA) can possess anti-inflammatory or immunosuppressive activities, while low molecular weight HA (low MW HA) may exhibit pro-inflammatory or immunostimulatory behaviors. See, e.g., Gao et al. “A low molecular weight hyaluronic acid derivative accelerates excisional wound healing by modulating pro-inflammation, promoting epithelialization and neovascularization, and remodeling collagen” Int J. Mol Sci (2019) 20:3722; Cyphert et al. “Size Matters: Molecular Weight Specificity of Hyaluronan Effects in Cell Biology.” Int. J. Cell Biol. (2015) 2015:563818; Dicker et al. “Hyaluronan: A simple polysaccharide with diverse biological functions” Acta Biomater. (2014) 10:1558-1570; Aya and Stern “Hyaluronan in wound healing: Rediscovering a major player.” Wound Repair Regen. (2014) 22:579-593; and Frenkel “The role of hyaluronan in wound healing” Int. Wound J. (2014) 11:159-163, the entire contents of each of which are incorporated herein by reference in their entirety for the purposes described herein. Accordingly, in some embodiments, HA or variants thereof that may be included in compositions comprising a polymeric biomaterial(s) preparation described herein can have a low molecular weight, for example, an average molecular weight of 500 kDa or less, including, e.g., 450 kDa, 400 kDa, 350 kDa, 300 kDa, 250 kDa, 200 kDa, 150 kDa, 100 kDa, 50 kDa, or less. In some embodiments, HA or variants thereof that may be included in compositions comprising a polymeric biomaterial(s) preparation described herein may have an average molecular weight of about 100 kDa to about 150 kDa. In some embodiments, HA or variants thereof that may be included in compositions comprising a polymeric biomaterial(s) preparation described herein may have an average molecular weight of about 300 kDa to about 400 kDa. In some embodiments, compositions comprising a polymeric biomaterial(s) preparation described herein may comprise a poloxamer (e.g., ones described herein) and low molecular weight HA or variants thereof in the absence of an immunomodulatory payload may be useful for inducing innate immunity agonism.

In some embodiments, HA or variants thereof that may be included in compositions comprising a polymeric biomaterial(s) preparation described herein can have a high molecular weight, for example, an average molecular weight of greater than 500 kDa or higher, including, e.g., 550 kDa, 600 kDa, 650 kDa, 700 kDa, 750 kDa, 800 kDa, 850 kDa, 900 kDa, 950 kDa, 1 MDa, 1.1 MDa, 1.2 MDa, 1.3 MDa, 1.4 MDa, 1.5 MDa, 1.6 MDa, 1.7 MDa, 1.8 MDa, 1.9 MDa, 2 MDa, 2.5 MDa, 3 MDa, 3.5 MDa, 4 MDa, 4.5 MDa, or higher. In some embodiments, HA or variants thereof that may be useful in accordance with the present disclosure may have an average molecular weight of about 600 kDa to about 900 kDa. In some embodiments, HA or variants thereof that may be useful in accordance with the present disclosure may have an average molecular weight of about 1 MDa to about 3 MDa. In some embodiments, compositions comprising a polymeric biomaterial(s) preparation described herein may comprise a poloxamer (e.g., ones described in U.S. Provisional Patent Application 63/053,488 filed Jul. 17, 2020, which is incorporated herein by reference in its entirety for any purpose) and high molecular weight HA or variants thereof in the absence of an immunomodulatory payload may be useful for resolving inflammation (e.g., immunosuppressive inflammation).

In some embodiments, compositions comprising a polymeric biomaterial(s) preparation described herein comprises a hyaluronic acid variant. In some embodiments, a hyaluronic acid variant is water-soluble. In some embodiments, a hyaluronic acid variant may be a chemically modified hyaluronic acid, e.g., in some embodiments, hyaluronic acid is esterified. Examples of chemical modifications to hyaluronic acid include, but are not limited to, addition of thiol, haloacetate, butanediol, diglycidyl, ether, dihydrazide, aldehyde, glycan, and/or tyramine functional groups. Additional hyaluronic acid modifications and variants are known in the art. See e.g., Highley et al., “Recent advances in hyaluronic acid hydrogels for biomedical applications” Curr Opin Biotechnol (2016) Aug 40:35-40; Burdick & Prestwich, “Hyaluronic acid hydrogels for biomedical applications” Advanced Materials (2011); Prestwhich, “Hyaluronic acid-based clinical biomaterials derived for cell and molecule delivery in regenerative medicine” J. Control Release (2011) October 30; 155 (2): 193-199; each of which are incorporated herein by reference in their entirety for the purposes described herein.

In some embodiments, compositions comprising a polymeric biomaterial(s) preparation described herein comprising HA also comprise at least one poloxamer. As discussed in the U.S. Provisional Patent Application 63/053,488 filed Jul. 17, 2020, which is incorporated herein by reference in its entirety for any purpose, combinations of HA and poloxamer may be particularly efficacious for the purposes described herein.

In certain embodiments, hyaluronic acid can be cross-linked by attaching thiols (EXTRACEL®, HYSTEM®), methacrylates, hexadecylamides (HYMOVIS®), and tyramines (CORGEL®). Hyaluronic acid can also be cross-linked directly with formaldehyde (HYLAN-A®) or with divinylsulfone (HYLAN-B®).

In certain embodiments, hyaluronic acid comprises thiol-modified hyaluronic acid and a cross-linking agent. In certain embodiments, the polymer network or hydrogel comprises thiol-modified hyaluronic acid (e.g., GLYCOSIL®), and a thiol-reactive PEGDA cross-linker (e.g., EXTRALINK®). In certain embodiments, the thiol-modified hyaluronic acid and the thiol-reactive PEGDA cross-linker are combined to form a cross-linked polymer network or hydrogel useful in the compositions described herein.

In certain embodiments, the amount and concentration of thiol-modified hyaluronic acid, thiol-reactive hyaluronic acid, and cross-linking agent can be adjusted to provide compositions with desired physical properties, such as having a storage modulus of about 500 Pa to about 3000 Pa.

### Exemplary Alginate and Variants Thereof

In certain embodiments, compositions comprising at least one polymeric biomaterial as described herein comprise alginate. Alginate, in its non-salt form is known as alginic acid and/or algin. Alginate is a polysaccharide distributed widely in the cell walls of brown algae that is hydrophilic and forms a viscous gum when hydrated. Metals such as sodium and calcium are utilized to create alginates. Alginic acid has a linear structure of heteropolysaccharide. In general, alginic acid is composed of d-mannuronic acid and l-guluronic acid. Alginic acid exists as a salt, sodium alginate, in many market places. Alginates have the ability to form gels in the presence of bipolar ions (such as calcium ions) because the carboxyl groups are in their polymer chains. Due to the porous structure and high water-absorption capacity of calcium alginate gel, it has previously been proposed for use in wound dressing and/or the immobilization of cells.

In certain embodiments, compositions comprising at least one polymeric biomaterial are comprised of the polymeric biomaterial alginate. In certain embodiments, the polymeric biomaterial comprises cross-linked alginate. In certain embodiments, the polymeric biomaterial is alginate. In certain embodiments, the polymeric biomaterial is cross-linked alginate. In certain embodiments, the polymeric biomaterial can form a polymer network or hydrogel, wherein the polymeric biomaterial comprises alginate. In certain embodiments, the polymer network or hydrogel comprises cross-linked alginate. In certain embodiments, the polymer network or hydrogel is alginate. In certain embodiments, the polymer network or hydrogel is cross-linked alginate. In certain embodiments, the polymeric biomaterial does not comprise alginate. In certain embodiments, the polymeric biomaterial is not alginate. In certain embodiments, the polymer network or hydrogel is not alginate. In certain embodiments, the polymer network or hydrogel does not comprise alginate.

In certain embodiments, alginate can be cross-linked ionically by adding a salt that promotes cross-linking (e.g., calcium chloride).

In certain embodiments, alginate comprises alginate and a cross-linking agent (e.g., calcium chloride). In certain embodiments, the polymer network or hydrogel comprises alginate and a cross-linking agent (e.g., calcium chloride). In certain embodiments, the alginate and the calcium chloride (e.g., ionic cross-linker) are combined to form a cross-linked polymer network or hydrogel useful in the compositions described herein.

In certain embodiments, the amount and concentration of alginate and calcium chloride can be adjusted to provide compositions with desired physical properties, such as having a storage modulus of about 500 Pa to about 3000 Pa

### Exemplary Chitosan and Variants Thereof

In some embodiments, compositions comprising a polymeric biomaterial(s) preparation described herein may be or comprise a cationic polymer. In some embodiments, a cationic polymer may be or comprise chitosan or a variant thereof. Examples of chitosan and/or variants thereof that can be included in a polymer combination preparations described herein include, but are not limited to chitosan, chitosan salts (e.g., chitosan HCl, chitosan chloride, chitosan lactate, chitosan acetate, chitosan glutamate), alkyl chitosan, aromatic chitosan, carboxyalkyl chitosan (e.g., carboxymethyl chitosan), hydroxyalkyl chitosan (e.g., hydroxypropyl chitosan, hydroxyethyl chitosan), aminoalkyl chitosan, acylated chitosan, phosphorylated chitosan, thiolated chitosan, quaternary ammonium chitosan (e.g., N-(2-hydroxyl) propyl-3-trimethyl ammonium chitosan chloride), guanidinyl chitosan, chitosan oligosaccharide, glycated chitosan (e.g., N-dihydrogalactochitosan), and variants or combinations thereof. In some embodiments, compositions comprising a polymeric biomaterial(s) preparation described herein comprises carboalkyl chitosan (e.g., carboxymethyl chitosan).

Those skilled in the art will appreciate that, in some cases, chitosan and/or variants thereof can be produced by deacetylation of chitin. In some embodiments, chitosan or variants thereof included in a polymer combination preparation comprising poloxamer (e.g., as described herein) is characterized by degree of deacetylation (i.e., percent of acetyl groups removed) of at least 70% or above, including, e.g., at least 75%, at least 80%, at least 85%, at least 90%, at least 95%, or higher (including up to 100%). In some embodiments, a chitosan or variants thereof is characterized by degree of deacetylation of no more than 99%, no more than 95%, no more than 90%, no more than 85%, no more than 80%, no more than 75% or lower. Combinations of the above-mentioned ranges are also possible. For example, a chitosan or variants thereof may be characterized by degree of deacetylation of 80%-95%, 70%-95%, or 75%-90%. As will be recognized by one of those skilled in the art, degree of deacetylation (% DA) can be determined by various methods known in the art, e.g., in some cases, by NMR spectroscopy.

In some embodiments, chitosan or variants thereof included in compositions comprising a polymeric biomaterial(s) preparation described herein may have an average molecular weight of at least 5 kDa or higher, including, e.g., at least 10 kDa or higher, including, e.g., at least 20 kDa, at least 30 kDa, at least 40 kDa, at least 50 kDa, at least 60 kDa, at least 70 kDa, at least 80 kDa, at least 90 kDa, at least 100 kDa, at least 110 kDa, at least 120 kDa, at least 130 kDa, at least 140 kDa, at least 150 kDa, at least 160 kDa, at least 170 kDa, at least 180 kDa, at least 190 kDa, at least 200 kDa, at least 210 kDa, at least 220 kDa, at least 230 kDa, at least 240 kDa, at least 250 kDa, at least 260 kDa, at least 270 kDa, at least 280 kDa, at least 290 kDa, at least 300 kDa, at least 350 kDa, at least 400 kDa, at least 500 kDa, at least 600 kDa, at least 700 kDa, or higher. In some embodiments, chitosan or variants thereof included in compositions comprising a polymeric biomaterial(s) preparation described herein may have an average molecular weight of no more than 750 kDa or lower, including, e.g., no more than 700 kDa, no more than 600 kDa, no more than 500 kDa, no more than 400 kDa, no more than 300 kDa, no more than 200 kDa, no more than 100 kDa, no more than 50 kDa, or lower. Combinations of the above-mentioned ranges are also possible. For example, in some embodiments, chitosan or variants thereof included in compositions comprising a polymeric biomaterial(s) preparation described herein is characterized by an average molecular weight of 10 kDa to 700 kDa, or 20 kDa to 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in compositions comprising a polymeric biomaterial(s) preparation described herein is characterized by an average molecular weight of 20 kDa to 700 kDa, or 30 kDa to 500 kDa. As noted herein, an average molecular weight may be a number average molecular weight, weight average molecular weight, or peak average molecular weight.

In some embodiments, chitosan or variants thereof included in compositions comprising a polymeric biomaterial(s) preparation described herein is characterized by a molecular weight distribution in a range of 10 kDa to 700 kDa, or 20 kDa or 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in compositions comprising a polymeric biomaterial(s) preparation described herein is characterized by a molecular weight distribution in a range of 20 kDa to 700 kDa, or 30 kDa to 500 kDa.

In some embodiments, chitosan or variants thereof included in compositions comprising a polymeric biomaterial(s) preparation described herein may be characterized by a viscosity of no more than 3500 mPa·s or lower, including, e.g., no more than 3000 mPa·s, no more than 2500 mPa·s, no more than 2000 mPa·s, no more than 1500 mPa·s, no more than 1000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, chitosan or variants thereof may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1000 mPa·s, at least 1500 mPa·s, at least 2000 mPa·s, at least 2500 mPa·s, or higher. Combinations of the above-mentioned ranges are also possible. For example, in some embodiments, such a viscous polymer solution of or comprising chitosan or variants thereof may be characterized by a viscosity of 5 mPa·s to 3000 mPa·s, or 5 mPa·s to 300 mPa·s, 5 mPa·s to 200 mPa′s, or 20 mPa·s to 200 mPa′s, or 5 mPa·s to 20 mPa·s. In some embodiments, viscosity of chitosan or variants thereof described herein is measured at 1% in 1% acetic acid at 20° C.

In some embodiments, a compositions comprising a polymeric biomaterial(s) preparation described herein comprises at least one or more (e.g., 1, 2, 3 or more) chitosan and/or variants thereof (including, e.g., modified chitosan and/or salts of chitosan or modified chitosan such as a chloride salt or a glutamate salt). For example, in some embodiments, chitosan and/or variants thereof (including, e.g., modified chitosan and/or salts of chitosan or modified chitosan such as a chloride salt or a glutamate salt) may be characterized by degree of deacetylation of 70%-95%, or 75%-90%, or 80%-95%, or greater than 90%. In some embodiments, chitosan and/or variants thereof (including, e.g., modified chitosan and/or salts of chitosan or modified chitosan such as a chloride salt or a glutamate salt) may be characterized by an average molecular weight of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., modified chitosan and/or salts of chitosan or modified chitosan such as a chloride salt or a glutamate salt) may be characterized by a molecular weight distribution in the range of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., salts thereof such as a chloride salt or a glutamate salt) may be characterized by a viscosity ranging from 5 to 3000 mPa·s, or 5 to 300 mPa·s, or 20 to 200 mPa·s. In some embodiments, such chitosan and/or variants thereof (including, e.g., salts thereof such as a chloride salt or a glutamate salt) may be or comprise PROTASAN™ UltraPure chitosan chloride and/or chitosan glutamate salt (e.g., obtained from NovoMatrix®, which is a business unit of FMC Health and Nutrition (now a part of Du Pont; Product No. CL 113, CL 114, CL 213, CL 214, G 113, G 213, G 214). In some embodiments, such chitosan and/or variants thereof (including, e.g., salts thereof such as a chloride salt or a glutamate salt) may be or comprise chitosan, chitosan oligomers, and/or variants thereof (including, e.g., Chitosan HCl, carboxymethyl chitosan, chitosan lactate, chitosan acetate), e.g., obtained from Heppe Medical Chitosan GMBH (e.g., Chitoceuticals® or Chitoscience®).

In some embodiments, chitosan or variants thereof included in compositions comprising a polymeric biomaterial(s) preparation described herein is or comprises carboxyalkyl chitosan (e.g., carboxymethyl chitosan) that is characterized by at least one or all of the following characteristics: (1) degree of deacetylation of 80%-95%; (ii) an average molecular weight of 30 kDa to 500 kDa; or a molecular weight distribution of 30 kDa to 500 kDa; and (iii) a viscosity ranging from 5 to 300 mPa·s.

In some embodiments, chitosan or variants thereof included in compositions comprising a polymeric biomaterial(s) preparation described herein is or comprises a variant of chitosan (e.g., as described herein). In some embodiments, such a variant of chitosan may include chemical modification(s) of one or more chemical moieties, e.g., hydroxyl and/or amino groups, of the chitosan chains. In some embodiments, such a variant of chitosan is or comprises a modified chitosan such as, e.g., but not limited to a glycated chitosan (e.g., chitosan modified by addition of one or more monosaccharide or oligosaccharide side chains to one or more of its free amino groups). Exemplary glycated chitosan that are useful herein include, e.g., but are not limited to ones described in U.S. Pat. Nos. 5,747,475, 6,756,363, WO 2013/109732, US 2018/0312611, and US 2019/0002594, the contents of each of which are incorporated herein by reference for the purposes described herein.

In some embodiments, chitosan or variants thereof included in compositions comprising a polymeric biomaterial(s) preparation described herein is or comprises chitosan conjugated with a polymer that increases its solubility in aqueous environment (e.g., a hydrophilic polymer such as polyethylene glycol).

In some embodiments, chitosan or variants thereof included in compositions comprising a polymeric biomaterial(s) preparation described herein is or comprises thiolated chitosan. Various modifications to chitosans, e.g., but not limited to carboxylation, PEGylation, galactosylation (or other glycations), and/or thiolation are known in the art, e.g., as described in Ahmadi et al. Res Pharm Sci., 10 (1): 1-16 (2015), the contents of which are incorporated herein by reference for the purposes described herein. Those skilled in the art reading the present disclosure will appreciate that other modified chitosans can be useful for a particular application in which a method is being practiced.

In some embodiments, compositions comprising a polymeric biomaterial(s) preparation described herein comprising chitosan or variant thereof also comprise at least one poloxamer. As discussed in the U.S. Provisional Patent Application 63/053,488 filed Jul. 17, 2020, which is incorporated herein by reference in its entirety for any purpose, combinations of chitosan and poloxamer may be particularly efficacious for the purposes described herein.

Polynucleotide Agent(s) that Encode or Regulate an Immunomodulatory Polypeptide

Provided compositions comprise at least one nucleic acid or polynucleotide agent. In certain embodiments, provided nucleic acid(s) may be deoxyribonucleic acid(s) and/or ribonucleic acid(s). In certain embodiments, provided nucleic acid(s) may be chemically modified in ways that may alter stability, function, or other characteristics. In certain embodiments, compositions comprise at least one nucleic acid or polynucleotide agent that acts to directly regulate and/or modify the immune system, and/or encodes a molecule that regulates the immune system or regulates an immunomodulatory peptide. In some embodiments, the nucleic acid acts to encode or modulate an immunomodulatory peptide which comprises a cytokine, chemokine, antibody, innate immune system modulator, adaptive immune system modulator, pro-inflammatory peptide, anti-inflammatory peptide, growth factor, hormone, and/or any domain, combination, or hybrid construct of these.

In certain embodiments, provided compositions comprise at least one polynucleotide agent that regulates or encodes a molecule that induces innate immunity by activating a pattern recognition receptor. In certain embodiments, an activator of innate immune response is a ligand of a pattern recognition receptor (PRR). In certain embodiments, an activator of innate immune response is an agonist of a pattern recognition receptor (PRR). In certain embodiments, an activator of the innate immune response may be recognized by Toll-like receptors (TLR), Nucleotide-binding oligomerization domain-like receptors (NLR), C-type lectin receptors (CLR), and/or RIG-1 like receptors (RLR).

In certain embodiments, provided compositions may comprise at least one nucleic acid which comprises a sequence encoding a cytokine. Alternatively, in some embodiments, compositions comprising at least one nucleic acid may further comprise a cytokine.

Cytokines are a broad category of small proteins (˜5-20 kDa) that are important in cell signaling. Their release has an effect on the behavior of cells around them. Cytokines may induce innate immunity and/or adaptive immunity. Cytokines are involved in autocrine signaling, paracrine signaling, and endocrine signaling as immunomodulating agents. Cytokines include chemokines, interferons, interleukins, lymphokines, and tumor necrosis factors. Cytokines are produced by a broad range of cells, including immune cells, such as macrophages, B lymphocytes, T lymphocytes, and mast cells, as well as endothelial cells, fibroblasts, and various stromal cells. They act through receptors and play an important role in the immune system. Cytokines modulate the balance between humoral and cell-based immune responses, and they regulate the maturation, growth, and responsiveness of particular cell populations. Some cytokines enhance or inhibit the action of other cytokines in complex ways. Cytokines are important in host responses to infection, immune responses, inflammation, trauma, sepsis, cancer, and reproduction.

Furthermore, it is currently known in the art that the method of delivery, dosing and scheduling, and toxicity-related issues must be addressed to enable the immune-stimulating function of many cytokines and chemokines to be fully exploited.

In certain embodiments, provided compositions comprise at least one nucleic acid encoding a cytokine, and/or at least one cytokine, wherein the included cytokine is and/or nucleotide sequence encodes IL-1, IL-la, IL-1β, IL-2, an IL-2 superkine, IL-6, IL-7, IL-9, AM0010, IL-12, IL-15, an IL-15 superagonist, ALT-803, NIZ985, IL-16, IL-18, IL-21, an IL-21 superagonist, denenicokin, an IL-21 superagonist antibody, IFN-α, IFN-β, IFN-γ, TNF-α, GM-CSF, a cytokine fusion, RG7461, RG7813, M9241, NKTR-214, NKTR-255, BMS-982470, BG-00001, Flt3L, or CDX-301.

In certain embodiments, provided compositions comprise at least one nucleic acid encoding a cytokine, and/or at least one cytokine, wherein the included cytokine is and/or nucleotide sequence encodes ALT-803, NIZ985, denenicokin, RG7461, RG7813, M9241, IFN-α, IFN-β, or IFN-γ.

In certain embodiments, provided compositions comprise at least one nucleic acid encoding a cytokine, and/or at least one cytokine, wherein the included cytokine is and/or nucleotide sequence encodes an IL-15 superagonist and/or IL-21.

In certain embodiments, provided compositions comprise at least one nucleic acid encoding a cytokine, and/or at least one cytokine, wherein the included cytokine is and/or nucleotide sequence encodes an IL-15 superagonist, IL-21, IFN-α, IFN-β, IFN-γ, CCL4, CCL5, CXCL9, or CXCL10. In certain embodiments, an encoded sequence and/or cytokine is an IL-15 superagonist, IFN-α, IFN-β, or IFN-γ.

IL-15 (Interleukin 15) is a cytokine with structural similarity to IL-2 and is secreted by mononuclear phagocytes following infection by virus(es). IL-15 induces cell proliferation of natural killer cells, cells whose principal role is to kill virally infected cells. The combination of IL-15 with soluble IL-15Ra generates a complex termed IL-15 superagonist (IL-15sa) that possesses greater biological activity than IL-15 alone. IL-15sa is an antitumor and antiviral agent because of its ability to selectively expand NK and memory CD8+T (mCD8+T) lymphocytes.

In certain embodiments, provided compositions comprise at least one nucleic acid encoding a cytokine, and/or at least one cytokine, wherein the included cytokine is and/or nucleotide sequence encodes an IL-15 superagonist known as ALT-803. ALT-803 is thought to induce memory CD8+ T cells to proliferate, upregulate receptors involved in innate immunity, secrete interferon-γ, and acquire the ability to kill malignant cells in the absence of antigenic stimulation. Thus, ALT-803 can promote the expansion and activation of memory CD8+ T cells while converting them into innate immune effector cells that exhibit robust antineoplastic activity. ALT-803 is a fusion protein of an IL-15 mutant and the IL-15Ra/Fc complex that has recently entered clinical trials as a direct immunomodulatory agent. ALT-803 exhibits >25-fold enhancement in biological activity as compared to IL-15.

In certain embodiments, provided compositions comprise at least one nucleic acid encoding a cytokine, and/or at least one cytokine, wherein the included cytokine is and/or nucleotide sequence encodes NIZ985 (hetIL-15). Studies have demonstrated that hetIL-15 administration can promote an increase of tumor infiltration and persistence of CD8+ T cells, including tumor-specific T cells, and result in an increased CD8+/Treg ratio. Tumor-resident CD8+ T cells show features of effector cells and are characterized by increased proliferation (Ki67+) and high cytotoxic potential (Granzyme B+). In the absence of hetIL-15, the smaller population of tumor-infiltrating T cells exhibit high levels of the exhaustion marker PD-1, potentially limiting their anti-cancer effectiveness. Provision of hetIL-15 can result in a significant decrease in lymphocyte expression of PD-1, alleviating one potential mechanism for the exhaustion phenotype. Preclinical cancer studies support the use of hetIL-15 in tumor immunotherapy approaches to promote the development of anti-tumor responses by favoring effector over regulatory cells.

In certain embodiments, provided compositions comprise at least one nucleic acid encoding a cytokine, and/or at least one cytokine, wherein the included cytokine is and/or nucleotide sequence encodes interferon α (IFN-α). The IFN-α proteins are produced by leukocytes. They are mainly involved in innate immune response against viral infection.

In certain embodiments, provided compositions comprise at least one nucleic acid encoding a cytokine, and/or at least one cytokine, wherein the included cytokine is and/or nucleotide sequence encodes interferon β (IFN-β). IFN-β comprises proteins produced by fibroblasts and is involved in innate immune response. IFN-β stimulates both macrophages and NK cells to elicit an anti-viral response, and are also active against tumors. In mice, IFN-β inhibits immune cells to produce growth factors, thereby slowing tumor growth, and inhibits other cells from producing vessel producing growth factors, thereby blocking tumor angiogenesis and hindering the tumor from connecting into the blood vessel system.

In certain embodiments, provided compositions comprise at least one nucleic acid encoding a cytokine, and/or at least one cytokine, wherein the included cytokine is and/or nucleotide sequence encodes interferon γ (IFN-γ). IFN-γ, or type II interferon, is a cytokine that is useful for innate and adaptive immunity. IFN-γ is an important activator of macrophages and inducer of Class II major histocompatibility complex (MHC) molecule expression. The in vitro study of IFN-γ in cancer cells is extensive and results indicate anti-proliferative activity of IFN-γ leading to growth inhibition or cell death, generally induced by apoptosis but sometimes by autophagy. Clinical administration of IFN-γ has resulted in improved survival for patients with ovarian, bladder, and melanoma cancers.

In certain embodiments, provided compositions comprise at least one nucleic acid encoding a chemokine, and/or at least one chemokine. Chemokines are a family of small cytokines. The major role of chemokines is to act as a chemoattractant to guide the migration of cells. Some chemokines control cells of the immune system during processes of immune surveillance, such as directing lymphocytes to the lymph nodes so they can screen for invasion of pathogens by interacting with antigen-presenting cells residing in these tissues. These are known as homeostatic chemokines and are produced and secreted without any need to stimulate their source cell(s). Some chemokines play a role in development, promote angiogenesis (the growth of new blood vessels), or guide cells to tissues that provide specific signals critical for cellular maturation. Other chemokines are inflammatory and are released from a wide variety of cells in response to bacteria, viruses, and agents that cause physical damage, such as silica or the urate crystals that occur in gout. Their release is often stimulated by pro-inflammatory cytokines, such as interleukin 1. Inflammatory chemokines function mainly as chemoattractants for leukocytes, recruiting monocytes, neutrophils, and other effector cells from the blood to sites of infection or tissue damage. Certain inflammatory chemokines activate cells to initiate an immune response or promote wound healing. They are released by many different cell types and serve to guide cells of both the innate immune system and adaptive immune system.

Furthermore, it is currently known in the art that the method of delivery, dosing and scheduling, and toxicity-related issues must be addressed to enable the immune-stimulating function of many chemokines to be fully exploited. Thus, in some embodiments, provided compositions may solve many of the known barriers to chemokine exploitation for immunotherapy.

In certain embodiments, provided compositions comprise at least one nucleic acid encoding a chemokine, and/or at least one chemokine, wherein the included chemokine is and/or nucleotide sequence encodes CCL1, CCL2, CCL3, CCL4, CCL5, CCL17, CCL19, CCL21, CCL22, CXCL9, CXCL10, CXCL11, CXCL13, CXCL16, or CX3CL1.

In certain embodiments, provided compositions comprising at least one nucleic acid may comprise nucleic acids encoding a modulator (e.g., inhibitor) of the pro-inflammatory pathway. In some embodiments, the nucleic acid encoded inhibitor of a pro-inflammatory pathway comprises an antibody. In some embodiments, provided compositions may comprise more than one inhibitor of a pro-inflammatory pathway. In some embodiments, an inhibitor of a pro-inflammatory pathway may prevent recruitment of immunosuppressive cells. In some embodiments, an inhibitor of a pro-inflammatory pathway may prevent acute inflammation. In some embodiments, an inhibitor of a pro-inflammatory pathway may inhibit, for example, an immune response that induces inflammation, including, e.g., production of one or more pro-inflammatory cytokines (e.g., TNF-alpha, IL-1beta, and/or IL-6), increased activity and/or proliferation of Th1 cells, recruitment of myeloid cells, etc. For example, in some embodiments, an inhibitor of a pro-inflammatory pathway can be an inhibitor of IL-1beta. In some embodiments, an inhibitor of a pro-inflammatory pathway can be an inhibitor of IL-6.

In certain embodiments, provided compositions may comprise at least one nucleic acid which acts as or encodes an inhibitor of the proinflammatory pathway. In some embodiments an inhibitor of a pro-inflammatory pathway is an inhibitor of proinflammatory immune response mediated by p38 mitogen-activated protein kinase (MAPK) pathway as described herein.

In certain embodiments, provided compositions may comprise at least one nucleic acid which acts as or encodes an inhibitor of a pro-inflammatory pathway that prevents recruitment of immunosuppressive cells. In certain embodiments, an inhibitor of a pro-inflammatory pathway is an inhibitor, antagonist, or partial agonist of CCR2, CCR5, CXCR2, CXCR4, CXCL12, or CCL2. In certain embodiments, an inhibitor of a pro-inflammatory pathway is an inhibitor, antagonist, or partial agonist of CCR5, CXCR2, CXCL12, or CCL2.

In certain embodiments, provided compositions may comprise at least one nucleic acid which acts as or encodes an inhibitor of a pro-inflammatory pathway, and an optional further inhibitor of a pro-inflammatory pathway which is an inhibitor, antagonist, or partial agonist of CCR2. In certain embodiments, CCR2 is related to the p38 MAPK pathway (e.g., as described in Montague, et al., J. Inflammation 2018, 15:101; and Xu, et al., Am. J. Transl. Res. 2017, 9, 2878-2890; which are each incorporated herein by reference for the purpose described herein). In certain embodiments, an optional inhibitor, antagonist, or partial agonist of CCR2 is PF-04136309, CCX872-B, or plozalizumab. In certain embodiments, an inhibitor of a pro-inflammatory pathway is PF-04136309, CCX872-B, or plozalizumab. In certain embodiments, an inhibitor of a pro-inflammatory pathway is not an inhibitor, antagonist, or partial agonist of CCR2. In certain embodiments, the inhibitor of a pro-inflammatory pathway is not PF-04136309.

In certain embodiments, provided compositions may comprise at least one nucleic acid which acts as or encodes an inhibitor of a pro-inflammatory pathway, wherein the inhibitor of a pro-inflammatory pathway is an inhibitor, antagonist, or partial agonist of CCR5. In certain embodiments, CCR5 is related to the p38 MAPK pathway (e.g., as described in Lei, et al., Biochem. Biophys. Res. Commun. 2005, 329, 610-615; and Manes, et al., J. Exp. Med. 2003, 198, 1381-1389; which are each incorporated herein by reference for the purpose described herein). In certain embodiments, the nucleic acid encoded inhibitor, antagonist, or partial agonist of CCR5 is optionally coupled with maraviroc, DAPTA, GSK706769, INCB009471, GW873140, Vicriviroc, or PRO 140.

In certain embodiments, provided compositions may comprise at least one nucleic acid which acts as or encodes an inhibitor of a pro-inflammatory pathway, wherein the inhibitor of a pro-inflammatory pathway is an inhibitor, antagonist, or partial agonist of CCR2 and/or CCR5. In certain embodiments, the nucleic acid encoded inhibitor, antagonist, or partial agonist of CCR2 and CCR5 is optionally coupled with PF-04634817, cenicriviroc, or BMS-813160.

In certain embodiments, provided compositions may comprise at least one nucleic acid which acts as or encodes an inhibitor of a pro-inflammatory pathway, wherein the inhibitor of a pro-inflammatory pathway an inhibitor, antagonist, or partial agonist of CXCR2. In certain embodiments, the nucleic acid encoded inhibitor, antagonist, or partial agonist of CXCR2 is optionally coupled with danirixin, QBM076, SX-682, or SB225002.

In certain embodiments, provided compositions may comprise at least one nucleic acid which acts as or encodes an inhibitor of a pro-inflammatory pathway, wherein the inhibitor of a pro-inflammatory pathway is an inhibitor, antagonist, or partial agonist of CXCR4. In certain embodiments, CXCR4 is related to the p38 MAPK pathway (e.g., as described in Lei, et al., Biochem. Biophys. Res. Commun. 2005, 329, 610-615; and Trushin, et al., J. Immunol. 2007, 178, 4846-4853; which are each incorporated herein by reference for the purpose described herein). In certain embodiments, the nucleic acid encoded inhibitor, antagonist, or partial agonist of CXCR4 is optionally coupled with plerixafor, AMD070, AMD3465, AMD11070, LY2510924, MSX-122, TG-0054, CX-01, X4P-001, BL-8040, USL311, or SPO1A. In certain embodiments, the inhibitor of a pro-inflammatory pathway is not an inhibitor, antagonist, or partial agonist of CXCR4.

In certain embodiments, provided compositions may comprise at least one nucleic acid which acts as or encodes an inhibitor of a pro-inflammatory pathway, wherein the inhibitor of a pro-inflammatory pathway is an inhibitor, antagonist, or partial agonist of CXCL12. In certain embodiments, CXCL12 is related to the p38 MAPK pathway (e.g., as described in Gao, et al., Int. J. Clin. Exp. Pathol. 2018, 11, 3119-3125; which is incorporated herein by reference for the purpose described herein).

In certain embodiments, provided compositions may comprise at least one nucleic acid which acts as or encodes an inhibitor of a pro-inflammatory pathway, wherein the inhibitor of a pro-inflammatory pathway is an inhibitor, antagonist, or partial agonist of CCL2. In certain embodiments, CCL2 is related to the p38 MAPK pathway (e.g., as described in Cho, et al., J. Neuroimmunol. 2008, 199, 94-103; and Marra, et al., Am. J. Physiol. Gastrointest. Liver Physiol. 2004, 287, G18-26; which are each incorporated herein by reference for the purpose described herein). In certain embodiments, the nucleic acid encoded inhibitor, antagonist, or partial agonist of CCL2 is optionally coupled with bindarit.

In certain embodiments, provided compositions may comprise at least one nucleic acid which acts as or encodes an inhibitor of a pro-inflammatory pathway, wherein the inhibitor of a pro-inflammatory pathway is optionally coupled with PF-04136309, CCX872-B, plozalizumab, maraviroc, DAPTA, GSK706769, INCB009471, GW873140, Vicriviroc, PRO 140, PF-04634817, cenicriviroc, BMS-813160, danirixin, QBM076, SX-682, SB225002, plerixafor, AMD070, AMD3465, AMD11070, LY2510924, MSX-122, TG-0054, CX-01, X4P-001, BL-8040, USL311, or SPO1A.

In certain embodiments, provided compositions may comprise at least one nucleic acid which acts as or encodes an inhibitor of a pro-inflammatory pathway, wherein the inhibitor of a pro-inflammatory pathway is optionally coupled with CCX872-B, plozalizumab, maraviroc, DAPTA, GSK706769, INCB009471, GW873140, Vicriviroc, PRO 140, PF-04634817, cenicriviroc, BMS-813160, danirixin, QBM076, SX-682, SB225002, plerixafor, AMD070, AMD3465, AMD11070, LY2510924, MSX-122, TG-0054, CX-01, X4P-001, BL-8040, USL311, or SPO1A.

In certain embodiments, provided compositions may comprise at least one nucleic acid which acts as or encodes an inhibitor of a pro-inflammatory pathway, wherein the inhibitor of a pro-inflammatory pathway prevents acute inflammation. In certain embodiments, the inhibitor of a pro-inflammatory pathway is an anti-IL-la antibody, an anti-IL-1β antibody, an anti-IL-1R antibody, an IL-1 inhibitor, an anti-IL-6 antibody, an anti-IL-6R antibody, an anti-IL17 antibody, an anti-IL-17A antibody, an anti-IL-17RA antibody, an anti-IL-23/IL-12 antibody, or an anti-IL-23 antibody.

In certain embodiments, provided compositions may comprise at least one nucleic acid which encodes an inhibitor of a pro-inflammatory pathway, wherein the inhibitor of a pro-inflammatory pathway is an anti-IL-la antibody. In certain embodiments, the anti-IL-la antibody is MABp1. In certain embodiments, the inhibitor of a pro-inflammatory pathway is MABp1.

In certain embodiments, provided compositions may comprise at least one nucleic acid which encodes an inhibitor of a pro-inflammatory pathway, wherein the inhibitor of a pro-inflammatory pathway is an anti-IL-1B antibody. In certain embodiments, IL-1B is related to the p38 MAPK pathway (e.g., as described in Kulawik, et al., J. Biol. Chem. 2017, 292, 6291-6302; Rovin, et al., Cytokine 1999, 11, 118-126; Laporte, et al., Am. J. Physiol. Lung Cell Mol. Physiol. 2000, 279, L932-L941; Baldassare, et al., J. Immunol. 1999, 162, 5367-5373; and Weber, et al. Sci. Signal. 2010, 3, cm1; which are each incorporated herein by reference for the purpose described herein). In certain embodiments, the anti-IL-1β antibody is canakinumab. In certain embodiments, the inhibitor of a pro-inflammatory pathway is canakinumab.

In certain embodiments, provided compositions may comprise at least one nucleic acid which encodes an inhibitor of a pro-inflammatory pathway, wherein the inhibitor of a pro-inflammatory pathway is an anti-IL-1R antibody. In certain embodiments, IL-1R is related to the p38 MAPK pathway (e.g., as described in Weber, et al., Sci. Signal. 2010, 3, cm1; and Jain, et al., Nat. Commun. 2018, 9:3185; which are each incorporated herein by reference for the purpose described herein). In certain embodiments, the anti-IL-1R antibody is anakinra. In certain embodiments, the inhibitor of a pro-inflammatory pathway is anakinra.

In certain embodiments, provided compositions may comprise at least one nucleic acid encoding an inhibitor of a pro-inflammatory pathway, wherein the inhibitor of a pro-inflammatory pathway is an IL-1 inhibitor. In certain embodiments, the IL-1 inhibitor is rilonacept. In certain embodiments, the inhibitor of a pro-inflammatory pathway is rilonacept.

In certain embodiments, provided compositions may comprise at least one nucleic acid encoding an inhibitor of a pro-inflammatory pathway, wherein the inhibitor of a pro-inflammatory pathway is an anti-IL-6 antibody. In certain embodiments, IL-6 is related to the p38 MAPK pathway (e.g., as described in Sinfield, et al., Biochem. Biophys. Res. Commun. 2013, 430, 419-424; Suzuki, et al., FEBS Lett. 2000, 465, 23-27; and Nishikai-Yan Shen, et al., PLOS One 2017, 12, 1-17; which are each incorporated herein by reference for the purpose described herein). In certain embodiments, the anti-IL-6 antibody is olokizumab, clazakizumab, OPR-003, sirukumab, ARGX-109, FE301, or FM101. In certain embodiments, the inhibitor of a pro-inflammatory pathway is olokizumab, clazakizumab, OPR-003, sirukumab, ARGX-109, FE301, or FM101.

In certain embodiments, provided compositions may comprise at least one nucleic acid encoding an inhibitor of a pro-inflammatory pathway, wherein the inhibitor of a pro-inflammatory pathway is an anti-IL-6R antibody. In certain embodiments, the anti-IL-6R antibody is tocilizumab, sarilumab, or vobarilizumab. In certain embodiments, the inhibitor of a pro-inflammatory pathway is tocilizumab, sarilumab, or vobarilizumab.

In certain embodiments, provided compositions may comprise at least one nucleic acid encoding an inhibitor of a pro-inflammatory pathway, wherein the inhibitor of a pro-inflammatory pathway is an anti-IL-17 antibody. In certain embodiments, IL-17 is related to the p38 MAPK pathway (e.g., as described in Noubade, et al., Blood 2011, 118, 3290-3300; Roussel, et al., J. Immunol. 2010, 184, 4531-4537; and Mai, et al., J. Biol. Chem. 2016, 291, 4939-4954; which are each incorporated herein by reference for the purpose described herein). In certain embodiments, the anti-IL-17 antibody is ixekizumab, bimekizumab, ALX-0761, CJM112, CNTO 6785, LY3074828, SCH-900117, or MSB0010841. In certain embodiments, the inhibitor of a pro-inflammatory pathway is ixekizumab, bimekizumab, ALX-0761, CJM112, CNTO 6785, LY3074828, SCH-900117, or MSB0010841.

In certain embodiments, provided compositions may comprise at least one nucleic acid encoding an inhibitor of a pro-inflammatory pathway, wherein the inhibitor of a pro-inflammatory pathway is an anti-IL-17A antibody. In certain embodiments, the anti-IL17A antibody is secukinumab. In certain embodiments, the inhibitor of a pro-inflammatory pathway is secukinumab.

In certain embodiments, provided compositions may comprise at least one nucleic acid encoding an inhibitor of a pro-inflammatory pathway, wherein the inhibitor of a pro-inflammatory pathway is an anti-IL-17RA antibody. In certain embodiments, the anti-IL17RA antibody is brodalumab. In certain embodiments, the inhibitor of a pro-inflammatory pathway is brodalumab.

In certain embodiments, provided compositions may comprise at least one nucleic acid encoding an inhibitor of a pro-inflammatory pathway, wherein the inhibitor of a pro-inflammatory pathway is an anti-IL-23/IL-12 antibody. In certain embodiments, the anti-IL-23/IL-12 antibody is ustekinumab or briakinumab. In certain embodiments, the inhibitor of a pro-inflammatory pathway is ustekinumab or briakinumab.

In certain embodiments, provided compositions may comprise at least one nucleic acid encoding an inhibitor of a pro-inflammatory pathway, wherein the inhibitor of a pro-inflammatory pathway is an anti-IL-23 antibody. In certain embodiments, IL-23 is related to the p38 MAPK pathway (e.g., as described in Tang, et al., Immunology 2012, 135, 112-124; and Canavese, et al., J. Clin. Exp. Dermatol. Res. 2011, S2: 002. doi: 10.4172/2155-9554; which are each incorporated herein by reference for the purpose described herein). In certain embodiments, the anti-IL-23 antibody is tildrakizumab, BI 655066, or guselkumab. In certain embodiments, the inhibitor of a pro-inflammatory pathway is tildrakizumab, BI 655066, or guselkumab.

In certain embodiments, provided compositions may comprise at least one nucleic acid encoding an inhibitor of a pro-inflammatory pathway, wherein the inhibitor of a pro-inflammatory pathway is MABp1, canakinumab, anakinra, rilonacept, olokizumab, clazakizumab, OPR-003, sirukumab, ARGX-109, FE301, tocilizumab, sarilumab, vobarilizumab, ixekizumab, bimekizumab, sonelokimab (ALX-0761, MSB0010841), CJM112, CNTO 6785, mirikizumab (LY3074828), SCH-900117, secukinumab, brodalumab, ustekinumab briakinumab, tildrakizumab, risankizumab (BI 655066), or guselkumab.

In certain embodiments, provided compositions may comprise at least one nucleic acid which acts as or encodes an inhibitor of a pro-inflammatory pathway, wherein the inhibitor of a pro-inflammatory pathway is a TGFβR inhibitor. In certain embodiments, TGFβR is related to the p38 MAPK pathway (e.g., as described in Yu, et al., EMBO) J. 2002, 21, 3749-3759; Sato, et al., J. Invest. Dermatol. 2002, 118, 704-711; and Hanafusa, et al., J. Biol. Chem. 1999, 274, 27161-27167; which are each incorporated herein by reference for the purpose described herein). In certain embodiments, the nucleic acid encoded TGFβR inhibitor is optionally coupled with galunisertib. In certain embodiments, the inhibitor of a pro-inflammatory pathway is galunisertib.

In certain embodiments, provided compositions may comprise at least one nucleic acid comprising a sequence encoding a growth factor. Alternatively, in some embodiments, compositions comprising at least one nucleic acid may further comprise a growth factor.

Growth factors are a broad category of molecules that contribute to stimulating cell proliferation, wound healing, and occasionally cellular differentiation. Growth factors may include proteins and steroid hormones. Growth factors are involved in signaling between cells and play an important role in the immune system.

In certain embodiments, provided compositions comprise at least one nucleic acid encoding a growth factor, and/or at least one growth factor, wherein the nucleotide sequence encodes and/or the included growth factor is adrenomedullin, an angiopoietin (e.g., angiopoietin-1, angiopoietin-2), autocrine motility factor, a bone morphogenetic protein (BMP) (e.g., BMP1, BMP2, BMP3, BMP4, BMP5, BMP6, BMP7, BMP8a, BMP8b, BMP10, BMP11, or BMP15), a ciliary neurotrophic factor family protein (e.g., ciliary neurotrophic factor (CNTF), leukemia inhibitory factor (LIF), or interleukin-6 (IL-6)), a colony stimulating factor (e.g., macrophage colony-stimulating factor (M-CSF), granulocyte colony-stimulating factor (G-CSF), granulocyte macrophage colony-stimulating factor (GM-CSF), interleukin-3 (IL-3), or promegapoietin), epidermal growth factor (EGF), an ephrin ligand (e.g., ephrins A1, A2, A3, A4, A5, B1, B2, and B3), erythropoietin, a fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23), bovine growth hormone (BGH), a glial cell line-derived neurotrophic factor (GDNF) family ligand (e.g., GDNF, neurturin, artemin, or persephin), growth differentiation factor 9 (GDF9), hepatocyte growth factor (HGF), hepatoma-derived growth factor (HDGF), insulin, an insulin-like growth factor (IGF) (e.g., IGF-1 or IGF-2), an interleukin (IL) (e.g., IL-1, IL-2, IL-3, IL-4, IL-5, IL-6, or IL-7), keratinocyte growth factor (KGF), migration-stimulating factor (MSF), macrophage-stimulating protein (MSP), myostatin (GDF-8), a neuregulin (NRG) (e.g., NRG1, NRG2, NRG3, or NRG4), a neurotrophin (e.g., brain-derived neurotrophic factor (BDNF), nerve growth factor (NGF), neurotrophin-3 (NT-3), or neutrophin-4 (NT-4)), placental growth factor (PGF), platelet-derived growth factor (PDGF), renalase (RNLS), T-cell growth factor (TCGF), a transforming growth factor (TGF) (e.g., TGF-α and TGF-β1, TGF-β2, or TGF-β3), tumor necrosis factor-alpha (TNF-α), vascular endothelial growth factor (VEGF), or a WNT family protein (e.g., WNT1, WNT2, WNT3, WNT4, WNT5, WNT6, WNT7A, WNT7B, WNT8A, WNT8B, WNT9A, WNT9B, WNT10A, WNT10B, WNT11, or WNT16).

In certain embodiments, provided compositions may comprise at least one nucleic acid comprising a sequence encoding a hormone. Alternatively, in some embodiments, compositions comprising at least one nucleic acid may further comprise a hormone.

Hormones are a class of signaling molecules that can function in signaling between cells that are distant from each other with a multicellular organism. Hormones are a broad category of molecules that include peptide hormones, amino acid derivative hormones, steroid hormones, and eicosanoid hormones. Hormones play important immunomodulatory roles including regulating trafficking of lymphocytes, promoting differentiation of T cell populations, and modulating T cell and antigen-presenting cell activities, see e.g., Lähder et al., “Short but powerful: short peptide hormones and their role in autoimmune inflammation” J Neuroimmunol. (2009) 271 (1-2): 1-7, the contents of which are herein incorporated by reference for the purposes described herein. In certain embodiments, a hormone can be a naturally occurring hormone or a synthetic analog of a naturally occurring hormone.

In certain embodiments, provided compositions comprise at least one nucleic acid encoding a hormone, and/or at least one hormone, wherein the nucleotide sequence encodes and/or the included hormone is adrenocorticotropic hormone (ACTH), amylin, angiotensin, atrial natriuretic peptide (ANP), bradykinin, calcitonin, cholecystokinin (CCK), gastrin, ghrelin, glucagon, glucagon-like peptide-1 (GLP-1), glucagon-like peptide-2 (GLP-2), gonadotropin-releasing hormone (GnRH), growth hormone, follicle-stimulating hormone (FSH), insulin, kallidin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, or vasoactive intestinal peptide (VIP). In certain embodiments, provided compositions comprise at least one nucleic acid encoding a hormone, and/or at least one hormone, wherein the included hormone is and/or nucleotide sequence encodes abaloparatide, abarelix, afamelanotide, albiglutide, aviptadil, buserelin, carbetocin, cargutocin, carpertide, cetrorelix, degarelix, deslorelin, desmopressin, dulaglutide, elcatonin, exenatide, felypressin, ganirelix, ghrelin, gonadorelin, goserelin, histrelin, icatibant, lanreotide, leuprorelin, liraglutide, lixisenatide, nafarelin, nesiritide, octreotide, ornipressin, pasireotide, pramlintide, saralasin, taltirelin, teduglutide, teriparatide, terlipressin, tesamorelin, tetracosactide, thymopentin, or triptorelin.

In certain embodiments, provided compositions comprising at least one nucleic acid encoding a hormone, and/or at least one hormone are useful in treating cancer. In certain embodiments, provided compositions comprising at least one nucleic acid encoding a hormone, and/or at least one hormone are useful in treating a non-proliferative disease. In certain embodiments, provided compositions comprising at least one nucleic acid encoding a hormone, and/or at least one hormone are useful in treating acromegaly, acute decompensated heart failure, bleeding esophageal varices, breast cancer, Cushing's disease, diabetes insipidus, endometriosis, erectile dysfunction, erythropoietic protoporphyria, hereditary angioedema, HIV lipodystrophy, hypercalcemia, hypoglycemia, neuroendocrine tumors, nocturia, osteoporosis, Paget's disease, postpartum hemorrhage, precocious puberty, primary amenorrhea, prostate cancer, renovascular hypertension, short bowel syndrome, spinocerebellar degeneration, type 1 diabetes, type 2 diabetes, various autoimmune diseases, various infectious diseases, various inflammatory diseases, vasodilatory shock, or West syndrome; see e.g., Lau and Dunn. “Therapeutic peptides: historical perspectives, current development trends, and future directions” Bioorg Med Chem (2018) 26 (10): 2700-2707, the contents of which are herein incorporated by reference for the purposes described herein.

In certain embodiments, provided compositions may comprise a mono-therapeutic nucleic acid encoding a single immune modulator. In certain embodiments, provided compositions may comprise multiple nucleic acids encoding multiple different immune modulators. In certain embodiments, provided compositions may comprise at least one nucleic acid which encodes multiple different immune modulators functionally linked on the same nucleic acid strand. In certain embodiments, multiple nucleic acids may each encode a portion or part of a final protein or RNA product, wherein the final product is created as a result of homologous recombination, splicing, and/or tertiary or quaternary amino acid sequences.

### Polynucleotide Agent Carrier(s)

In certain embodiments, provided compositions may comprise at least one polymeric biomaterial, at least one nucleic acid, and optionally one or more polynucleotide agent carriers. In some embodiments, provided compositions may comprise at least one nucleic acid and one or more polynucleotide agent carriers. In some embodiments, at least one nucleic acid of the provided composition is complexed to a polynucleotide agent carrier. In some embodiments, provided nucleic acids are loaded in the polynucleotide agent carrier. In some embodiments, the provided composition comprises at least one polynucleotide agent carrier that may be: lipids, lipoplexes, liposomes, lipid nanoparticles (LNPs), stable nucleic acid lipid particles (SNALPs), ionizable lipid-based SNALPs, lipidoids, DOTAP, DOTMA, DOPE, DSPE, DODMA, DODAP, DOGS, DC-Chol, PC, Chol, cationic polymers, amphiphilic copolymer(s), PEG, polyamino acid, polylactice or glycolic acid, polycaprolactone, poly(beta-amino esters), protamine, polyethyleneimine, gold, silver, as well as combinations and/or derivatives thereof; see e.g., Ni et al. Life (Basel) 2019; 9 (3): 59, the contents of which are herein incorporated in their entirety for the purposes described herein. In certain embodiments, provided compositions do not contain one or more polynucleotide agent carriers.

In some embodiments, nucleic acids of exemplary compositions may function without being endocytosed. In some embodiments, provided nucleic acids may function following endocytosis. In some embodiments, provided nucleic acids of the exemplary compositions do not require a polynucleotide agent carrier for endocytosis. In some embodiments, the endocytosis of nucleic acids of the exemplary composition is aided by polynucleotide agent carriers. In embodiments where the nucleic acid of the exemplary drug delivery composition is endocytosed, the endocytosing cell can be said to be transfected. Transfection can occur with or without the aid of a nucleic acid carrier.

Polynucleotides are generally macromolecular, and may be highly hydrophilic/anionic while displaying relatively complex supramolecular assemblies. To successfully deliver intact polynucleotides to cells, significant intracellular and extracellular barriers must be overcome. Initially, polynucleotides must be protected from biodegradation by extracellular (e.g., serum and tissue-specific) nucleases. Once nucleic acids reach the cell, if they encode an active agent, they must permeate through the anionic plasma membrane bilayer, a dynamic structure composed of phospholipids, membrane proteins, and cholesterol held together primarily through hydrophobic interactions. The desired result of transfection may then only be observed if the nucleic acid escapes intracellular degradation and carries out its function (e.g., activating the innate immune system either directly, or through the creation of an encoded product). A method which delivers the polynucleotide to all target cells while causing minimal or negligible toxicity is desired. One skilled in the art will recognize that there are numerous approaches developed for transfection, but that there is no universally effective material and/or method that functions optimally in all cells.

In certain embodiments, to aid with endocytosis and/or transfection, the nucleic acid of provided compositions may be modified through various base, backbone, or sugar modifications, which can act to facilitate nucleic acid stability and transfection efficiency. In certain embodiments, chemical modifications may be employed to one, two, or all three nucleic acid components, sugar-phosphate backbone, sugar moiety and heterocyclic base moiety. In certain embodiments, the nucleic acid of the exemplary composition may encompass any, all, or none of the following alterations. Sugar-phosphate backbone modifications include but are not limited to: Phosphorothioate (PS), Boranophosphate (BP), Phosphonoacetate (PACE), Morpholino (PMO), and Peptide Nucleic Acid (PNA). Sugar moiety modifications include but are not limited to: 2′-O-methyl (2′-O-ME), 2′-O-methoxyethyl(2′-MOE), 2′-Fluoro (2′-F), 2′-deoxy-2′-fluoro-β-d-arabinonucleic acid (2′-FANA), 4′-thio(4′-S), 4′-C-aminomethyl-2′-O-Me, locked nucleic acid (LNA), Tricyclo DNA (tcDNA), unlocked nucleic acid (UNA). Heterocyclic base modifications include but are not limited to: 5-bromo-uracil, 5-iodo-uracil, 2,6-diamino-purine, and 5-methyl cytosine.

In certain embodiments, provided compositions may comprise at least one nucleic acid, and at least one polynucleotide chemical carrier. Chemical carriers are often rationally designed to prevail over extracellular and intracellular obstacles that prevent nucleic acid entry into cells. Polynucleotide carriers may leverage the anionic charge of nucleic acids to either electrostatically condense them to a relatively smaller size suitable for internalization, or encapsulate them behind a physical barrier, and/or in some cases adsorb them onto surfaces. These effects may mask the negative charge on nucleic acids. In sum, these effects may to protect the nucleic acids from endonucleases, facilitate internationalization, and perhaps facilitate transcription and/or translation.

In certain embodiments, provided compositions may comprise at least one nucleic acid, and at least one cell penetrating peptide (CPP). CPPs are usually relatively short (5-30 amino acid long) peptides of diverse characteristics capable of carrying various cargos, including polynucleotides, across cellular membranes. CPPs can be classified into cationic peptides rich in basic amino acids such as lysine and arginine, and amphipathic peptides, which contain both hydrophilic and hydrophobic amino acids. Amphipathic peptides can acquire different secondary structures depending on the spatial arrangement of hydrophobic and hydrophilic residues under different ambient conditions. One skilled in the art will recognize that the charge number and density, size, hydrogen bonds, and secondary structure of CPPs may play a critical role in determining their interaction with different components of the cell membrane, and hence their internalization. As carriers, CPPs can be either directly conjugated to polynucleotides or ionically complexed with the nucleic acids. The covalent conjugation approach allows for the generation of conjugates with a well-defined structure and stoichiometry. To further improve their in vivo stability, CPPs can be modified with fatty acids or cholesterol. Examples of CPPs which may be comprised in provided compositions include but are not limited to: oligoarginine (R9), penetratin, lactoferrin, PepFect 6 peptide, pepR, pepM, arginine, cysteine, gelatin, and HIV-1 protein Tat-derived peptides.

In certain embodiments, provided compositions may include hydrophobes or lipids. Cationic hydrophobic molecules are commonly used vehicles for nucleic acid delivery as they can be relatively easily synthesized and chemically modified for different applications. In certain embodiments, the polynucleotide agent carrier comprised in exemplary compositions may comprise, but is not limited to, commercially available cationic lipids: N-[1-(2,3-dioleoyloxy)-propel]-N,N,N-trimethylammonium (DOTMA, Lipofectin); 2,3-dioleyloxy-N-[2-spermine carboxamide] ethyl-N,N-dimethyl-1-propanammonium trifluoroacetate (DOSPA); 1,2-dioleoyl-3-trimethylammonium-propane (DOTAP); N-[1-(2,3-dimyristyloxy) propyl]-N,N-dimethyl-N-(2-hydroxyethyl) ammonium bromide (DMRIE); 3b-[N-(N,N-dimethylaminoethane) carbamoyl] cholesterol (DC-Chol); dioctadecyl amidoglyceryl spermine (DOGS, Transfectam); and/or dimethyldioctadecylammonium bromide (DDAB). Cationic lipids can be formulated with nucleic acids as stable nucleic acid lipid particles (SNALPs).

In certain embodiments, provided compositions may comprise polymeric carriers of nucleic acids. In certain embodiments, polymeric carriers may be chemically conjugated or complexed via electrostatic interactions or enveloped around nucleic acids to facilitate their delivery to target cells. In some embodiments, polymeric carriers of nucleic acid may comprise but are not limited to: polyethyleneimine (PEI), chitosan, PEG-PPL=PLLeu, PEI-CG-PEI, PgP, and/or any combination thereof.

### Modulators of the Innate Immune Response

In certain embodiments, provided compositions comprise at least one nucleic acid acting as or encoding a modulator of the innate immune response. In certain embodiments, provided compositions may comprise more than one nucleic acid acting as or encoding modulators of the innate immune response. The major functions of the innate immune response include recruiting immune cells to sites of infection through the production of chemical factors, including specialized chemical mediators (e.g., cytokines); activation of the complement cascade to identify bacteria, activate cells, and promote clearance of antibody complexes or dead cells; identification and removal of foreign substances present in organs, tissues, blood, and lymph by specialized white blood cells; activation of the adaptive immune system through a process known as antigen presentation; and acting as a physical and chemical barrier to infectious agents (e.g., epithelial surfaces, gastrointestinal tract). Typically, leukocytes are the white blood cells that carry out the actions of the innate immune system. These cells include natural killer cells, mast cells, eosinophils, basophils, macrophages, neutrophils, and dendritic cells. These cells function within the immune system by identifying and eliminating pathogens that might cause infection.

### Inhibitors of the Innate Immune Response

The present disclosure recognizes, among other things, that inhibiting the inflammatory response may reduce the risk of cancer recurrence and thus prolong survival. The present disclosure recognizes, among other things, that inhibiting pro-inflammatory immune response mediated by p38 mitogen-activated protein kinase (MAPK) pathway (e.g., by administration of a nucleic acid acting as or encoding a p38 MAPK inhibitor) at a target site (e.g., a tumor resection site) can reduce the risk of cancer recurrence and thus prolong survival. It is unexpected that inhibition of MAPK can promote antitumor immunity since MAPK-targeted therapy (e.g., inhibition of the BRAF/MEK/ERK module) was reported to induce transcriptional signatures associated with resistance to anti-PD-1 immune checkpoint blockade therapy, which may in turn negatively impact responsiveness to anti-PD-1/L1 cancer therapy (See, e.g., Hugo, et al., Cell 2016, 165, 35-44; which is incorporated herein by reference for the purpose described herein).

Accordingly, in some embodiments, provided herein are compositions comprising at least one nucleic acid acting as or encoding an inhibitor of proinflammatory immune response mediated by p38 mitogen-activated protein kinase (MAPK) pathway. In some embodiments, compositions provided herein may comprise more than one nucleic acids acting as or encoding inhibitors of a proinflammatory immune response mediated by p38 mitogen-activated protein kinase (MAPK) pathway. In some embodiments, and where applicable, the inhibitors of the pro proinflammatory immune response mediated by p38 mitogen-activated protein kinase (MAPK) pathway may be delivered as nucleic acids which encode the specific inhibitor.

The p38 family of MAPKs includes the p38α, p38β, p38γ, and p38δ isoforms. p38 MAPK is activated by a large number of immune receptors, thus inhibition of a signaling module or a regulatory target that functions either upstream or downstream of p38 may provide an efficacious and selective method of inhibiting the molecular pathway and the proinflammatory immune response it mediates.

For example, p38 MAPK may be activated by mitogen-activated protein kinase kinase 3 (MAP2K3), mitogen-activated protein kinase kinase 6 (MAP2K6), mitogen-activated protein kinase kinase kinase 1 (MAP3K1), and/or mitogen-activated protein kinase kinase kinase 4 (MAP3K4). Thus, inhibiting upstream targets of p38 MAPK may be effective in inhibiting the p38 MAPK pathway.

Inhibition of downstream targets of p38 MAPK may also be an effective means of inhibiting the p38 MAPK pathway. Downstream of p38 MAPK, for example, mitogen-activated protein kinase interacting protein kinases 1 and 2 (MNK1 and MNK2) are activated by the p38 MAPK pathway. The MNK kinases play important roles in regulating mRNA translation and, as a result, are key mediators of oncogenic progression, drug resistance, production of pro-inflammatory cytokines and cytokine signaling. Mitogen- and stress-activated kinase 1 and 2 (MSK1 and MSK2) are also downstream targets of p38 MAPK, and affect inflammatory responses. MAP kinase-activated protein kinase 2, 3, and 5 (MK2, MK3, MK5) are activated by p38 MAPK and are involved in cellular stress and inflammatory responses.

In view of the foregoing, inhibition of the p38 MAPK pathway through at least one nucleic acid acting as or encoding an inhibitor of upstream or downstream components of the p38 MAPK pathway may provide a therapeutic strategy for the treatment of cancer. In particular, local inflammatory wound response and systemic inflammation processes together may activate dormant micrometastases or induce the propagation of residual cancer cells, thus increasing the risk of cancer recurrence. Therefore, inhibiting the pro-inflammatory immune response mediated by the p38 MAPK pathway at a tumor resection site can reduce the risk of cancer recurrence and prolong survival of a subject.

In certain embodiments, the inhibitor of proinflammatory immune response mediated by the p38 MAPK pathway is a p38 MAP kinase inhibitor. In certain embodiments, provided compositions may comprise at least one nucleic acid acting as or encoding an inhibitor of p38α, p38β, p38γ, and/or p38δ MAP kinase. In certain embodiments, provided compositions may comprise at least one nucleic acid acting as or encoding a p38 MAP kinase inhibitor, and may further comprise semapimod, pexmetinib, BMS-582949, losmapimod, pamapimod, ralimetinib, doramapimod, VX-702, VX-745, TAK-715, SB239063, SB202190, SB203580, SCIO 469, PH-797804, AZD7624, ARRY-797, ARRY-614, AVE-9940, LY3007113, skepinone-L, UM-164, SCIO 323, SX-011, SK-F860002, SB706504, SB681323, CHF-6297, RWJ-67657, Org48762-0, ML3403, JX-401, EO-1428, DBM 1285, AMG-548, AL-8697, PD-169316, PF-03715455, PH-797804, selonsertib, sorafenib, dilmapimod and/or any combination thereof. In certain embodiments, provided compositions may comprise at least one nucleic acid acting as or encoding a p38 MAP kinase inhibitor, and may further comprise quinazolinone, pyrimido-pyrimidone, pyrido-pyrimidone, pyrazole, quinolinone, naphthyridinone core structure, and/or any combination thereof. In certain embodiments, provided compositions may further comprise the p38 MAP kinase inhibitor losmapimod.

In certain embodiments, the p38 MAP kinase inhibitor binds to the ATP binding site of the p38 MAP kinase. In certain embodiments, the p38 MAP kinase inhibitor is an allosteric inhibitor of p38 MAP kinase.

In certain embodiments, provided compositions may comprise at least one nucleic acid acting as or encoding an inhibitor of proinflammatory immune response mediated by the p38 MAPK pathway, wherein the inhibitor acts upon an upstream effector of p38 MAPK. In certain embodiments, the at least one nucleic acid acting as or encoding an inhibitor of proinflammatory immune response mediated by the p38 MAPK pathway is an inhibitor of RIPK1, RIPK2, RIPK3, RIPK4, RAC1, CDC42, MTK1, TAK1, MEKK1, MEKK2, MEKK3, MEKK4, DLK, MLK2, TAO1, TAO2, TLP2, TPL2, ASK1, MKK3, MKK4, and/or MKK6. In certain embodiments, the at least one nucleic acid acting as or encoding an inhibitor of proinflammatory immune response mediated by the p38 MAPK pathway is an inhibitor of a downstream effector of p38 MAPK. In certain embodiments, the at least one nucleic acid acting as or encoding an inhibitor of proinflammatory immune response mediated by the p38 MAPK pathway is an inhibitor of MK2, MK3, MNK1, MNK2, MSK1, MSK2, MSK3, RSK, PP2A, and/or cPLA2.

### Activators of the Innate Immune Response

In some embodiments, provided compositions may comprise an activator of the innate immune response. In some embodiments, provided compositions may comprise more than one activator of innate immune response. In some embodiments, where applicable, activators of the innate immune response may be delivered as nucleic acids which act as or encode the specific activator. The major functions of the innate immune response include recruiting immune cells to sites of infection through the production of chemical factors, including specialized chemical mediators (e.g., cytokines); activation of the complement cascade to identify bacteria, activate cells, and promote clearance of antibody complexes or dead cells; identification and removal of foreign substances present in organs, tissues, blood, and lymph by specialized white blood cells; activation of the adaptive immune system through a process known as antigen presentation; and acting as a physical and chemical barrier to infectious agents (e.g., epithelial surfaces, gastrointestinal tract). Typically, leukocytes are the white blood cells that carry out the actions of the innate immune system. These cells include natural killer cells, mast cells, eosinophils, basophils, macrophages, neutrophils, and dendritic cells. These cells function within the immune system by identifying and eliminating pathogens that might cause infection.

In certain embodiments, provided compositions may further comprise activation of the proinflammatory innate immune response mediated by p38 mitogen-activated protein.

In certain embodiments, provided compositions may further comprise at least one activator of innate immune response.

In certain embodiments, provided compositions comprise at least one nucleic acid acting as or encoding an activator of the innate immune response is a ligand of a pattern recognition receptor (PRR). In certain embodiments, the at least one nucleic acid acting as or encoding an activator of innate immune response is an agonist of a pattern recognition receptor (PRR).

In certain embodiments, provided compositions comprise at least one nucleic acid acting as or encoding an activator of innate immune response, wherein the activator is an inducer of type I interferon. In certain embodiments, the activator of innate immune response is a recombinant interferon.

In certain embodiments, provided compositions comprise at least one nucleic acid acting as or encoding an activator of innate immune response, wherein the activator is an effective inducer of activation and/or proliferation of NK cells. In certain embodiments, “effective inducer” refers to an activator of innate immune response that directly induces activation and/or proliferation of NK cells.

In certain embodiments, provided compositions comprise at least one nucleic acid acting as or encoding an activator of innate immune response, wherein the activator is an effective inducer of activation and/or maturation of dendritic cells. In certain embodiments, “effective inducer” refers to an activator of innate immune response that directly induces activation and/or maturation of dendritic cells.

In certain embodiments, provided compositions comprise at least one nucleic acid acting as or encoding an activator of innate immune response, wherein the activator is an effective inducer of type I interferon by dendritic cells. In certain embodiments, “effective inducer” refers to an activator of innate immune response that directly induces type I interferon by dendritic cells.

In certain embodiments, provided compositions comprise at least one nucleic acid which acts as or encodes an activator of innate immunity. In some embodiments, provided compositions comprise at least one nucleic acid which acts as or encodes an activator of innate immunity, and an optional additional activator of innate immune response, wherein the additional activator is a small molecule or a biologic. In certain embodiments, the activator of innate immune response is a small molecule. In certain embodiments, the activator of innate immune response is a biologic.

In certain embodiments, an activator of innate immune response is a stimulator of interferon genes (STING) agonist, a cytosolic DNA sensor (CDS) agonist, a Toll-like receptor (TLR) agonist, a C-type lectin receptor (CLR) agonist, a NOD-like receptor (NLR) agonist, a RIG-I-like receptor (RLR) agonist, and/or an inflammasome inducer.

In certain embodiments, an activator of innate immune response is a stimulator of interferon genes (STING) agonist, a Toll-like receptor (TLR) agonist, and/or a NOD-like receptor (NLR) agonist. In certain embodiments, an activator of innate immune response is a stimulator of interferon genes (STING) agonist and/or a Toll-like receptor (TLR) agonist. In certain embodiments, an activator of innate immune response is a stimulator of interferon genes (STING) agonist, a TLR7 agonist, and/or a TLR8 agonist.

In certain embodiments, an activator of innate immune response is 3′3′-cGAMP, 2′3′-cGAMP, 2′3′-cGAM (PS) 2 (Rp/Rp), 2′3′-cGAM (PS) 2 (Rp/Sp), 2′2′-cGAMP, c-di-AMP, 2′3′-c-di-AMP, 2′3′-c-di-AMP (PS) 2 (Rp/Rp), 2′3′-c-di-AMP (PS) 2 (Rp/Sp), c-di-GMP, c-di-IMP, HSV-60, ISD, VACV-70, poly(dA: dT), poly(dG: dC), heat-killed bacteria, lipoglycans, lipopolysaccharides (LPS), lipoteichoic acids, peptidoglycans (PGNs), synthetic lipoproteins, poly(A: U), poly(I: C), Monophosphoryl Lipid A (MPLA), GSK1795091, G100, SD-101, MGN1703, CMP-001, flagellin (FLA), polyU, poly(dT), gardiquimod, imiquimod (R837), base analogs, adenine analogs, guanosin analogs, purine derivatives, benoazepine analogs, imidazoquinolines, thiazoquinolines, loxoribine, resiquimod (R848), dactolisib, sumanirole, N1-glycinyl [4-((6-amino-2-(butylamino)-8-hydroxy-9H-purin-9-yl)methyl)benzoyl] spermine (CL307), CL264, CL097, CL075, CL347, CL401, CL413, CL419, CL531, CL553, CL572, MEDI9197, MEDI5083, hypoxanthine, TL8-506, PF-4878691, isatoribine, SM-324405, SM-324406, AZ12441970, AZ12443988, CpG oligonucleotides, bacterial DNA, beta glucans, beta glucans from fungal and bacterial cell walls, γ-D-Glu-mDAP (iE-DAP), iE-DAP derivatives, muramyl dipeptide (MDP), MDP derivatives, 5′ triphosphate double stranded RNA, poly(dA: dT), ATP, chitosan, aluminum potassium sulfate, calcium pyrophosphate dehydrate, silica dioxide, MurNAc-L-Ala-γ-D-Glu-mDAP (M-TriDAP), a xanthenone analog (e.g., DMXAA; vadimezan), a TREX1 inhibitor, a cyclic dinucleotide, LHC165, GSK-2245035, RG7854, GS-9620, GS-9688, EMD1201081, PF-3512676, BO-112, RGT-100, MK-1454, SB-11285, NKTR-262, CDX-301, 2′3′-c-di-GMP, cAIMP, cAIM (PS) 2 (Rp/Sp), derivatives thereof, and/or pharmaceutically acceptable salts thereof.

In certain embodiments, an activator of innate immune response is a fluorinated derivative of any of the innate immune response activators described herein. In certain embodiments, an activator of innate immune response is difluoroinated cAIMP (c-(2′FdAMP-2′FdIMP)). In certain embodiments, an activator of innate immune response is difluoroinated cAIM (PS) 2 (Rp/Sp). In certain embodiments, an activator of innate immune response is an O-methylated derivative of any of the above activators.

In certain embodiments, an activator of innate immune response is 3′3′-cGAMP, 2′3′-cGAMP, 2′3′-cGAM (PS) 2 (Rp,Rp), 2′3′-cGAM (PS) 2 (Rp,Sp), 2′2′-cGAMP, c-di-AMP, 2′3′-c-di-AMP, 2′3′-c-di-AM (PS) 2 (Rp,Rp), 2′3′-c-di-AM (PS) 2 (Rp,Sp), c-di-GMP, 2′3′-c-di-GMP, 2′3′-c-di-GM (PS) 2 (Rp,Rp), 2′3′-c-di-GM (PS) 2 (Rp,Sp), c-di-IMP, resiquimod, CpG oligonucleotides, polyinosinic: polycytidylic acid, LHC165, GSK-2245035, RG7854, GS-9620, GS-9688, EMD1201081, PF-3512676, BO-112, RGT-100, MK-1454, SB-11285, NKTR-262, CDX-301, 2′3′-c-di-GMP, cAIMP, cAIM (PS) 2 (Rp/Sp), and/or pharmaceutically acceptable salts thereof.

In certain embodiments, an activator of innate immune response is a fluorinated derivative of 3′3′-cGAMP, 2′3′-cGAMP, 2′3′-cGAM (PS) 2 (Rp,Rp), 2′3′-cGAM (PS) 2 (Rp,Sp), 2′2′-cGAMP, c-di-AMP, 2′3′-c-di-AMP, 2′3′-c-di-AM (PS) 2 (Rp,Rp), 2′3′-c-di-AM (PS) 2 (Rp,Sp), c-di-GMP, 2′3′-c-di-GMP, 2′3′-c-di-GM (PS) 2 (Rp,Rp), 2′3′-c-di-GM (PS) 2 (Rp,Sp), c-di-IMP, 2′3′-c-di-GMP, cAIMP, cAIM (PS) 2 (Rp/Sp), and/or pharmaceutically acceptable salts thereof.

In certain embodiments, an activator of innate immune response is an O-methylated derivative of 3′3′-cGAMP, 2′3′-cGAMP, 2′3′-cGAM (PS) 2 (Rp,Rp), 2′3′-cGAM (PS) 2 (Rp,Sp), 2′2′-cGAMP, c-di-AMP, 2′3′-c-di-AMP, 2′3′-c-di-AM (PS) 2 (Rp,Rp), 2′3′-c-di-AM (PS) 2 (Rp,Sp), c-di-GMP, 2′3′-c-di-GMP, 2′3′-c-di-GM (PS) 2 (Rp,Rp), 2′3′-c-di-GM (PS) 2 (Rp,Sp), c-di-IMP, and/or pharmaceutically acceptable salts thereof.

In certain embodiments, an activator of innate immune response is 2′3′-cGAMP, 2′3′-c-di-AM (PS) 2 (Rp,Rp), MurNAc-L-Ala-γ-D-Glu-mDAP (M-TriDAP), c-di-GMP, and/or resiquimod. In certain embodiments, an activator of innate immune response is 2′3′-cGAMP, 2′3′-c-di-AM (PS) 2 (Rp,Rp), MurNAc-L-Ala-γ-D-Glu-mDAP (M-TriDAP), and/or resiquimod. In certain embodiments, an activator of innate immune response is 2′3′-cGAMP, 2′3′-c-di-AM (PS) 2 (Rp,Rp), and/or resiquimod. In certain embodiments, an activator of innate immune response is 2′3′-c-di-AM (PS) 2 (Rp,Rp) and/or resiquimod. In certain embodiments, an activator of innate immune response is cAIMP and/or its fluorinated derivative. In certain embodiments, an activator of innate immune response is difluorinated cAIMP.

In certain embodiments, an activator of innate immune response is 2′3′-cGAMP, or a pharmaceutically acceptable salt thereof. In particular, 2′3′-cGAMP (cyclic [G (2′,5′) pA (3′,5′) p]) has been described to function as an endogenous second messenger, inducing STING-dependent type I interferon response. 2′3′-cGAMP has also been shown to be an effective adjuvant that boosts the production of antigen-specific antibodies and T cell responses in mice. 2′3′-cGAMP exercises antiviral functions in the cell where it is produced but can also cross cell membranes by passive diffusion to exert effects on neighboring cells.

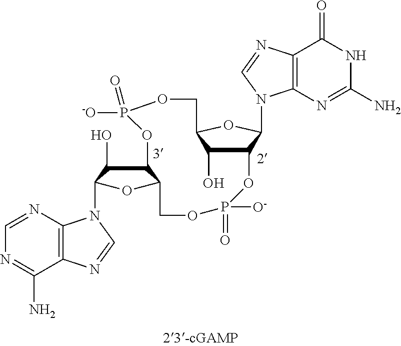

In certain embodiments, an activator of innate immune response is 2′3′-c-di-AM (PS) 2 (Rp,Rp), or a pharmaceutically acceptable salt thereof. 2′3′-c-di-AM (PS) 2 (Rp,Rp) is the Rp,Rp-isomer of the 2′3′ bisphosphorothioate analog of 3′3′-cyclic adenosine monophosphate (c-di-AMP). It is also a STING agonist.

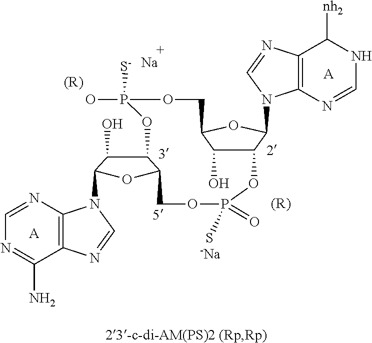

In certain embodiments, an activator of innate immune response is cAIMP, its difluorinated derivative, its difluorinated bisphosphorothiate derivate (cAIM (PS) 2 (Rp/Sp)), and/or a pharmaceutically acceptable salt thereof. cAIMP and its derivatives are also STING agonists.

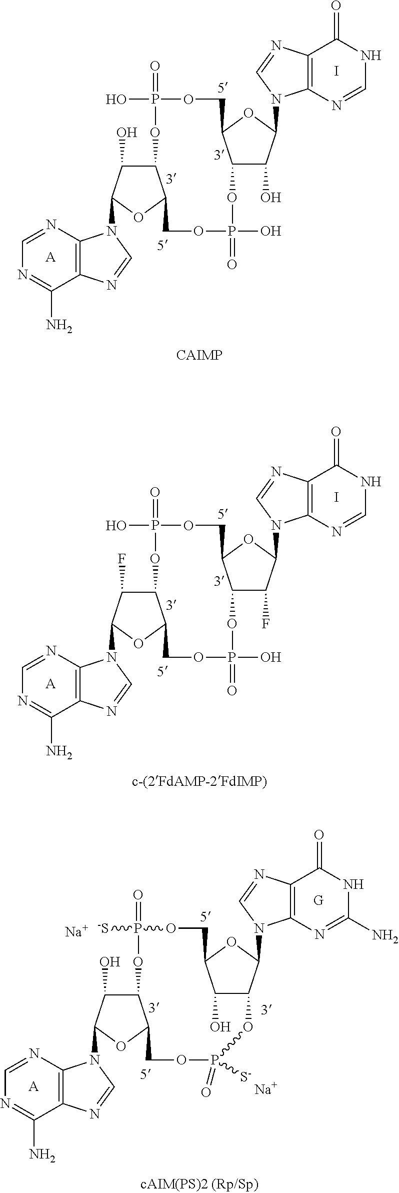

In certain embodiments, an activator of innate immune response is a STING agonist, wherein the STING agonist is a cyclic dinucleotide. In certain embodiments, the cyclic dinucleotide is any cyclic dinucleotide disclosed in U.S. Pat. No. 10,106,574, granted Oct. 23, 2018, the entire contents of which are incorporated herein by reference. In certain embodiments, the cyclic dinucleotide is any cyclic dinucleotide disclosed in U.S. Pat. No. 10,131,686, granted Nov. 20, 2018, the entire contents of which are incorporated herein by reference.

In certain embodiments, an activator of innate immune response is MK-1454.

In certain embodiments, an activator of innate immune response is a cytosolic DNA sensor (CDS) agonist. In certain embodiments, the CDS agonist is a cyclic GMP-AMP synthase (cGAS) agonist.

In certain embodiments, an activator of innate immune response is any STING agonist or cGAS agonist disclosed in U.S. Pat. No. 10,336,786 granted Jul. 2, 2019, the entire contents of which are incorporated herein by reference. In certain embodiments, the activator of innate immune response is any STING agonist or cGAS agonist disclosed in U.S. patent application U.S. Ser. No. 14/268,967, filed May 2, 2014, the entire contents of which are incorporated herein by reference. In certain embodiments, the activator of innate immune response is any STING agonist or cGAS agonist disclosed in U.S. Pat. No. 9,840,533, granted Dec. 12, 2017, the entire contents of which are incorporated herein by reference. In certain embodiments, the activator of innate immune response is any STING agonist or cGAS agonist disclosed in U.S. Pat. No. 10,176,292, granted Jan. 8, 2019, the entire contents of which are incorporated herein by reference.

In certain embodiments, an activator of innate immune response is any STING agonist disclosed in U.S. patent application U.S. Ser. No. 13/057,662, filed Jun. 14, 2011, the entire contents of which are incorporated herein by reference. In certain embodiments, an activator of innate immune response is any STING agonist disclosed in U.S. Pat. No. 9,695,212, granted Jul. 4, 2017, the entire contents of which are incorporated herein by reference. In certain embodiments, an activator of innate immune response is any STING agonist disclosed in U.S. patent application U.S. Ser. No. 15/035,432, filed May 19, 2016, the entire contents of which are incorporated herein by reference. In certain embodiments, an activator of innate immune response is any STING agonist disclosed in U.S. patent application U.S. Ser. No. 16/069,201, filed Jan. 11, 2018, the entire contents of which are incorporated herein by reference. In certain embodiments, an activator of innate immune response is any STING agonist disclosed in U.S. Pat. No. 10,604,542, granted Mar. 31, 2020, the entire contents of which are incorporated herein by reference. In certain embodiments, an activator of innate immune response is any STING agonist disclosed in International Patent Application PCT/US2014/038525, filed May 18, 2014, the entire contents of which are incorporated herein by reference. In certain embodiments, an activator of innate immune response is any STING agonist disclosed in U.S. Pat. No. 9,770,467, granted Sep. 26, 2017, the entire contents of which are incorporated herein by reference. In certain embodiments, an activator of innate immune response is any STING agonist disclosed in International Patent Application PCT/IB2016/057265, filed Jan. 12, 2016, the entire contents of which are incorporated herein by reference.

In certain embodiments, an activator of innate immune response is MurNAc-L-Ala-γ-D-Glu-mDAP (M-TriDAP), or a pharmaceutically acceptable salt thereof. M-TriDAP is a peptidoglycan (PGN) degradation product found mostly in Gram-negative bacteria. M-TriDAP is recognized by the intracellular sensor NOD1 (CARD4) and to a lesser extent NOD2 (CARD15). Recognition of M-TriDAP by NOD1/NOD2 induces a signaling cascade involving the serine/threonine RIP2 (RICK, CARDIAK) kinase, which interacts with IKK leading to the activation of NF-κB and the production of inflammatory cytokines such as TNF-α and IL-6. M-TriDAP induces the activation of NF-κB at similar levels to Tri-DAP.

In certain embodiments, an activator of innate immune response is a TLR7 agonist. In certain embodiments, the activator of innate immune response is a TLR8 agonist. In certain embodiments, the activator of innate immune response is a TLR7 agonist and a TLR8 agonist.

In certain embodiments, an activator of innate immune response is an immune response modifier (IRM).

In certain embodiments, an activator of innate immune response is any IRM disclosed in U.S. Pat. No. 5,714,608, granted Feb. 3, 1998, the entire contents of which are incorporated herein by reference. In certain embodiments, an activator of innate immune response is any IRM disclosed in U.S. Pat. No. 6,039,969, granted Mar. 21, 2000, the entire contents of which are incorporated herein by reference. In certain embodiments, an activator of innate immune response is any IRM disclosed in U.S. Pat. No. 6,200,592, granted Mar. 13, 2000, the entire contents of which are incorporated herein by reference. In certain embodiments, an activator of innate immune response is any IRM disclosed in U.S. Pat. No. 4,689,338, granted Aug. 25, 1987, the entire contents of which are incorporated herein by reference. In certain embodiments, an activator of innate immune response is any IRM disclosed in U.S. Pat. No. 5,446,153, granted Aug. 29, 1995, the entire contents of which are incorporated herein by reference. In certain embodiments, an activator of innate immune response is any IRM disclosed in U.S. Pat. No. 6,194,425, granted Feb. 2, 2001, the entire contents of which are incorporated herein by reference. In certain embodiments, an activator of innate immune response is any IRM disclosed in U.S. Pat. No. 6,110,929 granted Aug. 29, 2000, the entire contents of which are incorporated herein by reference. In certain embodiments, an activator of innate immune response is any IRM disclosed in U.S. Pat. No. 7,544,697, granted Jun. 9, 2009, the entire contents of which are incorporated herein by reference.

In certain embodiments, an activator of innate immune response is CL307 (N1-glycinyl [4-((6-amino-2-(butylamino)-8-hydroxy-9H-purin-9-yl)methyl)benzoyl] spermine), or a pharmaceutically acceptable salt thereof. CL307 is a very potent TLR7 agonist. Titration experiments have showed that CL307 induces robust NF-κB activation even at concentrations as low as 20 nM (10 ng/ml).

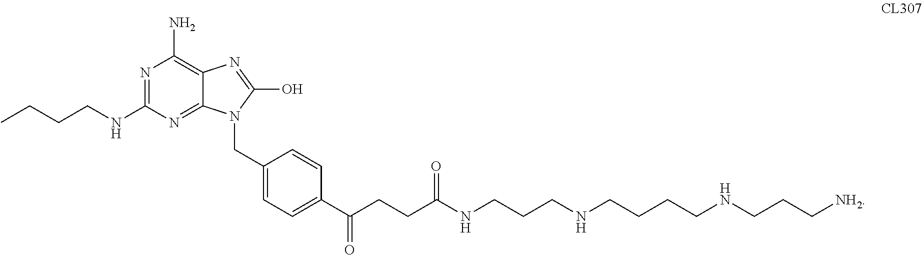 [SMILES: CCCCNc1nc(N)c2nc(O)n(Cc3ccc(C(=O)CCC(=O)NCCCNCCCCNCCCN)cc3)c2n1]

In certain embodiments, an activator of innate immune response is CL264, or a pharmaceutically acceptable salt thereof. CL264 induces the activation of NF-κB and the secretion of IFN-α in TLR7-expressing cells. CL264 is a TLR7-specific ligand, it does not stimulate TLR8 even at high concentrations (>10 μg/ml). In TLR7-transfected HEK293 cells, CL264 triggers NF-κB activation at a concentration of 0.1 μM which is 5-10 times less than imiquimod.

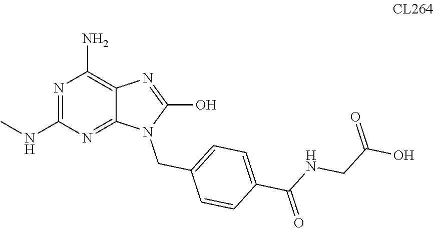 [SMILES: CNc1nc(N)c2nc(O)n(Cc3ccc(C(=O)NCC(=O)O)cc3)c2n1]

In certain embodiments, an activator of innate immune response is loxoribine, or a pharmaceutically acceptable salt thereof. Loxoribine is a guanosine analog derivatized at positions N⁷ and C⁸. This nucleoside is a very powerful stimulator of the immune system. Loxoribine activates the innate immune system through TLR7 and this activation requires endosomal maturation. Loxoribine recognition is restricted to TLR7.

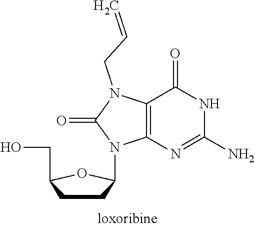

In certain embodiments, an activator of innate immune response is hypoxanthine, or a pharmaceutically acceptable salt thereof. Hypoxanthine is a naturally occurring purine derivative.

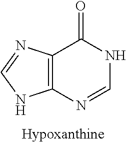

In certain embodiments, an activator of innate immune response is TL8-506, or a pharmaceutically acceptable salt thereof. TL8-506 is a benzoazepine compound, an analog of the Toll-like receptor 8 (TLR8) agonist VTX-2337. TL8-506 activates TLR8 more potently than R848 and CL075. TL8-506 is ˜50× and ˜25× more potent in inducing NF-κB activation in TLR8-transfected HEK293 cells than R848 and CL075, respectively. TL8-506 is a selective agonist of TLR8.

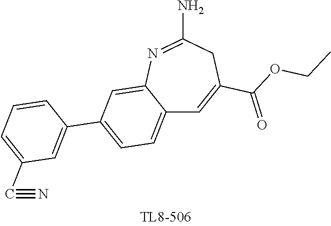 [SMILES: CCOC(=O)C1=Cc2ccc(-c3cccc(C#N)c3)cc2N=C(N)C1]

In certain embodiments, an activator of innate immune response is PF-4878691, isatoribine, SM-324405, SM-324406, AZ12441970, AZ12443988, GSK-2245035, RG7854, GS-9620, LHC165, NKTR-262, GS-9688, VTX-2337, or pharmaceutically acceptable salts thereof. PF-4878691, isatoribine, SM-324405, SM-324406, AZ12441970, AZ12443988, GSK-2245035, RG7854, and GS-9620 are TLR7 agonists. LHC165 and NKTR-262 are agonists of both TLR7 and TLR8 agonists. GS-9688 and VTX-2337 are TLR8 agonists.

In certain embodiments, an activator of innate immune response is an imidazoquinoline derivative, including dactolisib, imiquimod, gardiquimod, resiquimod, sumanirole, and pharmaceutically acceptable salts thereof.

In certain embodiments, an activator of innate immune response is CL097, or a pharmaceutically acceptable salt thereof. CL097 is a highly water-soluble derivative resiquimod (≥20 mg/ml). CL097 is a TLR7 and TLR8 ligand. It induces the activation of NF-κB at 0.4 μM (0.1 μg/ml) in TLR7-transfected HEK293 cells and at 4 μM (1 μg/ml) in TLR8-transfected HEK293 cells.

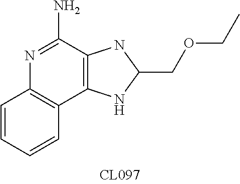 [SMILES: CCOCC1Nc2c(N)nc3ccccc3c2N1]

In certain embodiments, an activator of innate immune response is CL075, or a pharmaceutically acceptable salt thereof. CL075 (3M002) is a thiazoloquinolone derivative that stimulates TLR8 in human peripheral blood mononuclear cells. It activates NF-κB and triggers preferentially the production of TNF-α and IL-12. CL075 also induces the secretion of IFN-α through TLR7, but to a lesser extent. It induces the activation of NF-κB at 0.4 μM (0.1 μg/ml) in TLR8-transfected HEK293 cells, and ˜ 10 times more CL075 is required to activate NF-κB in TLR7-transfected HEK293 cells.

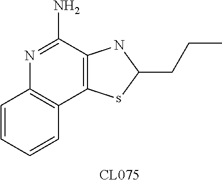

In certain embodiments, an activator of innate immune response is MEDI9197, or a pharmaceutically acceptable salt thereof. MEDI9197 (3M052) is an injectable TLR7 and TLR8 agonist. It is an imidazoquinoline immune response modifier (IRM) bearing a C18 lipid moiety and designed for slow dissemination from the site of application.

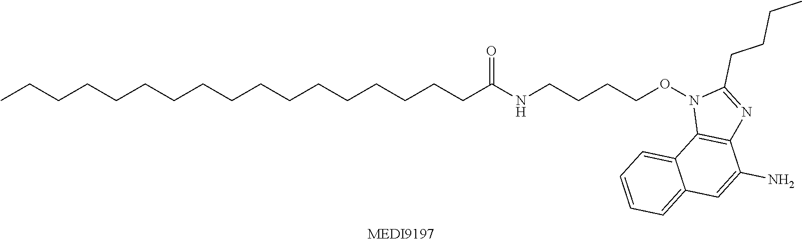 [SMILES: CCCCCCCCCCCCCCCCCC(=O)NCCCCOn1c(CCCC)nc2c(N)cc3ccccc3c21]

In certain embodiments, an activator of innate immune response is resiquimod (R848), or a pharmaceutically acceptable salt thereof. In particular, resiquimod is an agent that acts as an immune response modifier and has antiviral and antitumor activity. It is used as a topical gel in the treatment of skin lesions such as those caused by the herpes simplex virus and cutaneous T cell lymphoma. It is also used as an adjuvant to increase the effectiveness of vaccines. It has several mechanisms of action, being both an agonist for toll-like receptor 7 (TLR7) and 8 (TLR8), and an upregulator of the opioid growth factor receptor.

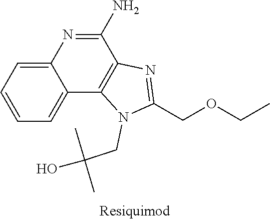 [SMILES: CCOCc1nc2c(N)nc3ccccc3c2n1CC(C)(C)O]

In certain embodiments, an activator of innate immune response is a TLR7-selective antedrug. In certain embodiments, an activator of innate immune response is SM-324405, AZ12441970, or pharmaceutically acceptable salts thereof.

In certain embodiments, an activator of innate immune response is GS-9620. In certain embodiments, an activator of innate immune response is PF-4878691. In certain embodiments, an activator of innate immune response is NKTR-262. In certain embodiments, an activator of innate immune response is LHC165.

In certain embodiments, an activator of innate immune response is an inflammasome inducer. Inflammasomes are multimeric protein complexes that are crucial for host defense to infection and endogenous danger signals. They promote the secretion of the pro-inflammatory cytokines interleukin (IL)-1B and IL-18 and cause a rapid and pro-inflammatory form of cell death called pyroptosis.

In certain embodiments, an activator of innate immune response is an inducer of NLRP3, AIM2, NLRC4, or NLRP1 inflammasomes.

In certain embodiments, an activator of innate immune response is

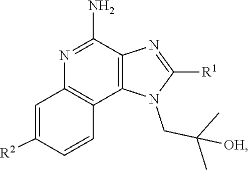

or a pharmaceutically acceptable salt thereof, wherein: R₁ is H, and R₂ is H; R₁ is a butyl group and R² is H; R¹ is H and R² is —CO₂CH₃; or R¹ is a butyl group and R² is —CO₂CH₃.

In certain embodiments, an activator of innate immune response is an imadazoquinoline; an imidazonaphthyridine; a pyrazolopyridine; an aryl-substituted imidazoquinoline; a compound having a 1-alkoxy 1H-imidazo ring system; an oxazolo[4,5-c]-quinolin-4-amine; a thiazolo[4,5-c]-quinolin-4-amine; a selenazolo [4,5-c]-quinolin-4-amine; an imidazonaphthyridine; an imidazoquinolinamine; a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1-yl)-5-methyl-1-oxo-1H,5H-benzo[ij]quinolizine-2-carboxylic acid; a pyridoquinoxaline-6-carboxylic acid; a 6,7-dihydro-8-(imidazol-1-yl)-5-methyl-1-oxo-1H,5H-benzo[ij]quinolizine-2-carboxylic acid; a substituted naphtho [ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a 7-hydroxy-benzo[ij]quinolizine-2-carboxylic acid; a substituted pyrido[1,2,3,-de]-1,4-benzoxazine; a N-methylene malonate of tetrahydroquinoline, and/or pharmaceutically acceptable salts thereof.

In certain embodiments, an activator of innate immune response is any NLRP3 agonist disclosed in U.S. patent application U.S. Ser. No. 15/253,215, filed Aug. 31, 2016, the entire contents of which are incorporated herein by reference.

In certain embodiments, an activator of innate immune response is a RORγ agonist. A RORγ agonist is an agent that promotes RORγ activity, such as by binding to and activating RORγ or by increasing expression of RORγ in a patient or population of cells. The RORγ agonist may be, for example, a small organic molecule, polypeptide, or nucleic acid. Various RORγ agonists are reported in the literature, such as in U.S. Pat. No. 9,394,315; Zhang et al. in Mol. Pharmacol. (2012) vol. 82, pages 583-590; and Wang et al. in ACS Chem. Biol. (2010), vol. 5, pages 1029-1034; each of which is hereby incorporated by reference for the purpose described herein.

In certain embodiments, an activator of innate immune response is a RORγ agonist such as

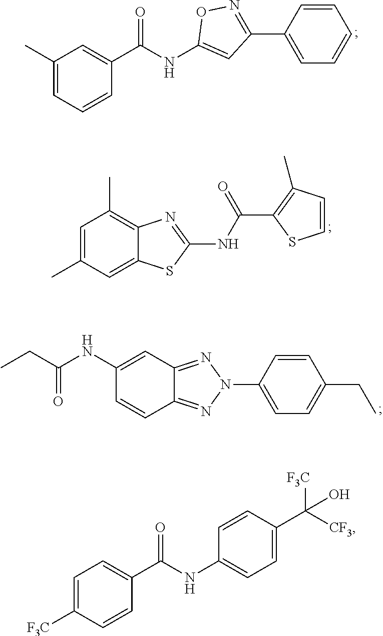 [SMILES: Cc1cccc(C(=O)Nc2cc(-c3ccccc3)no2)c1 ; Cc1cc(C)c2nc(NC(=O)c3sccc3C)sc2c1 ; CCC(=O)Nc1ccc2nn(-c3ccc(CC)cc3)nc2c1 ; O=C(Nc1ccc(C(O)(C(F)(F)F)C(F)(F)F)cc1)c1ccc(C(F)(F)F)cc1]

and pharmaceutically acceptable salts thereof.

In certain embodiments, an activator of innate immune response is a generic or specific compound described in U.S. Pat. No. 9,394,315, granted Jul. 19, 2016, the entire contents of which are incorporated herein by reference, such as a compound of Formula (I):

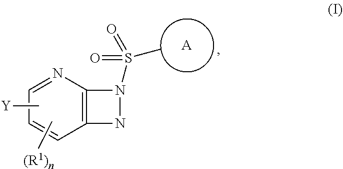

or a pharmaceutically acceptable salt thereof, wherein:

A is aryl, aralkyl, heteroaryl, cycloalkyl, or heterocycloalkyl; each of which is optionally substituted with 1, 2, or 3 substituents independently selected from the group consisting of halogen, hydroxyl, C₁₋₆alkyl, C₁₋₆haloalkyl, C₁₋₆hydroxyalkyl, C₁₋₆alkoxy, C₁₋₆haloalkoxy, —N(R⁴)(R⁵), —CO₂R⁶, —C(O)R⁶, —CN, —C₁₋₄alkylene-C₁₋₄alkoxy, —C₁₋₄alkylene-N(R⁴)(R⁵), C₁₋₄alkylene-CO₂R⁶, —O—C₁₋₆alkylene-N(R⁴)(R⁵), —N(R⁴)C(O)—C₁₋₆alkylene-N(R⁴)(R⁵), —S(O)ₚC₁₋₆alkyl, —SO₂N(R⁴)(R⁵), —N(R⁴)SO₂(C₁₋₆alkyl), —C(O)N(R⁴)(R⁵), and —N(R⁴)C(O)N(R⁴)(R⁵);
X is —O—[C(R⁶)(R⁷)]—[C(R⁶)₂]ₘ—Ψ, —O—C(R⁶)₂—C(R⁶)(R⁷)—C(R⁶)₂—Ψ, —O—C(R⁶)₂—C(R⁶)(R⁷)—Ψ, —C(R⁶)₂—[C(R⁶)(R⁷)]—[C(R⁶)₂m-Ψ, —C(O)—[C(R⁶)(R⁷)]—[C(R⁶)₂]m-Ψ, —C(R⁶)₂—N(R⁸)[C(R⁶)(R⁷)]—[C(R⁶)₂]m-Ψ, —C(R⁶)═N—Ψ, —C(R⁶)₂C(R⁶)═N—Ψ, —N═C(R⁶)—Ψ, or —N═C(R⁶)C(R⁶)₂—Ψ; wherein Ψ is a bond to the sulfonamide ring nitrogen atom in Formula I;
Y is —N(R²)(R³) or —O-aralkyl, wherein said aralkyl is optionally substituted with 1, 2, or 3 substituents independently selected from the group consisting of halogen, hydroxyl, C₁₋₆alkoxy, C₁₋₆haloalkoxy, C₁₋₆alkyl, C₁₋₆haloalkyl, —N(R⁴)(R⁵), —CN, —CO₂—C₁₋₆alkyl, —C(O)—C₁₋₆alkyl, —C(O)N(R⁴)(R⁵), —S(O)ₚC₁₋₆alkyl, —SO₂N(R⁴)(R⁵), and —N(R⁴)SO₂(C₁₋₆alkyl);
R¹ represents independently for each occurrence hydrogen, halogen, or C₁₋₆alkyl;
R² is —C(O)-aryl, —C(O)-aralkyl, —C(O)—[C(R⁶)₂]ₘ-cycloalkyl, —C(O)—[C(R⁶)₂]ₘ-heterocyclyl, —C(O)—C₁₋₆alkyl, —C(O)—C₁₋₆alkylene-C₁₋₆alkoxyl, —C(O)—C₁₋₆alkylene-cycloalkyl, or —C(O)—C₁₋₆alkylene-heterocycloalkyl; each of which is optionally substituted with 1, 2, or 3 substituents independently selected from the group consisting of halogen, hydroxyl, C₁₋₆alkoxy, C₁₋₆haloalkoxy, C₁₋₆alkyl, C₁₋₆haloalkyl, —N(R⁴)(R⁵), —CN, —CO₂—C₁₋₆alkyl, —C(O)—C₁₋₆alkyl, —C(O)N(R⁴)(R⁵), —S(O)ₚC₁₋₆alkyl, —SO₂N(R⁴)(R⁵), and —N(R⁴)SO₂(C₁₋₆alkyl);
R³ is hydrogen or C₁₋₆alkyl;
R⁴ and R⁵ each represent independently for each occurrence hydrogen or C₁₋₆alkyl; or R⁴ and R⁵ taken together with the nitrogen atom to which they are attached form a 3-7 membered heterocyclic ring;
R⁶ represents independently for each occurrence hydrogen or C₁₋₆alkyl;
R⁷ is hydrogen, hydroxyl, C₁₋₆hydroxyalkyl, C₁₋₆alkyl, C₁₋₆haloalkyl, —CO₂R⁶, C₁₋₆alkylene-CO₂R⁶, C₁₋₄hydroxyalkylene-CO₂R⁶, —N(R⁴)(R⁵), C₁₋₆alkylene-N(R⁴)(R⁵), C₁₋₆hydroxyalkylene-N(R⁴)(R⁵), —N(R⁴)C(O)R⁹, C₁₋₆alkylene-N(R⁴)C(O)R⁹, C₁₋₆alkylene-C(O)N(R⁴)(R⁵), —N(R⁴)CO₂—C₁₋₆alkyl, or C₁₋₆alkylene-N(R⁴) (C(O)N(R⁴)(R⁵); or R⁷ is heterocycloalkyl or C₁₋₄alkylene-heterocycloalkyl, wherein the heterocycloalkyl is optionally substituted with 1, 2, or 3 substituents independently selected from the group consisting of oxo, halogen, hydroxyl, C₁₋₆alkyl, C₁₋₆haloalkyl, C₁₋₆hydroxyalkyl, C₁₋₆alkoxy, and C₁₋₆haloalkoxy;
R⁸ is hydrogen, C₁₋₆alkyl, or —C(O)—C₁₋₆alkyl;
R⁹ is hydrogen, C₁₋₆alkyl, C₁₋₆hydroxyalkyl, C₁₋₆alkylene-N(R⁴)(R⁵), or C₁₋₆alkylene-N(R⁴)C(O)—C₁₋₆alkyl;
n is 1 or 2; and
m and p each represent independently for each occurrence 0, 1, or 2.

In certain embodiments, an activator of innate immune response is any RORγ agonist disclosed in U.S. Pat. No. 9,394,315, granted Jul. 19, 2016, the entire contents of which are incorporated herein by reference. In certain embodiments, an activator of innate immune response is any RORγ agonist disclosed in U.S. Pat. No. 10,532,088, granted Jan. 14, 2020, the entire contents of which are incorporated herein by reference.

In certain embodiments, an activator of innate immune response is a RIG-I-like receptor (RLR) agonist. In certain embodiments, an activator of innate immune response is RGT-100.

### Modulators of Adaptive Immunity

In certain embodiments, compositions described herein comprise nucleic acids that encode or act as modulator(s) of the adaptive immune response.

The adaptive immune response system, also known as the acquired immune system, is a subsystem of the overall immune system that includes highly specialized systemic cells and processes that eliminate or prevent pathogen growth. The adaptive immune system is one of the two main immunity strategies found in vertebrates (the other being the innate immune system). Adaptive immunity creates immunological memory after an initial response to a specific pathogen and leads to an enhanced response to subsequent encounters with that pathogen. This process of acquired immunity is the basis of vaccination. Like the innate system, the adaptive system includes both humoral immunity components and cell-mediated immunity components. Unlike the innate immune system, the adaptive immune system is highly specific to a particular pathogen.

The adaptive immune response system is triggered in vertebrates when a pathogen evades the innate immune response system, generates a threshold level of antigen, and generates “stranger” or “danger” signals activating dendritic cells. The major functions of the acquired immune system include recognition of specific “non-self” antigens in the presence of “self” during the process of antigen presentation; generation of responses that are tailored to eliminate specific pathogens or pathogen-infected cells; and development of immunological memory, in which pathogens are “remembered” through memory B cells and memory T cells.

### Activators of the Adaptive Immune Response

In some embodiments, compositions provided herein may comprise one or more nucleic acids encoding or acting as activators of the adaptive immune response. In some embodiments, activators of the adaptive immune response are or comprise nucleic acids which may act directly to activate the adaptive immune system and/or encode an activator of the adaptive immune system.

Useful approaches to activating the adaptive immune response system (e.g., activating therapeutic antitumor immunity) include the blockade of immune checkpoints. Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. Cytotoxic T-lymphocyte-associated antigen 4 (CTLA-4) antibodies were the first of this class of immunotherapeutics to receive FDA approval (ipilimumab). Preliminary clinical findings with blockers of additional immune-checkpoint proteins, such as programmed cell death protein 1 (PD-1), indicate broad and diverse opportunities to enhance antitumor immunity with the potential to produce durable clinical responses.

PD-1, functioning as an immune checkpoint, plays an important role in down-regulating the immune system by preventing the activation of T cells, which in turn reduces autoimmunity and promotes self-tolerance. The inhibitory effect of PD-1 is accomplished through a dual mechanism of promoting apoptosis (programmed cell death) in antigen-specific T cells in lymph nodes while simultaneously reducing apoptosis in regulatory T cells (suppressor T cells). A new class of therapeutics that block PD-1, the PD-1 inhibitors (e.g., anti-PD-1 antibodies), activate the immune system to attack tumors and are therefore used to treat some types of cancer. In addition, antibodies of Programmed death-ligand 1 (PD-L1) provide a similar impact on activating the adaptive immune response as antibodies targeting PD-1. Accordingly, compositions comprising anti-PD-L1 antibodies are expected to provide a similar therapeutic effect as those comprising anti-PD-1 antibodies.

In certain embodiments, an activator of adaptive immune response is a small molecule. In certain embodiments, an activator of adaptive immune response is a biologic. In certain embodiments, the biologic and/or small molecule is a nucleic acid, or is an RNA or protein product encoded by a nucleic acid. In certain embodiments, the biologic is a protein. In certain embodiments, the biologic is an antibody or fragment thereof. In certain embodiments, the biologic is a nucleic acid that encodes a protein or RNA which can act as an activator of the adaptive immune response.

In certain embodiments, provided compositions comprise at least one nucleic acid encoding an antibody. In certain embodiments, provided compositions comprise at least one nucleic acid and optionally include an antibody. In certain embodiments, the nucleic acid encodes or the antibody is an activator of adaptive immune response, wherein the encoded activator is an antibody. In certain embodiments, an activator of adaptive immunity is a nucleic acid encoding, anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD3 antibody, an anti-CD27 antibody, an anti-CD28 antibody, an anti-CD28H antibody, an anti-CD30 antibody, an anti-CD39 antibody, an anti-CD40 antibody, an anti-CD43 antibody, an anti-CD47 antibody, an anti-CD48 antibody, an anti-CD70 antibody, an anti-CD73 antibody, an anti-CD96 antibody, an anti-CD123 antibody, an anti-CD155 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAMI antibody, an anti-BTLA antibody, an anti-LIGHT antibody, an anti-TIGIT antibody, an anti-VISTA antibody, an anti-HVEM antibody, an anti-Siglec antibody, an anti-GALI antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-CEACAMI antibody, an anti-CEACAM5 antibody, an anti-CEACAM6 antibody, an anti-MICA antibody, an anti-MICB antibody, an anti-NKG2D antibody, an anti-NKG2A antibody, an anti-A2AR antibody, an anti-C₅aR antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, an anti-Tie2 antibody, an anti-CCR4 antibody, an anti-TRAIL-DR5 antibody, and/or any combination thereof.

In certain embodiments, an activator of adaptive immune response comprises at least one nucleic acid encoding a fragment of any of the antibodies listed herein, and/or a fragment of any of the antibodies listed herein. In certain embodiments. In certain embodiments, an activator of adaptive immune response is a nucleic acid encoding a humanized form of any of the antibodies listed herein. In certain embodiments, an activator of adaptive immune response is a nucleic acid encoding a single chain of any of the antibodies listed herein. In certain embodiments, an activator of immune response is a nucleic acid encoding a multimeric form of any of the antibodies listed herein (e.g., dimeric IgA molecules, pentavalent IgM molecules).

In certain embodiments, an activator of adaptive immune response is a nucleic acid encoding or is an anti-PD-1 antibody, an agonist anti-CD137 antibody, an agonist anti-CD40 antibody, an anti-CTLA-4 antibody, an anti-LAG-3 antibody, an anti-TIM3, or a combination thereof. In certain embodiments, an activator of adaptive immune response is a nucleic acid encoding anti-PD-1 antibody or an anti-CTLA-4 antibody. In certain embodiments, an activator of adaptive immune response is a nucleic acid encoding anti-PD-1 antibody. In certain embodiments, an activator of adaptive immune response is a nucleic acid encoding anti-CTLA-4 antibody. In certain embodiments, an activator of adaptive immune response is a nucleic acid encoding agonist anti-CD137 antibody. In certain embodiments, an activator of adaptive immune response is a nucleic acid encoding anti-LAG-3 antibody. In certain embodiments, an activator of adaptive immune response is a nucleic acid encoding anti-TIM3 antibody.

In certain embodiments, an activator of adaptive immune response is a nucleic acid encoding an antibody, and/or is an antibody. In certain embodiments, the nucleic acid encodes or the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, IPH5401, IPH4102, IPH4301, IPH52, IPH53, varlilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, IMC-CS4, emactuzumab, trastuzumab, pertuzumab, obinutuzumab, cabiralizumab, margetuximab, enoblituzumab, mogamulizumab, panitumumab, carlumab, ramucirumab, bevacizumab, rituximab, cetuximab, fresolimumab, denosumab, MGA012, AGEN1884, AGEN2034, LY3300054, JTX-4014, teplizumab, FPA150, PF-04136309, PF-06747143, AZD5069, GSK3359609, FAZ053, TSR022, MBG453, REGN2810, REGN3767, MOXR0916, PF-04518600, RO7009789, BMS986156, GWN323, JTX-2011, NKTR-214, GSK3174998, DS-8273a, NIS793, BGB-A317, and/or any combination thereof.

In certain embodiments, an activator of adaptive immune response is a nucleic acid encoding an antibody, and/or is an antibody. In certain embodiments, the nucleic acid encodes or the antibody is pembrolizumab, nivolumab, pidilizumab, ipilimumab, tremelimumab, durvalumab, atezolizumab, REGN2810, MGA012, AGEN1884, AGEN2034, LY3300054, JTX-4014, avelumab, and/or any combination thereof.

In certain embodiments, an activator of adaptive immune response is an antibody mimetic or antibody fusion, or a nucleic acid encoding either an antibody mimetic or antibody fusion.

In certain embodiments, an activator of adaptive immune response is a nucleic acid encoding a bispecific antibody, and/or is a bispecific antibody. In certain embodiments, the nucleic acid encodes or the bispecific antibody is RG7802 (antibody targeting carcinoembryonic antigen (CEA) and the CD3 receptor), RG7828 (a bispecific monoclonal antibody that targets CD20 on B cells and CD3 on T cells), RG7221 (a bispecific monoclonal antibody that targets VEGF and angiopoietin 2), RG7386 (a bispecific monoclonal antibody that targets FAP and DR5), ERY974 (a bispecific monoclonal antibody that targets CD3 and glypican-3), MGD012 (a bispecific monoclonal antibody that targets PD-1 and LAG-3), AMG211 (a bispecific T cell engager that targets CD3 and CEA), MEDI573 (a bispecific monoclonal antibody that targets IGF1 and IGF2), MEDI565 (a bispecific monoclonal antibody that targets CD3 and CEA), FS17 (undisclosed targets), FS18 (a bispecific monoclonal antibody that targets LAG3 and an undisclosed target), FS20 (undisclosed targets), FS22 (undisclosed targets), FS101 (a bispecific monoclonal antibody that targets EGFR and HGF), FS117 (undisclosed targets), FS118 (a bispecific monoclonal antibody that targets LAG3 and PD-L1), RO6958688 (a bispecific monoclonal antibody that targets CD3 and CEA), MCLA-128 (a bispecific monoclonal antibody that targets HER2 and HER3), M7824 (bi-functional fusion-protein targeting PD-L1 and TGFβ), MGD009 (a humanized antibody that recognizes both B7-H3 and CD3), MGD013 (a bispecific PD-1 and LAG-3 antibody), and/or any combinations thereof.

In certain embodiments, an activator of adaptive immune response is an antibody-drug conjugate. In certain embodiments, an antibody-drug conjugate is trastuzumab emtansine, inotuzumab ozogamicin, PF-06647020, PF-06647263, PF-06650808, RG7596, RG7841, RG7882, RG7986, DS-8201, ABBV-399, glembatumumab vedotin, inotuzumab ozogamicin, MEDI4276, or pharmaceutically acceptable salts thereof.

In certain embodiments, an activator of adaptive immune response is a small molecule. In certain embodiments, the small molecule is an IDO inhibitor, a TGFβR inhibitor, a BRAF inhibitor, a KIT inhibitor, an A2aR inhibitor, a Tie2 inhibitor, an arginase inhibitor, an iNOS inhibitor, an HIFla inhibitor, a STAT3 inhibitor, a PGE2 inhibitor, a PDE5 inhibitor, a RON inhibitor, an mTOR inhibitor, a JAK2 inhibitor, a HSP90 inhibitor, a PI3K-AKT inhibitor, a B-catenin inhibitor, a GSK3B inhibitor, an IAP inhibitor, an HDAC inhibitor, a DNMT inhibitor, a BET inhibitor, a COX2 inhibitor, a PDGFR inhibitor, a VEGFR inhibitor, a BCR-ABL inhibitor, a proteasome inhibitor, an angiogenesis inhibitor, a MEK inhibitor, a BRAF+MEK inhibitor, a pan-RAF inhibitor, an EGFR inhibitor, a PARP inhibitor, a glutaminase inhibitor, a WNT inhibitor, a FAK inhibitor, an ALK inhibitor, a CDK4/6 inhibitor, or an FGFR3 inhibitor.

In certain embodiments, the small molecule is celecoxib, sunitinib, imatinib, vemurafenib, dabrafenib, bortezomib, vorinostat, pomalidomide, thalidomide, lenalidomide, epacadostat, indoximid, GDC0919, BMS986205, AZD8055, AZD4635, CPI-444, PBF509, LCL161, CB-839, CB-1158, FPA008, BLZ945, IPI-549, pexidartinib, galunisertib, birinapant, trametinib, cobimetinib, binimetinib, ensartib, gefitinib, pazopanib, sorafenib, nintedanib, SYM004, veliparib, olaparib, BGB-290, everolimus, LXH254, azacitidine, decitabine, guadecitabine, RRX001, CC486, romidepsin, entinostat, panobinostat, tamoxifen, ibrutinib, idelalisib, capmatinib, selumetinib, abemaciclib, palbociclib, glasdegib, enzalutamide, AZD9150, PF-06840003, SRF231, Hu5F9-G4, CC-900002, TTI-621, WNT974, BGJ398, LY2874455, or pharmaceutically acceptable salts thereof.

### Additional Therapeutic Agents

In certain embodiments, provided compositions may comprise nucleic acids acting as or encoding therapeutic agents not specifically disclosed in sections “Modulators of the innate immune response” or “Modulators of adaptive immunity”.

In certain embodiments, provided compositions may comprise at least one nucleic acid acting as or encoding a modulator of macrophage effector function. Macrophages are immune cells that are derived from circulating monocytes, reside in all tissues, and participate in many states of pathology. Macrophages play a dichotomous role in cancer, where they can promote tumor growth but also can serve as critical immune effectors of therapeutic antibodies. Macrophages express all classes of Fcγ receptors, and they have potential to destroy tumors via the process of antibody-dependent cellular phagocytosis. A number of studies have demonstrated that macrophage phagocytosis is a major mechanism of action of many antibodies approved to treat cancer. Consequently, a number of approaches to augment macrophage responses to therapeutic antibodies are under investigation, including the exploration of new targets and development of antibodies with enhanced functions. The response of macrophages to antibody therapies can also be enhanced with engineered Fc variants, bispecific antibodies, or antibody-drug conjugates. Macrophages have demonstrated success as effectors of cancer immunotherapy.

In certain embodiments, provided compositions may comprise at least one nucleic acid acting as or encoding a modulator of macrophage effector function, wherein the modulator of macrophage effector function is a modulator of suppressive myeloid cells, including myeloid-derived suppressor cells (MDSCs). In certain embodiments, the modulator of macrophage effector function may kill, deplete, or potentiate macrophages and/or MDSCs. In certain embodiments, the modulator of macrophage effector function is an anti-CD40 antibody, an anti-CD47 antibody, an anti-CSF1 antibody, or an anti-CSFIR antibody. In certain embodiments, the modulator of macrophage effector function is SRF231, Hu5F9-G4, CC-900002, or TTI-621 (anti-CD47 antibodies). In certain embodiments, the modulator of macrophage effector function is MCS-110 (an anti-CSF1 antibody). In certain embodiments, the modulator of macrophage effector function is FPA008, RG7155, IMC-CS4, AMG820, or UCB6352 (anti-CSFIR antibodies).

In some embodiments, provided compositions may comprise at least one nucleic acid acting as or encoding a modulator of effector function and optionally an additional small molecule. In certain embodiments, the optional modulator of macrophage effector function is a small molecule inhibitor of CSFIR. In certain embodiments, the modulator of macrophage effector function is BLZ945, GW2580, or PLX3397 (small molecule inhibitors of CSFIR).

In some embodiments, provided compositions may comprise at least a biomaterial and at least one nucleic acid acting as or encoding a modulator of effector function, wherein the modulator is a BTK inhibitor, an ITK inhibitor, a PI3Ky inhibitor, or a PI3K8 inhibitor.

In certain embodiments, provided compositions comprising at least a biomaterial and a nucleic acid may further comprise an oncolytic virus. In certain embodiments, the oncolytic virus includes, but is not limited to, herpes simplex viruses (e.g., HSV1716, Onco Vex GM-CSF); adenoviruses (e.g., H101, Onyx-15); polioviruses (e.g., PV1(RIPO)); reoviruses (e.g., reolysin); senecaviruses (e.g., NTX-010, SVV-001); Rigvir virus; maraba virus; measles; Newcastle disease virus; vaccinia; or ECHO virus.

In certain embodiments, provided compositions comprising at least a biomaterial and a nucleic acid may further comprise a radioactive isotope (e.g., as part of a molecule or on a bead). In certain embodiments the radioactive isotope is Yttrium-90, Palladium-103, Iodine-125, Cesium 131, or Iridium 192.

In certain embodiments, provided compositions comprise at least a biomaterial and a nucleic acid which may be a chemotherapeutic agent. In certain embodiments, provided compositions comprise at least a biomaterial, a nucleic acid, and an additional optional chemotherapeutic agent. In certain embodiments, the chemotherapeutic agent includes, but is not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine and temozolomide), platinum-containing compounds (e.g., cisplatin, carboplatin, and oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, and mytomycin C), anti-metabolites, DHFR inhibitors (e.g., methotrexate, dichloromethotrexate, trimetrexate, and edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, and capecitabine), cytosine analogs (e.g., cytarabine (ara C), cytosine arabinoside, and fludarabine), purine analogs (e.g., mercaptopurine and thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, and peplomycin), anthracycline (e.g., daunorubicin, doxorubicin, pegylated liposomal doxorubicin, idarubicin, epirubicin, pirarubicin, zorubicin, and mitoxantrone), MDR inhibitors (e.g., verapamil), Ca²⁺ ATPase inhibitors (e.g., thapsigargin), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, hexamethyl melamine, and/or pharmaceutically acceptable salts thereof.

In certain embodiments, the chemotherapeutic agent is an immunomodulatory chemotherapeutic agent. In certain embodiments, the chemotherapeutic agent has known immunomodulatory function (e.g., induction of immunogenic cell death or depletion of immunosuppressive regulatory immune cells). In certain embodiments, the chemotherapeutic agent is included in the drug delivery compositions due to its immunotherapeutic properties rather than its use as a conventional cancer-cell intrinsic cytotoxic chemotherapy. In certain embodiments, the drug delivery compositions do not comprise a chemotherapeutic agent. In certain embodiments, the drug delivery compositions do not comprise a cytotoxic agent.

In some embodiments, provided compositions may comprise at least a biomaterial and at least one nucleic acid acting as or encoding a targeted agent. In certain embodiments, the targeted agent includes, but is not limited to, an IDO inhibitor, a TGFβR inhibitor, an arginase inhibitor, an iNOS inhibitor, a HIF1α inhibitor, a STAT3 inhibitor, a CSFIR inhibitor, a PGE2 inhibitor, a PDE5 inhibitor, a RON inhibitor, an mTOR inhibitor, a JAK2 inhibitor, an HSP90 inhibitor, a PI3K-AKT inhibitor, a β-catenin inhibitor, a GSK3β inhibitor, an IAP inhibitor, an HDAC inhibitor, a DNMT inhibitor, a BET inhibitor, an A2AR inhibitor, a BRAF+MEK inhibitor, a pan-RAF inhibitor, a PI3Ky inhibitor, a PI3K8 inhibitor, an EGFR inhibitor, a VEGF inhibitor, a PARP inhibitor, a glutaminase inhibitor, a BTK inhibitor, an ITK inhibitor, a WNT inhibitor, a FAK inhibitor, an ALK inhibitor, a CDK4/6 inhibitor, a or an FGFR3 inhibitor.

In some embodiments, provided compositions may comprise at least a biomaterial and at least one nucleic acid acting as or encoding a targeted agent. In some embodiments, provided compositions may comprise at least a biomaterial and at least one nucleic acid acting as or encoding a targeted agent, and optionally an additional targeted agent. In some embodiments, a targeted agent includes, but is not limited to, imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEFR), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), epacadostat, indoximid, GDC0919, BMS986205, AZD4635, CPI-444, PBF509, LCL161, CB-839, CB-1158, FPA008, BLZ945, IPI-549, pexidartinib, galunisertib, birinapant, trametinib, dabrafenib, vemurafenib, cobimetinib, binimetinib, ensartib, pazopanib, nintedanib, SYM004, veliparib, olaparib, BGB-290, LXH254, azacitidine, decitabine, guadecitabine, RRX001, CC486, romidepsin, entinostat, vorinostat, panobinostat, tamoxifen, ibrutinib, idelalisib, capmatinib, selumetinib, abemaciclib, palbociclib, glasdegib, enzalutamide, AZD9150, PF-06840003, SRF231, Hu5F9-G4, CC-900002, TTI-621, WNT974, BGJ398, LY2874455, an anti-Tie2 antibody, or pharmaceutically acceptable salts thereof.

### Embodiments of Drug Delivery Compositions

In some embodiments, provided compositions comprise at least one polymeric biomaterial, and at least one nucleic acid. In certain embodiments, the provided composition comprises a multipolymeric biomaterial, and at least one nucleic acid.

In certain embodiments, provided compositions comprise polymeric biomaterial(s), and more than one nucleic acid.

In certain embodiments, provided compositions comprise polymeric biomaterial(s), one or more nucleic acids, and one or more polynucleotide carriers.

In certain embodiments, provided compositions comprise at least one polymeric biomaterial, and at least one nucleic acids encoding peptides.

In certain embodiments, provided compositions comprise polymeric biomaterial(s), at least one nucleic acid encoding a peptide, and at least one polynucleotide carrier.

In certain embodiments, provided compositions comprise polymeric biomaterial(s), one or more nucleic acids, a polynucleotide carrier, and an inhibitor of a pro-inflammatory pathway.

In certain embodiments, provided compositions comprise polymeric biomaterial(s), one or more nucleic acids, a polynucleotide carrier, an inhibitor of a pro-inflammatory pathway, and an activator of innate immune response.

In certain embodiments, provided compositions comprise polymeric biomaterial(s), one or more nucleic acids, a polynucleotide carrier, an inhibitor of a pro-inflammatory pathway, an activator of innate immune response, and an additional activator of innate immune response.

In certain embodiments, provided compositions comprise polymeric biomaterial(s), one or more nucleic acids, a polynucleotide carrier, an inhibitor of a pro-inflammatory pathway, an activator of innate immune response, and a cytokine.

In certain embodiments, provided compositions comprise polymeric biomaterial(s), one or more nucleic acids, a polynucleotide carrier, an inhibitor of a pro-inflammatory pathway, an activator of innate immune response, an additional activator of innate immune response, and a cytokine.

In certain embodiments, provided compositions comprise polymeric biomaterial(s), one or more nucleic acids, a polynucleotide carrier, an inhibitor of a pro-inflammatory pathway, an activator of innate immune response, and a chemokine.

In certain embodiments, provided compositions comprise polymeric biomaterial(s), one or more nucleic acids, a polynucleotide carrier, and a nucleic acid inhibitor or promoter of a pro-inflammatory pathway.

In certain embodiments, provided compositions comprise polymeric biomaterial(s), a nucleic acid based inhibitor or promoter of a pro-inflammatory pathway, and an activator of innate immune response.

In certain embodiments, provided compositions comprise polymeric biomaterial(s), a nucleic acid based inhibitor or promoter of the innate immune response, an inhibitor of a pro-inflammatory pathway, and an activator of innate immune

In certain embodiments, provided compositions comprise polymeric biomaterial(s), a nucleic acid encoding a peptide, an inhibitor of a pro-inflammatory pathway, an activator of innate immune response, and a cytokine.

In certain embodiments, provided compositions comprise polymeric biomaterial(s), a nucleic acid encoding a peptide, an inhibitor of a pro-inflammatory pathway, an activator of innate immune response, and a cytokine.

In certain embodiments, provided compositions comprise polymeric biomaterial(s), a nucleic acid encoding a peptide, an inhibitor of a pro-inflammatory pathway, an activator of innate immune response, and a chemokine.

In certain embodiments, provided compositions comprise polymeric biomaterial(s), an inhibitor of a pro-inflammatory pathway, an activator of innate immune response, an additional activator of innate immune response, and a chemokine; wherein at least one of the components is or is encoded by a nucleic acid.

In certain embodiments, provided compositions comprise polymeric biomaterial(s), an inhibitor of a pro-inflammatory pathway, and a cytokine; wherein at least one of the components is or is encoded by a nucleic acid.

In certain embodiments, provided compositions comprise polymeric biomaterial(s), an inhibitor of a pro-inflammatory pathway, and a chemokine; wherein at least one of the components is or is encoded by a nucleic acid.

In certain embodiments, provided compositions comprise polymeric biomaterial(s), an inhibitor of a pro-inflammatory pathway, and an activator of adaptive immune response;

wherein at least one of the components is or is encoded by a nucleic acid.

In certain embodiments, provided compositions comprise polymeric biomaterial(s), an inhibitor of a pro-inflammatory pathway, an activator of innate immune response, and an activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, an additional activator of innate immune response, and an activator of adaptive immune response; wherein at least one of the components is or is encoded by a nucleic acid.

In certain embodiments, provided compositions comprise polymeric biomaterial(s), an inhibitor of a pro-inflammatory pathway, an activator of adaptive immune response, and an additional activator of adaptive immune response; wherein at least one of the components is or is encoded by a nucleic acid.

In certain embodiments, provided compositions comprise polymeric biomaterial(s), an inhibitor of a pro-inflammatory pathway, an activator of adaptive immune response, and two additional activators of the adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, a cytokine, and an activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, an additional activator of innate immune response, a cytokine, and an activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, a cytokine, an activator of adaptive immune response, and an additional activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, an additional activator of innate immune response, a cytokine, an activator of adaptive immune response, and an additional activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, a chemokine, and an activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, an additional activator of innate immune response, a chemokine, and an activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, a chemokine, an activator of adaptive immune response, and an additional activator of adaptive immune response; wherein at least one of the components is or is encoded by a nucleic acid.

In certain embodiments, provided compositions comprise polymeric biomaterial(s), an inhibitor of a pro-inflammatory pathway, an activator of innate immune response, an additional activator of innate immune response, a chemokine, an activator of adaptive immune response, and an additional activator of adaptive immune response.

In certain embodiments, provided compositions comprise polymeric biomaterial(s), an inhibitor of a pro-inflammatory pathway, a cytokine, and an activator of adaptive immune response; wherein at least one of the components is or is encoded by a nucleic acid.

In certain embodiments, provided compositions comprise polymeric biomaterial(s), an inhibitor of a pro-inflammatory pathway, a cytokine, an activator of adaptive immune response, and an additional activator of adaptive immune response; wherein at least one of the components is or is encoded by a nucleic acid.

In certain embodiments, provided compositions comprise polymeric biomaterial(s), an inhibitor of a pro-inflammatory pathway, a chemokine, and an activator of adaptive immune response; wherein at least one of the components is or is encoded by a nucleic acid.

In certain embodiments, provided compositions comprise polymeric biomaterial(s), an inhibitor of a pro-inflammatory pathway, a chemokine, an activator of adaptive immune response, and an additional activator of adaptive immune response; wherein at least one of the components is or is encoded by a nucleic acid.

In certain embodiments, provided compositions comprise polymeric biomaterial(s), and a nucleic acid that activates the immune response.

In certain embodiments, provided compositions comprise polymeric biomaterial(s), and a nucleic acid that inhibits the immune response.

In certain embodiments, provided compositions comprise polymeric biomaterial(s), and a nucleic acid that encodes a peptide that activates the immune response.

In certain embodiments, provided compositions comprise polymeric biomaterial(s), and a nucleic acid that encodes a peptide that inhibits the immune response.

In certain embodiments, provided compositions comprise polymeric biomaterial(s), a nucleic acid that activates the immune response, and a polynucleotide carrier.

In certain embodiments, provided compositions comprise polymeric biomaterial(s), a nucleic acid that inhibits the immune response, and a polynucleotide carrier.

In certain embodiments, provided compositions comprise polymeric biomaterial(s), a nucleic acid that encodes a peptide that activates the immune response, and a polynucleotide carrier.

In certain embodiments, drug delivery comprise polymeric biomaterial(s), a nucleic acid that encodes a peptide that inhibits the immune response, and a polynucleotide carrier.

In certain embodiments, provided compositions comprise polymeric biomaterial(s), and a nucleic acid which encodes an Interleukin.

In certain embodiments, provided compositions comprise polymeric biomaterial(s), a nucleic acid which encodes an Interleukin, and a polynucleotide carrier.

In certain embodiments, provided compositions comprise polymeric biomaterial(s), and a nucleotide encoding an anti-IL-1β antibody.

In certain embodiments, provided compositions comprise polymeric biomaterial(s), and a nucleotide encoding an anti-IL-6 antibody.

In certain embodiments, provided compositions comprise polymeric biomaterial(s), and a nucleotide encoding an anti-IL-6R antibody.

In certain embodiments, provided compositions comprise polymeric biomaterial(s), and a nucleic acid encoding or acting as an inhibitor of proinflammatory immune response mediated by p38 mitogen-activated protein kinase (MAPK) pathway.

In certain embodiments, provided compositions comprise polymeric biomaterial(s), and a nucleic acid encoding a p38 MAPK inhibitor.

In certain embodiments, provided compositions comprise polymeric biomaterial(s), and a nucleic acid encoding a p38 a/B MAPK inhibitor that binds to an ATP and/or allosteric binding site of p38 MAPK.

In certain embodiments, provided compositions comprise polymeric biomaterial(s), and a nucleic acid encoding losmapimod.

In certain embodiments, provided compositions comprise polymeric biomaterial(s), and a nucleic acid encoding or acting as a TGFβR inhibitor.

In certain embodiments, provided compositions comprise polymeric biomaterial(s), and a nucleic acid encoding or acting as a CCR2 inhibitor.

In certain embodiments, provided compositions comprise polymeric biomaterial(s), and a nucleic acid encoding or acting as a CXCR4 inhibitor.

In certain embodiments, provided compositions comprise polymeric biomaterial(s), a nucleic acid encoding an anti-IL-1β antibody, and a stimulator of interferon genes (STING) agonist.

In certain embodiments, provided compositions comprise polymeric biomaterial(s), a nucleic acid encoding an anti-IL-6 antibody, and a stimulator of interferon genes (STING) agonist.

In certain embodiments, provided compositions comprise polymeric biomaterial(s), a nucleic acid encoding an anti-IL-6R antibody, and a stimulator of interferon genes (STING) agonist.

In certain embodiments, provided compositions comprise polymeric biomaterial(s), a nucleic acid encoding or acting as a TGFβR inhibitor, and a stimulator of interferon genes (STING) agonist.

In certain embodiments, provided compositions comprise polymeric biomaterial(s), a nucleic acid encoding or acting as a CCR2 inhibitor, and a stimulator of interferon genes (STING) agonist.

In certain embodiments, provided compositions comprise polymeric biomaterial(s), a nucleic acid encoding or acting as a CXCR4 inhibitor, and a stimulator of interferon genes (STING) agonist.

In certain embodiments, provided compositions comprise polymeric biomaterial(s), a nucleic acid encoding or acting as an inhibitor of proinflammatory immune response mediated by p38 mitogen-activated protein kinase (MAPK) pathway, and a TLR7/8 agonist.

In certain embodiments, provided compositions comprise polymeric biomaterial(s), a nucleic acid encoding or acting as a p38 MAPK inhibitor, and a TLR7/8 agonist.

In certain embodiments, provided compositions comprise polymeric biomaterial(s), a nucleic acid encoding a p38 a/B MAPK inhibitor that binds to an ATP and/or allosteric binding site of p38 MAPK, and a TLR7/8 agonist.

In certain embodiments, provided compositions comprise polymeric biomaterial(s), a nucleic acid encoding losmapimod, and a TLR7/8 agonist.

In certain embodiments, provided compositions comprise polymeric biomaterial(s), a nucleic acid encoding an anti-IL-1B antibody, and 2′3′-cGAMP.

In certain embodiments, provided compositions comprise polymeric biomaterial(s), a nucleic acid encoding an anti-IL-6 antibody, and 2′3′-cGAMP.

In certain embodiments, provided compositions comprise polymeric biomaterial(s), a nucleic acid encoding an anti-IL-6R antibody, and 2′3′-cGAMP.

In certain embodiments, provided compositions comprise polymeric biomaterial(s), a nucleic acid encoding an anti-IL-1B antibody, and 2′3′-c-di-AM (PS) 2 (Rp,Rp).

In certain embodiments, provided compositions comprise polymeric biomaterial(s), a nucleic acid encoding an anti-IL-6 antibody, and 2′3′-c-di-AM (PS) 2 (Rp,Rp).

In certain embodiments, provided compositions comprise polymeric biomaterial(s), a nucleic acid encoding an anti-IL-6R antibody, and 2′3′-c-di-AM (PS) 2 (Rp,Rp).

In certain embodiments, provided compositions comprise polymeric biomaterial(s), a nucleic acid encoding, an inhibitor of proinflammatory immune response mediated by p38 mitogen-activated protein kinase (MAPK) pathway, and resiquimod.

In certain embodiments, provided compositions comprise polymeric biomaterial(s), a nucleic acid encoding a p38 MAPK inhibitor, and resiquimod.

In certain embodiments, provided compositions comprise polymeric biomaterial(s), a nucleic acid encoding a p38 a/B MAPK inhibitor that binds to an ATP and/or allosteric binding site of p38 MAPK, and resiquimod.

In certain embodiments, provided compositions comprise polymeric biomaterial(s), a nucleic acid encoding losmapimod, and resiquimod.

In certain embodiments, provided compositions comprise polymeric biomaterial(s), and a nucleic acid encoding resiquimod.

In certain embodiments, provided compositions do not comprise alginate, a COX-2 inhibitor (e.g., celecoxib), and an anti-PD-1 antibody.

In certain embodiments, provided compositions do not comprise 1,3,-bis(2-chloroethyl)-1-nitrosourea (BCNU) and ethylene-vinyl acetate copolymer.

### Properties of Exemplary Compositions

In certain embodiments, the provided composition comprises at least one polymeric biomaterial described herein that forms a polymer network when exposed to any activating stimuli (e.g., temperature, concentration, crosslinking agent). In certain embodiments, polymeric biomaterials form polymer networks in less than 10 minutes. In certain embodiments, polymeric biomaterials of compositions described herein form a polymer network in less than 5 minutes, 10 minutes, 15 minutes, 20 minutes, 25 minutes, 30 minutes, 35 minutes, 40 minutes, 45 minutes, 50 minutes, 55 minutes, or one hour.

In certain embodiments, polymeric biomaterial comprised within compositions described herein form a polymer network in less than one hour. In certain embodiments, polymeric biomaterials described herein form a polymer network in less than 1 hour, 1.5 hours, 2 hours, 2.5 hours, 3 hours, 3.5 hours, or 4 hours.

In certain embodiments, polymeric biomaterial(s) of compositions described herein are liquid at the time of administration, and form a polymer network in situ or in vivo. In certain embodiments, the polymeric biomaterial forms a polymer network in vivo in less than 5 minutes, 10 minutes, 15 minutes, 20 minutes, 25 minutes, 30 minutes, 35 minutes, 40 minutes, 45 minutes, 50 minutes, 55 minutes, or one hour.

In certain embodiments, polymeric biomaterial(s) of compositions described herein are liquid at the time of administration, and form a polymer network in situ or in vivo. In certain embodiments, the polymeric biomaterial forms a polymer network in vivo in less than 1 hour, 1.5 hours, 2 hours, 2.5 hours, 3 hours, 3.5 hours, or 4 hours.

In certain embodiments, provided compositions comprising at least one polymeric biomaterial and at least one nucleic acid are characterized in that at least one nucleic acid is released from the polymeric biomaterial and is taken up by local cells. In certain embodiments, nucleic acids are released from the polymeric biomaterial and are taken up by local cells so that at least a subset of local immune cells respond to and/or express the immunomodulatory polypeptide encoded by the nucleic acid. In certain embodiments, provided compositions are characterized in that the nucleic acid is released from the polymeric biomaterial and is taken up by local cells so that at least a subset of local immune cells respond to the nucleic acid and/or express the immunomodulatory polypeptide encoded by the nucleic acid(s), and at least a subset of local immune cells have an increased expression of a type 1 interferon in response to innate immune stimulation induced by the polynucleotide agent.

In certain embodiments, provided compositions comprising at least one polymeric biomaterial and at least one nucleic acid are characterized in that a nucleic acid is released from the polymeric biomaterial and is recognized by local cells. In certain embodiments, provided compositions are characterized in that a nucleic acid is released from the polymeric biomaterial and is recognized by local cells and taken up by said cells. In certain embodiments, provided compositions are characterized in that a nucleic acid is released from the polymeric biomaterial and is taken up by local cells so that at least a subset of local immune cells respond to the polynucleotide by modulating their immune response (e.g., activation of RIG-1). In certain embodiments, provided compositions are characterized in that the polynucleotide agent is released from the polymeric biomaterial and is taken up by local cells so that at least a subset of local immune cells respond to a nucleic acid by modulating their immune response (e.g., activation of RIG-1), and at least a subset of local immune cells have an increased expression of a type 1 interferon in response to innate immune stimulation induced by a nucleic acid. In certain embodiments, provided compositions are characterized in that a nucleic acid is released from the polymeric biomaterial and is taken up by local cells so that at least a subset of local immune cells respond to a nucleic acid by modulating their immune response (e.g., activation of RIG-1), and at least a subset of local immune cells have an increased expression of a type 1 interferon in response to innate immune stimulation induced by a nucleic acid, and/or at least a subset of local immune cells have a change in level and/or activity of an immunomodulatory polypeptide.

In certain embodiments, provided compositions comprising at least one nucleic acid are characterized in that the immune cells that take up the nucleic exhibits at least one of the following biological activities: expressing the immunomodulatory polypeptide encoded by a released nucleic acid, activating an immunomodulatory pathway in response to a released nucleic acid, exhibiting an increased expression of a type 1 interferon in response to innate immune stimulation induced by a released nucleic acid, and/or exhibiting a change in level and/or activity of the immunomodulatory polypeptide.

In certain embodiments, provided compositions comprising at least one nucleic acid are characterized in that the immune cells that take up a released nucleic acid comprise myeloid and/or plasmacytoid dendritic cells. In certain embodiments, the cells that take up a released nucleic acid comprise non-immune cells. In certain embodiments, the cells that take up a released nucleic acid comprise non-immune cells, wherein the non-immune cells comprise fibroblasts and/or endothelial cells.

The storage modulus in a viscoelastic material measures the stored energy of the elastic portion of the material. Storage modulus may be measured with a rheometer. In some embodiments, measurements provided herein were made at room temperature with TA Instruments AR-G2 Magnetic Bearing Rheometer. The storage modulus of the drug delivery compositions will vary based on the components of the composition.

Generally, the relationship between storage modulus and concentration of thiol-modified hyaluronic acid (e.g., GLYCOSIL®) and the thiol-reactive PEGDA cross-linker (e.g., EXTRALINK®) is linear (excluding the limits of sensitivity). For example, a formulation of 0.8% GLYCOSIL® and 0.2% EXTRALINK® will have a storage modulus of about 100 Pa, and a formulation of 1.3% GLYCOSIL® and 2% EXTRALINK® will have a storage modulus of about 1600 Pa. In certain embodiments, provided compositions are liquid or a viscous polymer solution. In certain embodiments, provided compositions are polymer networks, or gels.

In certain embodiments, provided compositions have a storage modulus of less than 50 Pa, at least 50 Pa, at least 100 Pa, at least 200 Pa, at least 300 Pa, at least 400 Pa, at least 500 Pa, at least 600 Pa, at least 700 Pa, at least 800 Pa, at least 900 Pa, at least 1000 Pa, at least 1100 Pa, at least 1200 Pa, at least 1300 Pa, at least 1400 Pa, at least 1500 Pa, at least 1600 Pa, at least 1700 Pa, at least 1800 Pa, at least 1900 Pa, at least 2000 Pa, at least 2100 Pa, at least 2200 Pa, at least 2300 Pa, at least 2400 Pa, at least 2500 Pa, at least 2600 Pa, at least 2700 Pa, at least 2800 Pa, at least 2900 Pa, or at least 3000 Pa. In certain embodiments, provided compositions have a storage modulus of about 10 Pa to about 500 Pa.

In certain embodiments, provided compositions have a storage modulus of about 50 Pa to about 100,000,000 Pa, about 50 Pa to about 100,000 Pa, about 50 Pa to about 10,000 Pa, about 50 Pa to about 3,000 Pa, about 100 Pa to about 3,000 Pa, about 100 Pa to about 2,000 Pa, about 500 Pa to about 3,000 Pa, about 500 Pa to about 2,000 Pa, about 1,000 Pa to about 2,000 Pa, about 1,200 Pa to about 1,800 Pa, about 1,300 Pa to about 1,700 Pa, or about 1,400 Pa to about 1,600 Pa.

In certain embodiments, provided compositions have a storage modulus of up to about 600 Pa, up to about 700 Pa, up to about 800 Pa, up to about 900 Pa, up to about 1,000 Pa, up to about 1,100 Pa, up to about 1,200 Pa, up to about 1,300 Pa, up to about 1,400 Pa, up to about 1,500 Pa, up to about 1,600 Pa, up to about 1,700 Pa, up to about 1,800 Pa, up to about 1,900 Pa, up to about 2,000 Pa, up to about 2,500 Pa, up to about 3,000 Pa, up to about 5,000 Pa, up to about 10,000 Pa, up to about 100,000 Pa, up to about 1,000,000 Pa, up to about 10,000,000 Pa, or up to about 100,000,000 Pa.

In certain embodiments, provided compositions comprise at least one active/therapeutic agent (e.g., nucleic acid) and at least one polymeric biomaterial that forms a polymer network which acts as a matrix or depot, wherein at least some of the active agent(s) (e.g., nucleic acid acting as or encoding an activator or modulator of the innate immune system, or activator or modulator of the adaptive immune system) is dispersed within the polymer network. In some embodiments, provided compositions facilitate the release of one or more therapeutic agents under physiological conditions, such as within the body. In some embodiments, release of one or more therapeutic agents may occur at varying rates, depending on the components of the composition (e.g., identity and concentration of the polymer network). For example, in some embodiments, the release rate of one or more therapeutic agents (the time at which the therapeutic agent(s) is/are no longer a part of the composition or device) may be on the order of minutes, hours, days, weeks, months, or years. In some embodiments, therapeutic agents may be released by various mechanisms, e.g., by diffusion, chemical activity, enzymatic activity, or cellular machinery. In some embodiments, compositions described herein are stable in vivo such that they deliver drug to an intended target in a suitable amount of time.

In certain embodiments, compositions comprising at least one polymeric biomaterial and at least one active agent (e.g., nucleic acid) are characterized in that when tested in vitro by placing a combination of a polymeric biomaterial and an active agent in buffer (e.g., PBS pH 7.4), less than 100% of an active agent is released within 3 hours from the polymeric biomaterial. In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of an active agent is released in vitro within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after formation and placing into PBS (pH 7.4) of the composition or device.

In certain embodiments, compositions comprising at least one polymeric biomaterial and at least one active agent (e.g., nucleic acid) are characterized in that when tested in vivo by administering a provided composition to a mammary fat pad of a mouse subject, the composition extends release of the active agent such that, when assessed at 24 hours after administration, more active agent is present at the mammary fat pad than is observed when the active agent is administered in solution. In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of the active agent is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection within the mammary fat pad of a subject mouse) of the composition.

In certain embodiments, compositions comprising at least one polymeric biomaterial and at least one nucleic acid are characterized in that when tested in vivo by administering a provided composition to a mammary fat pad of a mouse subject, the composition extends release of a nucleic acid such that, when assessed at 24 hours after administration, more nucleic acid is present at the mammary fat pad than is observed when the nucleic acid is administered in solution. In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of a nucleic acid is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection within the mammary fat pad of a subject mouse) of the composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of an activator of the innate immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of an activator of the innate immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of a nucleic acid encoded activator of the innate immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of a nucleic acid encoded activator of the innate immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of an activator of the the adaptive immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of an activator of the adaptive immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of a nucleic acid encoded activator of the adaptive immune system is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of a nucleic acid encoded activator of the adaptive immune system is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of a nucleic acid is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of a nucleic acid is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 1% of the nucleic acid based modulator of the inflammatory innate immune response (e.g., nucleic acid acting as or encoding an inhibitor of proinflammatory immune response mediated by p38 mitogen-activated protein kinase (MAPK) pathway, or an activator of innate immunity which is or comprises a stimulator of interferon genes (STING) agonist or comprises a Toll-like receptor (TLR) 7 and/or TLR8 (“TLR7/8”) agonist) is released in vivo within 4 weeks, 3 weeks, 2 weeks, 10 days, 7 days, 6 days, 5 days, 4 days, 3 days, 2 days, 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, greater than or equal to 99%, greater than or equal to 95%, greater than or equal to 90%, greater than or equal to 80%, greater than or equal to 70%, greater than or equal to 60%, greater than or equal to 50%, greater than or equal to 40%, greater than or equal to 30%, greater than or equal to 20%, greater than or equal to 10%, greater than or equal to 5%, or greater than or equal to 1% of the nucleic acid based modulator of the inflammatory innate immune response (e.g., a nucleic acid acting as or encoding an inhibitor of proinflammatory immune response mediated by p38 mitogen-activated protein kinase (MAPK) pathway, or an activator of innate immunity which is or comprises a stimulator of interferon genes (STING) agonist or comprises a Toll-like receptor (TLR) 7 and/or TLR8 (“TLR7/8”) agonist) is released in vivo within 1 day, 18 hours, 12 hours, 8 hours, 6 hours, 4 hours, 3 hours, 2 hours, 1 hours, 45 minutes, 30 minutes, 20 minutes, 15 minutes, or 10 minutes after administration (e.g., implantation or injection) of the composition.

In certain embodiments, polymeric biomaterials useful for provided compositions described herein are biocompatible. In some embodiments, polymeric biomaterials are biodegradable. In some embodiments, provided compositions are able to be degraded, chemically and/or biologically, within a physiological environment, such as within the body. In some embodiments, degradation of a provided compositions may occur at varying rates, depending on the components and polymer network or hydrogel used. For example, in some embodiments, the half-life of a composition (the time at which 50% of the composition is degraded into monomers and/or other non-polymeric moieties) may be on the order of days, weeks, months, or years. In some embodiments, provided compositions may be biologically degraded, e.g., by enzymatic activity or cellular machinery, in some cases, for example, through exposure to a lysozyme (e.g., having relatively low pH), or by simple hydrolysis. In some embodiments, provided compositions may be broken down into monomers and/or other non-polymeric moieties that cells can either reuse or dispose of without significant toxic effect on the cells. In certain embodiments, provided compositions are stable in vivo such that they deliver drug to the intended target in a suitable amount of time.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 12 months after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 6 months after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 5 months after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 4 months after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 3 months after administration (e.g., implantation or injection) a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 2 months after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 1 month after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 1 week after administration (e.g., implantation or injection) of a provided composition.

In certain embodiments, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 4%, less than or equal to 3%, less than or equal to 2%, less than or equal to 1%, less than or equal to 0.5%, or less than or equal to 0.1%, of a composition remains in vivo 1 day after administration (e.g., implantation or injection) of a provided composition.

### Preparation and Administration of Provided Compositions

In certain embodiments, the present disclosure provides compositions comprising therapeutic agents (e.g., nucleic acids), as described herein. In certain embodiments, the therapeutic agents are provided in an effective amount in the drug delivery compositions to treat and/or prevent a disease (e.g., a proliferative disease, such as cancer). In certain embodiments, the effective amount is a therapeutically effective amount of a particular therapeutic agent. In certain embodiments, the effective amount is a prophylactically effective amount of a particular therapeutic agent.

Provided compositions described herein can be prepared by any method known in the art of pharmacology. In certain embodiments, such preparatory methods comprise the steps of adding a polymeric biomaterial (e.g., thiol-modified hyaluronic acid) into a mold; adding a therapeutic agent (e.g., a nucleic acid acting as or encoding a modulator of the immune system); optionally adding an activator of adaptive immune response to the mold; optionally adding a chemokine or cytokine to the mold; optionally adding an activator of innate immune response to the mold; wherein any of the optional agents may also comprise a nucleic acid, adding a cross-linking agent to the mold (e.g., a thiol-reactive PEGDA cross-linker); and allowing the composition to stand for at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 90 minutes, at least 2 hours, at least 3 hours, at least 4 hours, at least 5 hours, or at least 6 hours for solidification.

In certain embodiments, provided compositions comprise a viscous solution of at least one nucleic acid and at least one non-positively charged polymeric biomaterial. In certain embodiments, the non-positively charged polymeric biomaterial is hyaluronic acid. In certain embodiments, the non-positively charged polymeric biomaterial is a neutral polymer (e.g., methylcellulose, PEG, etc).

In certain embodiments, provided compositions comprise a solution which undergoes ionic crosslininking between at least one nucleic acid, and at least one positively charged polymeric biomaterial. In certain embodiments, provided compositions are pre-mixed in preparation for injection, or are injected using a dual-barrel syringe system, wherein crosslinking occurs in-situ. In certain embodiments, the positively charged polymer is chitosan. In certain embodiments, provided compositions are mixed at the time of injection and undergo ionic crosslinking in situ, wherein compositions comprise at least one positively charged polymeric biomaterial (e.g., chitosan or alginate), at least one nucleic acid, and at least one crosslinking facilitator (e.g., tripolyphosphate, or Ca²⁺). In certain embodiments, at least one nucleic acid can be mixed in solution with either the crosslinking agent, or with the polymeric biomaterial prior to injection at a target site.

In certain embodiments, provided compositions comprising at least one polymeric biomaterial (e.g., poloxamer) and at least one nucleic acid undergo thermal induced crosslinking in situ post-injection at a target site.

In certain embodiments, the polymeric biomaterial comprises thiol-modified hyaluronic acid (e.g., GLYCOSIL®). In certain embodiments, thiol-modified hyaluronic acid is included at a concentration suitable for the preparation of the polymer network or hydrogel, and is, by weight/volume, about 1% to about 10%, about 1% to about 5%, about 1% to about 3%, or about 1.5% to about 2.5%; and the amount of thiol-reactive PEGDA cross-linker (e.g., EXTRALINK®) used for the preparation of the polymer network or hydrogel is, by weight/volume, about 0.5% to about 2%, about 1% to about 3%, is about 1% to about 20%, about 10% to about 20%, about 5% to about 15%, or about 10% to about 15%. In certain embodiments, the concentration of thiol-modified hyaluronic acid is about 2% w/v and the concentration of thiol-reactive PEGDA cross-linker is about 12.5% w/v. In certain embodiments, the formulation of 2% thiol-modified hyaluronic acid and 12.5% provides a polymer network or hydrogel with a storage modulus of about 1000 Pa to about 2000 Pa. In certain embodiments, the concentration of thiol-modified hyaluronic acid is about 1% w/v and the concentration of thiol-reactive PEGDA cross-linker is about 0.5% w/v.

For the preparation of standard tissue engineering applications known in the art, the typical concentration of thiol-modified hyaluronic acid (e.g., GLYCOSIL®) is about 1% w/v and the typical concentration of thiol-reactive PEGDA cross-linker (e.g., EXTRALINK®) is about 1% w/v. Thus, the use of 2% w/v thiol-modified hyaluronic acid (e.g., GLYCOSIL®) and 12.5% w/v thiol-reactive PEGDA cross-linker (e.g., EXTRALINK®) provides an unexpectedly useful and advantageous biomaterial in the disclosed drug delivery compositions.

In certain embodiments, the concentration of the alginate used for the preparation of the polymer network or hydrogel is, by weight/volume, about 0.5% to about 2.5%, about 0.75% to about 2.0%, or about 1.0% to about 1.5% alginate. In certain embodiments, the amount of 1 M calcium chloride cross-linker solution used for the preparation of the polymer network or hydrogel is about 5 μL to 25 μL, about 10 μL to 20 μL, or about 15 μL. In certain embodiments, the payload of interest can be loaded in about 10 μL to 70 μL solvent (PBS or DMSO), 20 μL to 60 μL solvent (PBS or DMSO), about 30 μL to 50 μL solvent (PBS or DMSO), or about 40 μL solvent (PBS or DMSO).

In some embodiments, provided compositions may further comprise at least one excipient. In certain embodiments, the excipient is phosphate-buffered saline, tris(hydroxymethyl)aminomethane, sodium chloride, potassium chloride, calcium chloride, magnesium sulfate, sodium bicarbonate, sodium phosphate, potassium phosphate, calcium nitrate, glucose, lactose, trehalose, sucrose, or a combination thereof. In certain embodiments, the excipient is phosphate-buffered saline, tris(hydroxymethyl)aminomethane, sodium chloride, or a combination thereof. In certain embodiments, the excipient is phosphate-buffered saline.

In certain embodiments, provided compositions do not include nanoparticles or microparticles. In certain embodiments, the drug delivery compositions do not include silica microparticles, polyethylene microparticles, polystyrene microparticles, polyester microparticles, polyanhydride microparticles, polycaprolactone microparticles, polycarbonate microparticles, or polyhydroxybutyrate microparticles. In certain embodiments, provided compositions do not include porous silica microparticles.

In certain embodiments, provided compositions include one or more organic solvents. In certain embodiments, compositions include dimethylsulfoxide (DMSO).

In certain embodiments, provided compositions do not include organic solvent. In certain embodiments, organic solvents are not used in the preparation of the compositions. In certain embodiments, provided compositions are free of organic solvent. In certain embodiments, compositions are substantially free of organic solvent. In certain embodiments, compositions comprise, by weight, less than 10%, less than 5%, less than 4%, less than 3%, less than 2%, less than 1%, less than 0.5%, less than 0.1%, less than 0.01%, less than 0.001%, or less than 0.0001% of organic solvent. In certain embodiments, compositions comprise, by weight, less than 1000 ppm, less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 1 ppm, less than 10 ppb, or less than 1 ppb of organic solvent. In certain embodiments, provided compositions do not include dimethylsulfoxide (DMSO).

In certain embodiments, provided compositions comprise organic solvent(s). In certain embodiments, an organic solvent is cyclodextrin, methanol, ethanol, isopropanol, ethylene glycol, propylene glycol, and/or a combination thereof.

In certain embodiments, provided compositions can be prepared, packaged, and/or sold in bulk, as a single unit dose, and/or as a plurality of single unit doses. A “unit dose” is a discrete amount of the composition comprising a predetermined amount of the therapeutic agents and/or a predetermined volume of composition. The amount of the therapeutic agents is generally equal to the dosage of the therapeutic agents which would be administered to a subject and/or a convenient fraction of such a dosage, such as, for example, one-half, one-third, or one-quarter of such a dosage.

In some embodiments, relative amounts of polymeric biomaterial(s), therapeutic agent(s), excipient(s), and/or any additional ingredients in a composition of the disclosure will vary, depending upon the identity, size, and/or condition of the subject treated. By way of example, the composition may comprise between 0.1% and 99% (w/w), between 0.1% and 90% (w/w), between 0.1% and 80% (w/w), between 0.1% and 70% (w/w), between 1% and 50% (w/w), between 10% and 80% (w/w), between 10% and 90% (w/w), between 10% and 80% (w/w), between 20% and 80% (w/w), between 30% and 80% (w/w), between 30% and 70% (w/w), or between 40% and 60% (w/w), of at least one therapeutic agent.

In some embodiments, additional pharmaceutically acceptable excipients may be used in the manufacture of provided compositions. For example, in some embodiments, these comprise inert diluents, dispersing and/or granulating agents, surface-active agents and/or emulsifiers, disintegrating agents, binding agents, preservatives, buffering agents, lubricating agents, and/or oils. Excipients such as cocoa butter and suppository waxes, coloring agents, and/or coating agents may also be present in provided compositions.

Exemplary diluents comprise calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or mixtures thereof.

Exemplary granulating and/or dispersing agents comprise potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and/or mixtures thereof.

Exemplary surface active agents and/or emulsifiers comprise natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80)), polyoxyethylene esters (e.g., polyoxyethylene monostearate (MYRJ 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (BRIJ 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC F-68, Poloxamer-188, cetrimonium bromide, cetylpyridinium chloride, benzalkonium chloride, docusate sodium, and/or mixtures thereof.

Exemplary binding agents comprise starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM), and larch arabogalactan), alginates, polyethylene oxide, polyethylene glycol, inorganic calcium salts, silicic acid, polymethacrylates, waxes, water, alcohol, and/or mixtures thereof.

Exemplary preservatives comprise antioxidants, chelating agents, antimicrobial preservatives, antifungal preservatives, alcohol preservatives, acidic preservatives, and/or other preservatives. In certain embodiments, the preservative is an antioxidant. In other embodiments, the preservative is a chelating agent.

Exemplary antioxidants comprise alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite.

Exemplary chelating agents comprise ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and/or any combination thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal.

Exemplary antifungal preservatives comprise butyl paraben, methyl paraben, ethyl paraben, propyl paraben, benzoic acid, hydroxybenzoic acid, potassium benzoate, potassium sorbate, sodium benzoate, sodium propionate, and/or sorbic acid.

Exemplary alcohol preservatives comprise ethanol, polyethylene glycol, phenol, phenolic compounds, bisphenol, chlorobutanol, hydroxybenzoate, and/or phenylethyl alcohol.

Exemplary acidic preservatives comprise vitamin A, vitamin C, vitamin E, beta-carotene, citric acid, acetic acid, dehydroacetic acid, ascorbic acid, sorbic acid, and/or phytic acid.

Other preservatives comprise tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS, PHENONIP, methylparaben, GERMALL 115, GERMABEN II, NEOLONE, KATHON, and/or EUXYL.

Exemplary buffering agents comprise citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and/or mixtures thereof.

Exemplary lubricating agents comprise magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and/or mixtures thereof.

Exemplary natural oils comprise almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or mixtures thereof.

Although the descriptions of compositions provided herein are principally directed to compositions which are suitable for administration to humans, it will be understood by the skilled artisan that such compositions are generally suitable for administration to animals of all sorts. In some embodiments, provided compositions suitable for administration to humans may be modified in order to render the compositions suitable for administration to various animals, the methodology for this process is well understood, and the ordinarily skilled veterinary pharmacologist can design and/or perform such modification with ordinary experimentation.

In some embodiments, provided compositions provided herein are typically formulated in a size (e.g., volume) and weight appropriate for the intended use (e.g., surgical implantation or injection) for ease of administration. It will be understood, however, that the total amount of the composition of the present disclosure (e.g., number or volume of composition implanted or injected) will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; the drugs used in combination or coincidental with the specific active ingredient employed; and like factors well known in the medical arts.

In some embodiments, provided compositions can be administered by surgical implantation and/or injection. For example, in some embodiments, provided compositions may be administered by surgical implantation in the void volume of a resected tumor at the time of tumor resection surgery. In some embodiments, surgical implantation in the void volume of a resected tumor occurs at a time after the primary tumor resection procedure. In some embodiments, provided compositions may be administered by injection into the void volume of a resected tumor at the time of tumor resection. In some embodiments, provided compositions may be administered by injection to the void volume of a resected tumor after tumor resection. In some embodiments, provided compositions are injected to a target site (e.g., site of tumor resection) less than 1 day, less than 2 days, less than 3 days, less than 4 days, less than 5 days, less than 6 days, or less than 7 days after tumor resection. In some embodiments, provided compositions are injected to a target site (e.g., site of tumor resection) less than 1 week, less than 2 weeks, less than 3 weeks, less than 4 weeks, less than 1 month, less than 3 months, less than 6 months, or less than 12 months after tumor resection.

In some embodiments, provided compositions may be administered by surgical implantation and affixed with a bioadhesive. In certain embodiments, compositions are affixed with a bioadhesive in the void volume of a resected tumor.

In certain embodiments, provided compositions are administered by surgical implantation or injection at a site within 100 cm, 90 cm, 80 cm. 70 cm, 60 cm. 50 cm, 40 cm, 30 cm, 20 cm, 10 cm, 9 cm, 8 cm, 7 cm, 6 cm, 5 cm, 4 cm, 3 cm, 2 cm, 1 cm, 9 mm, 8 mm, 7 mm, 6 mm, 5 mm, 4 mm, 3 mm, 2 mm, or 1 mm of the void volume of a resected tumor. In certain embodiments, the void volume of a resected tumor is the void volume of a resected organ having a tumor (e.g., lung, kidney, pancreas, liver, colon, testes, ovary, breast, appendix, bladder). In certain embodiments, the void volume of a resected tumor is the void volume of a resected portion of an organ having a tumor (e.g., lung, kidney, pancreas, liver, colon, testes, ovary, breast, appendix, bladder).

In certain embodiments, precursor composition components e.g., at least one polymeric biomaterial, at least one nucleic acid, and optional cross-linking agent(s) are administered separately to a subject (e.g., at the site of tumor resection), wherein the precursor components form a composition in vivo . . . precursor composition components e.g., at least one polymeric biomaterial, at least one nucleic acid, and optional cross-linking agent(s) are administered sequentially. In certain embodiments, precursor composition components e.g., at least one polymeric biomaterial, at least one nucleic acid, and optional cross-linking agent(s) are administered concurrently. precursor composition components e.g., at least one polymeric biomaterial, at least one nucleic acid, and optional cross-linking agent(s) are administered as a mixture. In certain embodiments, the administration is via injection.

The exact amount of the therapeutic agents required to achieve effective amounts will vary from subject to subject, depending, for example, on species, age, and general condition of a subject, severity of the side effects or disorder, identity of the particular agent(s), and the like. In certain embodiments, an effective amount of provided compositions for

administration to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg.

In certain embodiments, provided compositions may be at dosage levels sufficient to deliver about 0.001 mg/kg to about 100 mg/kg, about 0.01 mg/kg to about 50 mg/kg, about 0.1 mg/kg to about 40 mg/kg, about 0.5 mg/kg to about 30 mg/kg, about 0.01 mg/kg to about 10 mg/kg, about 0.1 mg/kg to about 10 mg/kg, or about 1 mg/kg to about 25 mg/kg, of subject body weight per day, of any of the therapeutic agents present in the composition, to obtain the desired therapeutic effect.

It will be appreciated that dose ranges as described herein provide guidance for the administration of the provided compositions to an adult. The amount to be administered to, for example, a child or an adolescent can be determined by a medical practitioner or person skilled in the art and can be lower or the same as that administered to an adult.

It will be also appreciated that compositions, as described herein, in some embodiments, can be administered in combination with one or more additional pharmaceutical agents. For example, in some embodiments, provided compositions can be administered in combination with additional pharmaceutical agents that reduce and/or modify their metabolism, inhibit their excretion, and/or modify their distribution within the body. It will also be appreciated that the additional therapy employed in some embodiments may achieve a desired effect for the same disorder, and/or it may achieve different effects.

In some embodiments, provided compositions can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents will be administered separately in different doses and/or different routes of administration. The particular combination to employ in a regimen will take into account compatibility of provided composition with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-inflammatory agents, immunosuppressant agents, and pain-relieving agents. Pharmaceutical agents include small molecule therapeutics such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and/or cells.

In certain embodiments, provided compositions do not include cells. In certain embodiments, compositions do not include adoptively transferred cells. In certain embodiments, compositions do not include T cells. In certain embodiments, the additional pharmaceutical agent is not adoptively transferred cells. In certain embodiments, the additional pharmaceutical agent is not T cells. In certain embodiments, compositions do not include tumor antigens. In certain embodiments, compositions do not include tumor antigens loaded ex vivo.

In certain embodiments, provided compositions are prepared just prior to in vivo implantation (e.g., in an operating room or close proximity). In certain embodiments, provided compositions are prepared within 24 hours, 18 hours, 12 hours, 11 hours, 10 hours, 9 hours, 8 hours, 7 hours, 6 hours, 5 hours, 4 hours, 3 hours, 2 hours, 1 hour, 30 minutes, 20 minutes, 10 minutes, 5 minutes, or 1 minute of in vivo implantation.

In certain embodiments, provided compositions are prepared in advance of in vivo implantation. In certain embodiments, compositions are prepared within 31 days, 28 days, 21 days, 14 days, 7 days, 6 days, 5 days, 4 days, 3 days, 2 days, or 1 day of in vivo implantation.

In certain embodiments, provided compositions are prepared within 1 year, 10 months, 8 months, 6 months, 4 months, 3 months, 2 months, 31 days, 28 days, 21 days, 14 days, 7 days, 6 days, 5 days, 4 days, 3 days, 2 days, or 1 day of its use in a therapeutic setting.

Also encompassed by the disclosure are kits. The kits provided may comprise compositions described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the kit comprises composition precursor components (e.g., polymeric biomaterial(s) and nucleic acid(s).

In certain embodiments, provided kits comprise a composition comprised of at least one polymeric biomaterial, and at least one nucleic acid. In certain embodiments, provided kits comprise a composition comprised of at least one polymeric biomaterial, at least one nucleic acid, and at least one polynucleotide carrier agent. In certain embodiments, provided kits comprise a composition comprised of at least one polymeric biomaterial, at least one nucleic acid. At least one polynucleotide carrier agent, and at least one additional immunomodulatory component. In certain embodiments, provided kits comprise a composition comprised of polymeric biomaterial(s) and an activator of a pro-inflammatory pathway (e.g., a RIG-1 agonist or nucleic acid encoding a modulator of the immune system). In certain embodiments, provided kits comprise a composition comprised of a polymeric biomaterial(s), an activator of a pro-inflammatory pathway (e.g., a RIG-1 agonist or nucleic acid encoding a modulator of the immune system), and a polynucleotide agent carrier. In certain embodiments, provided kits comprise a composition comprised of polymeric biomaterial(s), an inhibitor of a pro-inflammatory pathway (e.g., an inhibitor of proinflammatory immune response mediated by p38 MAPK pathway), and an activator of innate immune response. In certain embodiments, provided kits comprise a composition comprised of polymeric biomaterial(s), an inhibitor of a pro-inflammatory pathway (e.g., an inhibitor of proinflammatory immune response mediated by p38 MAPK pathway), and a cytokine. In certain embodiments, provided kits comprise a composition comprised of polymeric biomaterial(s), an inhibitor of a pro-inflammatory pathway (e.g., an inhibitor of proinflammatory immune response mediated by p38 MAPK pathway), and an activator of adaptive immune response. In certain embodiments, the kit further comprises an activator of innate immune function. In certain embodiments, the kit further comprises a cytokine. In certain embodiments, the kit further comprises a nucleic acid carrier. In certain embodiments, the kit further comprises an activator of adaptive immune response. In certain embodiments, the kit further comprises a modulator of macrophage effector function. In certain embodiments, the kit further comprises an additional activator of adaptive immune response. In certain embodiments, the kit further comprises an oncolytic virus, a radioactive isotope, an immunomodulatory chemotherapeutic agent, a targeted agent, or a combination thereof. In certain embodiments, the kit comprises any composition described herein.

In certain embodiments, the kit does not comprise a chemotherapeutic agent. In certain embodiments, the kit does not comprise a cytotoxic agent.

In certain embodiments, a kit described herein further comprises instructions for using the kit. A kit described herein may also comprise information as required by a regulatory agency such as the U.S. Food and Drug Administration (FDA). In certain embodiments, the information comprised in the kits is prescribing information. In certain embodiments, the kits and instructions provide for treating cancer. In certain embodiments, kits described herein may comprise one or more additional pharmaceutical agents described herein as a separate composition.

### Methods of Treatment and Uses

The present disclosure provides methods of using compositions described herein, for the treatment and/or prevention of a proliferative disease, such as cancer (e.g., a sarcoma, a carcinoma, a lymphoma, a germ cell tumor, or a blastoma), in a subject. In some embodiments, compositions described herein are for use in treatment of a tumor which is amenable to resection. In some embodiments, compositions described herein are for use in treatment of lymphoma present in a spleen or a tissue outside of a lymphatic system, e.g., a thyroid or stomach.

In some embodiments, compositions described herein are useful in treating cancer, including a metastatic cancer. In some embodiments, provided compositions are useful to delay the onset of, slow the progression of, or ameliorate the symptoms of cancer. In some embodiments, provided compositions are useful to prevent cancer, and/or to prevent primary tumor regrowth. In some embodiments, provided compositions are useful to prevent tumor metastasis. In some embodiments, compositions described herein are administered in combination with other compounds, drugs, or therapeutic agents to treat cancer.

In certain embodiments, the cancer is a solid tumor. In some embodiments, the cancer is a solid tumor that may also be shedding metastatic cancerous cells. In certain embodiments, the cancer is a sarcoma, a carcinoma, a lymphoma, a germ cell tumor, a blastoma, or a combination thereof. In certain embodiments, the tumor is a sarcoma, a carcinoma, a lymphoma, a germ cell tumor, a blastoma, or a combination thereof.

In some embodiments, compositions described herein are useful for treating a cancer, including metastatic cancers. In some embodiments, compositions described herein are useful for treating cancers including, but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); parathryroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In certain embodiments, the cancer is breast cancer. In certain embodiments, the cancer is skin cancer. In certain embodiments, the cancer is melanoma. In certain embodiments, the cancer is lung cancer. In certain embodiments, the cancer is kidney cancer. In certain embodiments, the cancer is liver cancer. In certain embodiments, the cancer is pancreatic cancer. In certain embodiments, the cancer is colorectal cancer. In certain embodiments, the cancer is bladder cancer. In certain embodiments, the cancer is lymphoma. In certain embodiments, the cancer is prostate cancer. In certain embodiments, the cancer is thyroid cancer.

In some embodiments, compositions described herein are useful in treating adenocarcinoma, adrenal gland cancer, anal cancer, angiosarcoma, appendix cancer, bile duct cancer, bladder cancer, bone cancer, brain cancer, breast cancer, bronchus cancer, carcinoid tumor, cardiac tumor, cervical cancer, choriocarcinoma, chordoma, colorectal cancer, connective tissue cancer, craniopharyngioma, ductal carcinoma in situ, endotheliosarcoma, endometrial cancer, ependymoma, epithelial carcinoma, esophageal cancer, Ewing's sarcoma, eye cancer, familiar hypereosinophilia, gall bladder cancer, gastric cancer, gastrointestinal carcinoid tumor, gastrointestinal stromal tumor (GIST), germ cell cancer, head and neck cancer, hemangioblastoma, histiocytosis, Hodgkin lymphoma, hypopharynx cancer, inflammatory myofibroblastic tumors, intraepithelial neoplasms, immunocytic amyloidosis, Kaposi sarcoma, kidney cancer, liver cancer, lung cancer, leiomyosarcoma (LMS), melanoma, midline tract carcinoma, multiple endocrine neoplasia syndrome, muscle cancer, mesothelioma, myeloproliferative disorder (MPD), nasopharynx cancer, neuroblastoma, neurofibroma, neuroendocrine cancer, non-Hodgkin lymphoma, osteosarcoma, ovarian cancer, pancreatic cancer, paraneoplastic syndromes, parathryroid cancer, papillary adenocarcinoma, penile cancer, pharyngeal cancer, pheochromocytoma, pinealoma, pituitary cancer, pleuropulmonary blastoma, primitive neuroectodermal tumor (PNT), plasma cell neoplasia, prostate cancer, rectal cancer, retinoblastoma, rhabdomyosarcoma, salivary gland cancer, sebaceous gland carcinoma, skin cancer, small bowel cancer, small intestine cancer, soft tissue sarcoma, stomach cancer, sweat gland carcinoma, synovioma, testicular cancer, thymic cancer, thyroid cancer, urethral cancer, uterine cancer, vaginal cancer, vascular cancer, vulvar cancer, or a combination thereof.

In some embodiments, compositions described herein are useful in treating and/or preventing solid tumors and metastases.

In some embodiments, compositions described herein, are administered to a target. In some embodiments, a target comprises a site in a subject who has recently undergone a tumor resection. In some embodiments, the tumor resection subject is suffering from metastatic cancer. In some embodiments, the tumor resection site is characterized by absence of gross residual tumor antigen. In some embodiments, the administration target site is the tumor resection site. In some embodiments, the target site is a site near the tumor resection site. In some embodiments, a site near the tumor resection site is within 4 inches of the tumor resection site. In some embodiments, the target site is a sentinel lymph node. In some embodiments, the target site is near a sentinel lymph node. In some embodiments, the target site is a draining lymph node. In some embodiments, a target site is a site at which cancer cells have been treated or killed by prior cancer therapy, e.g., chemotherapy or radiation. In some embodiments, further treatment includes comprising a step of monitoring at least one metastatic site in the tumor resection subject after the administration.

In some embodiments, provided compositions administered to a target site are administered as a pre-formed gel or semisolid polymer network, and the compositions can be administered to the target site by implantation. In some embodiments, provided compositions administered to a target site is in an injectable format (e.g., a liquid or a colloidal solution). In some embodiments, administration as described herein involves administration of one or more polymeric biomaterial precursor components that interact or react in situ to form a gel or colloidal solution composition as described herein; in some such embodiments, such interaction or reaction involves crosslinking which may, in some embodiments, occur spontaneously and in some embodiments may be triggered by application of an agent (e.g., a catalyst and/or a reactant) and/or a condition (e.g., one or more of heat, pH, pressure, electromagnetic radiation which may be at a particular wavelength, etc).

In certain embodiments, the methods described herein include implanting (e.g., via administration of a polymeric biomaterial network or a set of precursors thereof as described herein) in a subject an effective amount of compositions described herein. In certain embodiments, the methods described herein include surgically implanting in a subject an effective amount of compositions described herein. In certain embodiments, the methods described herein further comprise implanting provided compositions after surgical resection of a tumor. In certain embodiments, the methods described herein further comprise implanting a provided composition at the site of tumor resection. In certain embodiments, the methods described herein further comprise implanting a provided composition in the void volume of the resected tumor. In certain embodiments, the methods described herein further comprise implanting a provided composition in the tumor resection site during tumor resection surgery.

In certain embodiments, the methods described herein comprise administering (e.g., implanting or injecting) provided compositions after removal of, by weight, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99% of the resected tumor. In certain embodiments, the methods described herein comprise administering (e.g., implanting or injecting) provided compositions after removal of, by volume, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99% of the resected tumor.

In certain embodiments, the methods described herein do not comprise administering (e.g., implanting or injecting) a provided composition to a site adjacent to a tumor. In certain embodiments, the methods described herein do not comprise administering (e.g., implanting or injecting) a provided composition adjacent to a tumor without resection of the tumor.

In certain embodiments, compositions described herein are administered in combination with one or more additional therapeutic agents as described herein. In certain embodiments, the additional therapeutic agent is an anti-cancer agent.

In certain embodiments, compositions described herein do not include, and the steps of administration do not involve, administration of a tumor antigen to the tumor resection subject. One skilled in the art understands that tumor antigens include but are not limited to: Mart-1, gp100, CEA, CD19, NY-ES01, MAGE-A3, hTERT, EGFR, mesothelin, HPV, EBV, MCC, Mum-1, beta-catenin, CDK4, and ERBB2IP, see e.g. Ilyas & Yang, “Landscape of tumor antigens in T-cell immunotherapy” J Immunol. (2015) 195 (11): 5117-5122, the contents of which are incorporated herein by reference in their entirety for the purposes described herein. In certain embodiments, provided compositions do not include, and the steps of administration do not involve, the transfer of adoptive immune cells to the tumor resection subject. One skilled in the art understands that transfer of adoptive immune cells includes but is not limited to the transfer of: CD8+ T-cells, CD4+ T-cells, Dendritic cells, Natural Killer cells, Gamma Delta T cells, etc.

In certain embodiments, provided compositions do not include nanoparticles or microparticles. In certain embodiments, the administration step of the exemplary composition does not involve administration of a microparticle to the tumor resection subject. Nanoparticles include particles between 1 and 100 nm in size. Microparticles include particles between 0.1 and 100 μm in size. In certain embodiments, provided compositions do not include silica microparticles, polyethylene microparticles, polystyrene microparticles, polyester microparticles, polyanhydride microparticles, polycaprolactone microparticles, polycarbonate microparticles, or polyhydroxybutyrate microparticles. In certain embodiments, provided compositions do not include porous silica microparticles.

In certain embodiments, treatment with provided compositions comprises the further steps of determining (i) whether nucleic acid(s) released from the candidate polymeric biomaterial are taken up by non-immune cells; and/or (ii) whether the nucleic acid(s) exerts an adverse effect on the non-immune cells.

In certain embodiments, the subject being treated is a mammal. In certain embodiments, the subject is a human. In certain embodiments, the subject is a human patient who has received neoadjuvant (pre-operative) chemotherapy. In certain embodiments, the subject is a human patient who has received neoadjuvant radiation therapy. In certain embodiments, the subject is a human patient who has received neoadjuvant chemotherapy and radiation therapy. In certain embodiments, the subject is a human patient who has received neoadjuvant molecular targeted therapy. In certain embodiments, the subject is a human patient who has received neoadjuvant immunotherapy, including immune checkpoint blockade (e.g., anti-CTLA-4, anti-PD-1, and/or anti-PD-L1). In certain embodiments, the subject is a human patient who has not received neoadjuvant immunotherapy, including immune checkpoint blockade (e.g., anti-CTLA-4, anti-PD-1, and/or anti-PD-L1). In certain embodiments, the subject is a human patient whose tumor has not objectively responded to neoadjuvant therapy (as defined by Response Evaluation Criteria in Solid Tumors (RECIST) or immune-related Response Criteria (irRC)) (e.g., stable disease, progressive disease). In certain embodiments, the subject is a human patient whose target lesion has objectively responded to neoadjuvant therapy (e.g., partial response, complete response). Non-target lesions may exhibit an incomplete response, stable disease, or progressive disease. In certain embodiments, the subject is a human patient who would be eligible to receive immunotherapy as a standard of care in the adjuvant (post-operative) setting. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal, such as a rodent, pig, dog, or non-human primate. In certain embodiments, the subject is a non-human transgenic animal such as a transgenic mouse or transgenic pig.

### EXAMPLES

In order that the invention described herein may be more fully understood, the following examples are set forth. It should be understood that these examples are for illustrative purposes only and are not to be construed as limiting this invention in any manner.

### Example 1. Preparation of an Exemplary Nucleic Acid Delivery Compositions for Cancer Treatment

The present Example describes the creation of a sustained release composition comprising a nucleic acid immunomodulatory agents suitable for administration to a tumor resection site for cancer treatment. In some embodiments, such a nucleic acid immunomodulatory agent is or comprises an agonist of the innate immune system. In some embodiments, such a sustained release composition comprises at least one polymeric biomaterial and at least one nucleic acid, or at least one polymeric biomaterial, at least one nucleic acid and at least one nucleic acid carrier.

To prepare the compositions described within some embodiments herein, innate immune system agonists (e.g., nucleic acids), are complexed with a nucleic acid carrier (e.g., a transfection agent). In some embodiments, components are mixed in a volumetric ratio (e.g., a 1:1 ratio). In some embodiments the components are prepared in a sugar solution (e.g., a glucose solution). In some embodiments, the mixture is then incubated for a period of time.

To prepare the compositions described within some embodiments herein, innate immune system agonists (e.g., RIG-1 agonist, Invivogen #tlrl-hprna) are complexed with a nucleic acid carrier (e.g., In vivo-jetPEI, PolyPlus #201-10G). In some embodiments, components are mixed in a volumetric ratio (e.g., a 1:1 ratio). In some embodiments these in vivo-jetPEI/RIG-I agonist complexes are prepared by mixing 1:1 volume ratio of RIG-I agonist (Invivogen, #tlrl-hprna) in a 5% glucose solution with the in vivo-jetPEI reagent (N/P=6) in 5% glucose solution (PolyPlus, #201-10G). In some embodiments the mixture is then incubated at room temperature for an appropriate period of time (e.g., for approximately 15 minutes) and is then added to a polymeric biomaterial, or polymer network.

To prepare compositions according to some embodiments described herein, polymer networks were prepared using commercially available kits. In alternative embodiments described herein, polymer networks are prepared using unique polymer combinations not commercially available. In some embodiments, the polymeric biomaterial which has the potential to create a polymer network was placed within a mold. In some embodiments, the nucleic acid and nucleic acid carrier components were then added to the polymeric biomaterial. In some embodiments, chemical crosslinker was then added, and the product was allowed to incubate, creating a crosslinked polymer network. In some embodiments, chemical crosslinker is not required and gelation occurs during incubation under appropriate conditions (e.g., gelation enabling temperature) creating a suitable polymer network.

To prepare compositions according to some embodiments described herein, polymer networks were prepared using the Hystem hydrogel kits (ESI Bio, GS1004). First, 120 μL of Glycosil (a crosslinkable hyaluronic acid e.g., a thiol-modified hyaluronic acid) was added into a Teflon mold (9 mm diameter), and then In vivo-jetPEI/RIG-I agonist (2.5 μg, 7.5 μg, or 20 μg) was added, and 30 μL of chemical crosslinker (e.g., a thiol-reactive crosslinker such as polyethylene diacrylate, e.g., EXTRALINK® polyethylene glycol diacrylate cross-linker) was combined, the product was incubated for 1 hour to allow the polymer network to cross-link.

### Example 2. Use of Exemplary Nucleic Acid Delivery Compositions for In Vivo Cancer Treatment

The present Example describes administration of a sustained release composition comprising a nucleic acid immunomodulatory agent at a tumor resection site for cancer treatment. In some embodiments, such a nucleic acid immunomodulatory agent is or comprises an agonist of the innate immune system. In some embodiments, such a sustained release composition comprises at least one polymeric biomaterial and at least one nucleic acid, or at least one polymeric biomaterial, at least one nucleic acid and at least one nucleic acid carrier.

In some embodiments, in vivo studies utilized breast cancer cell lines grown in vitro. In some embodiments, prior to implantation, breast cancer cells were cultured in medium containing fetal bovine serum and an appropriate selective agent. In some embodiments, cells were cultured at physiological temperature, with known humidity, oxygen, and CO₂ concentrations.

In some embodiments, 4T1-Luc2 breast cancer cells were cultured in RPMI-1640 medium, with 10% fetal bovine serum (FBS) and 1% penicillin/streptomycin. In some embodiments, cells were cultured in a 37° C. humidified incubator, with 5% CO₂.

In some embodiments, in vivo animal studies are performed in mice. In some embodiments, mice are inoculated orthotopically with breast cancer cells in a mammary fat pad. In some embodiments, surgery is performed at an appropriate time following inoculation (e.g., at approximately day 10 after tumor inoculation). In some embodiments, concurrent to tumor excision, a composition as described herein comprising polymeric biomaterials or a polymer network loaded with an innate immune response agonist is administered at the tumor resection site. In some embodiments, a polymeric biomaterial or polymer network without an innate immune response agonist is administered at the tumor resection site to act as a relative negative control. In some embodiments, prolonged survival benefits are observed upon extended local release of an innate immune agonist.

In some embodiments, in vivo animal studies were performed in mice. In some embodiments, mice were inoculated orthotopically with breast cancer cells in a mammary fat pad. In some embodiments, surgery was performed at day 10 after tumor inoculation. In some embodiments, concurrent to tumor excision, a composition as described herein comprising polymeric biomaterials or a polymer network loaded with an innate immune response agonist was administered at the tumor resection site. In some embodiments, a polymeric biomaterial or polymer network without an innate immune response agonist was administered at the tumor resection site to act as a relative negative control. In some embodiments, prolonged survival benefits were observed upon extended local release of an innate immune agonist.

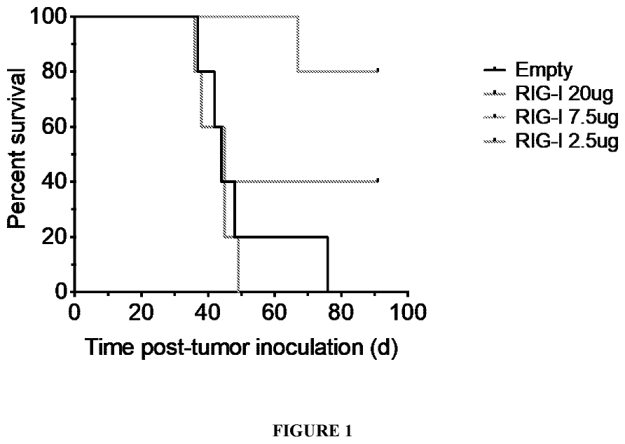

In one embodiment, in vivo animal studies were performed in 6-8 week old female BALB/cJ mice. Mice were inoculated orthotopically with 10⁵ 4T1-Luc2 breast cancer cells in their fourth mammary fat pad. Mice were size-matched and randomly assigned to treatment groups, and surgery was performed at day 10 after tumor inoculation. For primary tumor resection, mice were anesthetized at 2% isoflurane, and the tumor was resected. Concurrently, a composition described herein comprising a polymer network (e.g., crosslinked hyaluronic acid) loaded with an innate immune response agonist (e.g., a RIG-1 agonist) was implanted at the tumor resection site. Empty polymer network was used as a negative control. Prolonged survival benefit was observed upon extended local release of RIG-1 agonist (FIG. 1). Survival curves were analyzed using Kaplan-Meier survival analysis with the log-rank (Mantel-Cox) test. Significant differences in survival were observed in animals treated with 7.5 μg of RIG-1 agonist when compared to negative control (p=0.0064).

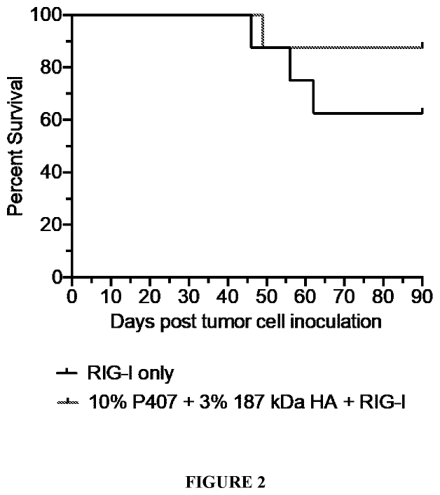

In one embodiment, in vivo animal studies were performed in 6-8 week old female BALB/cJ mice. Mice were inoculated orthotopically with 10⁵ 4T1-Luc2 breast cancer cells in their fourth mammary fat pad and tumors were allowed to grow. At day 10 after tumor inoculation, tumors were size-matched, mice were randomly assigned to treatment groups, and surgery was performed. For primary tumor resection, mice were anesthetized at 2% isoflurane, and the tumor was resected. Concurrently, a composition described herein comprising a polymer network (e.g., containing a poloxamer and hyaluronic acid hydrogel) loaded with an innate immune response agonist (e.g., a RIG-1 agonist, for example in some embodiments at a dose of 7.5 μg) was injected at the tumor resection site. As a negative control, RIG-1 agonist (e.g., in some embodiments at a dose of 7.5 μg) complexed with a polynucleotide agent carrier (e.g., in vivo-jetPEI) was directly injected into the tumor resection cavity. Prolonged survival benefit was observed upon extended local release of RIG-1 agonist (FIG. 2).

### Example 3. Preparation of an Exemplary Composition Comprised of a Nucleic Acid Encoding an Innate Immune Agonist Delivery Composition for Cancer Treatment

The present Example describes creation of a sustained release composition comprising a nucleic acid immunomodulatory agent suitable for implantation at a tumor resection site for cancer treatment. In some embodiments, such a nucleic acid immunomodulatory agent is or comprises an agonist of the innate immune system. In some embodiments the agonist of the innate immune system is encoded by a nucleic acid. In some embodiments, such a sustained release composition comprises at least one polymeric biomaterial and at least one nucleic acid, or at least one polymeric biomaterial, at least one nucleic acid and at least one nucleic acid carrier.

To prepare compositions described within some embodiments herein, nucleic acids encoding innate immune system agonists are complexed with a nucleic acid carrier (e.g., a transfection agent). In some embodiments, components are mixed in a volumetric ratio (e.g., a 1:1 ratio). In some embodiments the components are prepared in a sugar solution (e.g., a glucose solution). In some embodiments, the mixture is then incubated for a period of time.

To prepare compositions described within some embodiments herein, In vivo-jetPEI/IL-15 mRNA complexes were prepared by mixing 1:1 volume ratio of IL-15 mRNA (Pharna, #1304001) a 5% glucose solution with the in vivo-jetPEI reagent (N/P=6) in 5% glucose solution (PolyPlus, #201-10G). The mixture was then incubated at room temperature for 15 minutes before adding to the polymer network.

To prepare compositions according to some embodiments described herein, polymer networks are prepared using commercially available kits. In alternative embodiments described herein, polymer networks are prepared using unique polymer combinations not commercially available. In some embodiments, the polymeric biomaterial which has the potential to create a polymer network is placed within a mold. In some embodiments, the nucleic acid and nucleic acid carrier components are then added to the polymeric biomaterial. In some embodiments, chemical crosslinker is then added, and the product is allowed to incubate, creating a crosslinked polymer network. In some embodiments, chemical crosslinker is not required and gelation occurs during incubation under appropriate conditions (e.g., gelation enabling temperature) creating a suitable polymer network.

To prepare compositions according to some embodiments described herein, polymer networks were prepared using commercially available kits. In some embodiments, the polymeric biomaterial which has the potential to create a polymer network was placed within a mold. In some embodiments, the nucleic acid and nucleic acid carrier components were then added to the polymeric biomaterial. In some embodiments, chemical crosslinker was then added, and the product was allowed to incubate, creating a crosslinked polymer network.

To prepare compositions according to some embodiments described herein, polymer networks were prepared using the Hystem hydrogel kits (ESI Bio, GS1004). First, 120 μL of Glycosil (a crosslinkable hyaluronic acid e.g., a thiol-modified hyaluronic acid) was added into a Teflon mold (9 mm diameter), and then In vivo-jetPEI/IL-15 (2.5 μg, 7.5 μg, or 75 μg) was added, and 30 μL of chemical crosslinker (e.g., a thiol-reactive crosslinker such as polyethylene diacrylate, e.g., EXTRALINK® polyethylene glycol diacrylate cross-linker) was combined, the product was incubated for 1 hour to allow the polymer network to cross-link.

### Example 4. Use of Exemplary Nucleic Acid Delivery Compositions Comprised of a Nucleic Acid Encoding an Innate Immune Agonist for Cancer Treatment

The present Example describes administration of a sustained release composition comprising a nucleic acid immunomodulatory agent at a tumor resection site for cancer treatment. In some embodiments, such a nucleic acid immunomodulatory agent is or comprises an agonist of the innate immune system. In some embodiments the agonist of the innate immune system is encoded by a nucleic acid. In some embodiments, such a sustained release composition comprises at least one polymeric biomaterial and at least one nucleic acid, or at least one polymeric biomaterial, at least one nucleic acid and at least one nucleic acid carrier.

In some embodiments, in vivo studies utilized breast cancer cell lines grown in vitro. In some embodiments, prior to implantation, breast cancer cells were cultured in medium containing fetal bovine serum and a selective agent. In some embodiments, cells were cultured at physiological temperature, with known humidity, oxygen, and CO2 concentrations.

4T1-Luc2 breast cancer cells were cultured in RPMI-1640 medium, with 10% fetal bovine serum (FBS) and 1% penicillin/streptomycin. All cells were cultured in a 37° C. humidified incubator, with 5% CO2.

In some embodiments, in vivo animal studies are performed in mice. In some embodiments, mice are inoculated orthotopically with breast cancer cells in a mammary fat pad. In some embodiments, surgery is performed after a suitable time post tumor inoculation (e.g., at approximately day 10 after tumor inoculation). In some embodiments, concurrent to tumor excision, a composition as described herein comprising polymeric biomaterials or a polymer network loaded with a nucleic acid encoding an innate immune response agonist is administered at the tumor resection site. In some embodiments, a polymeric biomaterial or polymer network without a nucleic acid encoding an innate immune response agonist is administered at the tumor resection site to act as a relative negative control. In some embodiments, prolonged survival benefits are observed upon extended local release of a nucleic acid encoding an innate immune response agonist.

In some embodiments, in vivo animal studies were performed in mice. In some embodiments, mice were inoculated orthotopically with breast cancer cells in a mammary fat pad. In some embodiments, surgery was performed at day 10 after tumor inoculation. In some embodiments, concurrent to tumor excision, a composition as described herein comprising polymeric biomaterials or a polymer network loaded with a nucleic acid encoding an innate immune response agonist was administered at the tumor resection site. In some embodiments, a polymeric biomaterial or polymer network without a nucleic acid encoding an innate immune response agonist was administered at the tumor resection site to act as a relative negative control. In some embodiments, prolonged survival benefits were observed upon extended local release of a nucleic acid encoding an innate immune response agonist.

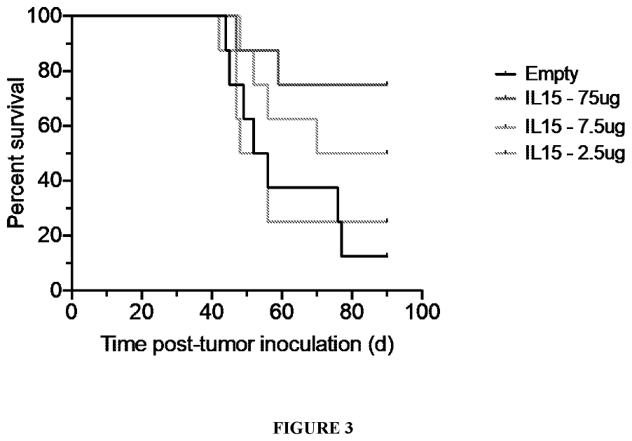

In some embodiments, in vivo animal studies were performed in 6-8 week old female BALB/cJ mice. Mice were inoculated orthotopically with 10⁵ 4T1-Luc2 breast cancer cells in their fourth mammary fat pad. Mice were size-matched and randomly assigned to treatment groups, and surgery was performed at day 10 after tumor inoculation. For primary tumor resection, mice were anesthetized at 2% isoflurane, and the tumor was resected. Concurrently, a composition described herein comprising a polymer network (e.g., crosslinked hyaluronic acid) loaded with an activator of the innate immune response (e.g., IL-15 mRNA) was implanted at the tumor resection site. Empty polymer network was used as a negative control. Prolonged survival benefit was observed upon extended local release of IL-15 mRNA (FIG. 3).

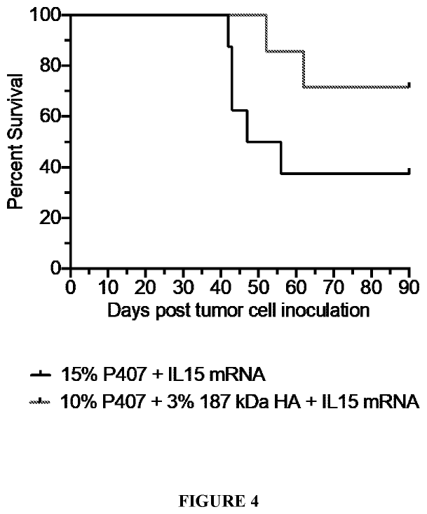

In one embodiment, in vivo animal studies were performed in 6-8 week old female BALB/cJ mice. Mice were inoculated orthotopically with 10⁵ 4T1-Luc2 breast cancer cells in their fourth mammary fat pad and tumors were allowed to grow. At day 10 after tumor inoculation, tumors were size-matched, mice were randomly assigned to treatment groups, and surgery was performed. For primary tumor resection, mice were anesthetized at 2% isoflurane, and the tumor was resected. Concurrently, a composition described herein comprising a polymer network (e.g., containing a poloxamer hydrogel or poloxamer and hyaluronic acid hydrogel) loaded with an activator of the innate immune response (e.g., IL-15 mRNA, for example in some embodiments at a dose of 75 μg) was injected at the tumor resection site. Prolonged survival benefit was observed upon treatment with compositions containing poloxamer and hyaluronic acid (FIG. 4). Without wishing to be bound by a particular theory, it is hypothesized that polymer networks containing poloxamer and hyaluronic acid result in enhanced survival relative to polymer networks containing poloxamer without hyaluronic acid due to prolonged extended release of the activator of the innate immune response (e.g., IL-15 mRNA) due to formation of longer polymer chains by hyaluronic acid than poloxamer and/or due to an immune stimulatory effect of hyaluronic acid.

### Example 5. Use and Immune Response Analysis of Exemplary Nucleic Acid Delivery Compositions Comprising an Immune Modulator for Cancer Treatment

The present Example relates to the collection and measurement of molecules associated with the immune system before, during, and after administration of a sustained release composition comprising a nucleic acid immunomodulatory agent as described herein at a tumor resection site for cancer treatment.

In some embodiments, in vivo studies utilize breast cancer cell lines grown in vitro. In some embodiments, prior to implantation, breast cancer cells are cultured in medium containing fetal bovine serum and a selective agent. In some embodiments, cells are cultured at physiological temperature, with known humidity, oxygen, and CO2 concentrations.

4T1-Luc2 breast cancer cells are cultured in RPMI-1640 medium, with 10% fetal bovine serum (FBS) and 1% penicillin/streptomycin. All cells are cultured in a 37° C. humidified incubator, with 5% CO2.

In some embodiments, in vivo animal studies are performed in mice. In some embodiments, prior to any additional treatments, blood draws are collected from mice in a manner suitable for continued longitudinal study as is known in the art, the blood is stored for later analysis as is known in the art. Mice are then inoculated orthotopically with breast cancer cells in a mammary fat pad. After a period of time suitable for solid tumor formation (e.g., approximately 6 days, 7 days, 8 days, and/or 9 days), blood draws are collected from mice in a manner suitable for continued longitudinal study as is known in the art, the blood is stored for later analysis as is known in the art. Tumor resection surgery is performed after a suitable time post tumor inoculation (e.g., at approximately day 10 after tumor inoculation). In some embodiments, concurrent to tumor excision, a composition as described herein comprising polymeric biomaterials or a polymer network loaded with a nucleic acid encoding an immune response modulator is administered at the tumor resection site. In some embodiments, a polymeric biomaterial or polymer network without a nucleic acid encoding an immune response modulator is administered at the tumor resection site to act as a relative negative control. At appropriate time points following tumor resection and biomaterial placement (e.g., approximately 1 week, 2 weeks, 3 weeks, 4 weeks, 6 weeks, 8 weeks, and 3 months) blood draws are collected from mice in a manner suitable for continued longitudinal study as is known in the art, the blood is stored for later analysis as is known in the art. Prolonged survival benefits are observed upon extended local release of a nucleic acid encoding an innate immune response agonist.

Following termination of the living portion of the example, blood samples are analyzed and measurements of molecules and cells associated with the immune system are conducted (e.g., Flow cytometry, ELISA, Western Blotting, and/or MS/MS). Measurements and levels of immune related molecules (e.g., cytokines such as IFN-α, IFN-β, etc.) and circulating immune cell subsets are compared for correlation with survival. Animals showing prolonged survival exhibit optimal levels of circulating immune cells and immune related molecules.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

The entire content of International Patent Publication No. WO 2018/045058 (e.g., compositions, devices, methods of preparation, methods of use, and kits) is incorporated herein by reference for the purposes described herein.

What is claimed is:

1. A method comprising a step of:
administering to a target site in a tumor resection subject, a composition comprising:
(i) a polymeric biomaterial; and
(ii) a polynucleotide agent that encodes or regulates an immunomodulatory polypeptide.

2. The method of claim 1, wherein the polynucleotide agent encodes a cytokine that induces innate immunity and/or adaptive immunity.

3. The method of claim 1, wherein the polynucleotide agent activates a pattern recognition receptor that induces innate immunity.

4. The method of claim 1, wherein the polynucleotide agent encodes a chemokine that induces recruitment of immune cells.

5. The method of claim 1, wherein the polynucleotide agent encodes an antibody that mediates immune checkpoint blockade or co-stimulation.

6. The method of any one of claims 1-5, wherein the target site is the tumor resection site.

7. The method of any one of claims 1-5, wherein the target site is a site near the tumor resection site.

8. The method of any one of claims 1-5, wherein the target site is a sentinel lymph node.

9. The method of any one of claims 1-8, wherein the step of administering is by injection.

10. The method of claim 9, wherein the composition is liquid and the polymeric biomaterial is a viscous polymer solution.

11. The method of claim 9, wherein the composition is liquid and the polymeric biomaterial, upon the administration, forms a polymer network biomaterial in situ at the target site.

12. The method of claim 11, wherein the polymer network biomaterial comprises or is a crosslinked polymer network biomaterial.

13. The method of claim 11, wherein the polymer network biomaterial comprises or is a non-crosslinked polymer network biomaterial.

14. The method of any one of claims 1-13, wherein the polymeric biomaterial is characterized in that when tested in vitro by placing a combination of the polymeric biomaterial and the polynucleotide agent in PBS (pH 7.4), less than 100% of the polynucleotide agent is released within 3 hours from the polymeric biomaterial.

15. The method of any one of claims 1-13, wherein the polymeric biomaterial is characterized in that when tested in vivo by administering a combination of the polymeric biomaterial and the polynucleotide agent to a mammary fat pad of a mouse subject, less than or equal to 50% of the polynucleotide agent is released in vivo 8 hours after the administration.

16. The method of any one of claims 1-13, wherein the polymeric biomaterial is characterized in that when tested in vivo by administering a combination of the polymeric biomaterial and the polynucleotide agent to a mammary fat pad of a mouse subject, the polymeric biomaterial extends release of the polynucleotide agent such that, when assessed at 24 hours after administration, more polynucleotide agent is present at the mammary fat pad than is observed when the polynucleotide agent is administered in solution.

17. The method of any one of claims 1-13, wherein the polymeric biomaterial is characterized in that the polynucleotide agent is released from the polymeric biomaterial and is taken up by local cells so that (i) at least a subset of local immune cells express the immunomodulatory polypeptide encoded by the polynucleotide agent, (ii) at least a subset of local immune cells have an increased expression of a type 1 interferon in response to innate immune stimulation induced by the polynucleotide agent, and/or (iii) at least a subset of local immune cells have a change in level and/or activity of the immunomodulatory polypeptide.

18. The method of any one of claims 1-17, wherein the polymeric biomaterial is characterized by a storage modulus of about 10 Pa to about 5,000 Pa.

19. The method of any one of claims 1-18, wherein the polymeric biomaterial comprises or is a hydrogel.

20. The method of any one of claims 1-18, wherein the polymeric biomaterial comprises a positively-charged polymer.

21. The method of any one of claims 1-20, wherein the polymeric biomaterial further comprises a polynucleotide agent carrier.

22. The method of claim 21, wherein the polynucleotide agent is complexed to the polynucleotide agent carrier.

23. The method of claim 21, wherein the polynucleotide agent is loaded in the polynucleotide agent carrier.

24. The method of any one of claims 21-23, wherein the polynucleotide agent carrier comprises a cationic agent and/or a lipid.

25. The method of any one of claims 1-24, wherein the step of administering does not involve administration of a tumor antigen to the tumor resection subject.

26. The method of any one of claims 1-25, wherein the step of administering does not involve administration of a microparticle to the tumor resection subject.

27. The method of any one of claims 1-26, wherein the step of administering does not involve adoptive transfer of immune cells to the tumor resection subject.

28. The method of any one of claims 1-27, wherein the composition further comprises an inhibitor of proinflammatory immune response mediated by p38 mitogen-activated protein kinase (MAPK) pathway.

29. The method of any one of claims 1-28, wherein the composition further comprises an activator of innate immunity.

30. The method of claim 29, wherein the activator of innate immunity is or comprises a stimulator of interferon genes (STING) agonist.

31. The method of claim 29, wherein the activator of innate immunity is or comprises a Toll-like receptor (TLR) 7 and/or TLR8 (“TLR7/8”) agonist.

32. The method of any one of claims 1-31, wherein the composition further comprises an activator of adaptive immunity.

33. The method of any one of claims 1-32, wherein the polymeric biomaterial forms a matrix or depot and the polynucleotide agent is within the polymeric biomaterial.

34. The method of claim 33, wherein the polynucleotide agent is released by diffusion through the polymeric biomaterial.

35. The method of any one of claims 1-34, wherein the polymeric biomaterial is biodegradable in vivo.

36. The method of any one of claims 1-35, wherein the tumor resection site is characterized by absence of gross residual tumor antigen.

37. The method of any one of claims 1-36, wherein the tumor resection subject is suffering from metastatic cancer.

38. The method of claim 37, further comprising a step of monitoring at least one metastatic site in the tumor resection subject after the administration.

39. A method of generating a polymer network biomaterial composition, the method comprising steps of:
(a) providing a composition comprising one or more precursor components of a polymer network biomaterial and a polynucleotide agent that encodes or regulates an immunomodulatory polypeptide; and
(b) allowing the precursor components to form a polymer network biomaterial in less than 10 minutes, wherein the polymer network biomaterial is characterized in that:
the polymer network biomaterial has a storage modulus of less than 5,000 Pa; and/or
when tested in vitro by placing a combination of the polymer network biomaterial and the polynucleotide agent in PBS (pH 7.4), less than 100% of the polynucleotide agent is released within 3 hours from the polymer network biomaterial;
when tested in vivo by administering a combination of the polymer network biomaterial and the polynucleotide agent to a mammary fat pad of a mouse subject, less than or equal to 50% of the polynucleotide agent is released in vivo 8 hours after the administration;
when tested in vivo by administering a combination of the polymer network biomaterial and the polynucleotide agent to a mammary fat pad of a mouse subject, the polymer network biomaterial extends release of the polynucleotide agent such that, when assessed at 24 hours after administration, more polynucleotide agent is present at the mammary fat pad than is observed when the polynucleotide agent is administered in solution; and/or
the polynucleotide agent released from the polymer network biomaterial is taken up by local cells so that (i) at least a subset of local immune cells express the immunomodulatory polypeptide encoded by the polynucleotide agent, (ii) at least a subset of local immune cells have an increased expression of a type 1 interferon in response to innate immune stimulation induced by the polynucleotide agent, and/or (iii) at least a subset of local immune cells have a change in level and/or activity of the immunomodulatory polypeptide.

40. The method of claim 39, wherein the polymer network biomaterial comprises or is a crosslinked polymer network biomaterial.

41. The method of claim 39, wherein the polymer network biomaterial comprises or is a non-crosslinked polymer network biomaterial.

42. The method of any one of claims 39-41, wherein the polymer network biomaterial comprises or is a hydrogel.

43. The method of any one of claims 39-42, wherein the polymer network biomaterial comprises a polynucleotide agent carrier.

44. The method of claim 43, wherein the polynucleotide agent is complexed to the polynucleotide agent carrier.

45. The method of claim 43, wherein the polynucleotide agent is loaded in the polynucleotide agent carrier.

46. The method of any one of claims 43-45, wherein the polynucleotide agent carrier comprises a cationic agent and/or a lipid.

47. A method of characterizing a polymer network biomaterial composition or component(s) thereof, the method comprising steps of:
(a) providing a polymer network biomaterial composition in a buffered solution in vitro, wherein the polymer network biomaterial composition comprises a candidate polymeric biomaterial and a polynucleotide agent that encodes or regulates an immunomodulatory polypeptide; and
(b) determining whether less than 100% of the polynucleotide agent is released within 3 hours from the polymeric biomaterial.

48. The method of claim 47, wherein the buffered solution comprises or is PBS (pH 7.4).

49. The method of claim 47 or 48, wherein the step of determining further comprises measuring amounts of the polynucleotide agent released from the polymeric biomaterial at pre-determined time points over a period of at least 3 hours.

50. The method of claim 49, wherein the step of determining further comprises determining a release profile kinetics of the polynucleotide agent from the polymeric biomaterial.

51. The method of any one of claims 47-50, further comprising selecting a polymer network biomaterial composition characterized in that less than 100% of the polynucleotide agent is released within 3 hours from the polymeric biomaterial.

52. The method of claim 51, further comprising contacting the selected polymer network biomaterial composition with a population of cells, and determining:
(a) whether the polynucleotide agent released from the candidate polymeric biomaterial is taken up by immune cells; and/or
(b) whether the immune cells that take up the polynucleotide agent exhibits at least one of the following biological activities:
(i) expressing the immunomodulatory polypeptide encoded by the polynucleotide agent,
(ii) exhibiting an increased expression of a type 1 interferon in response to innate immune stimulation induced by the polynucleotide agent, and/or
(iii) exhibiting a change in level and/or activity of the immunomodulatory polypeptide.

53. The method of claim 52, wherein the immune cells comprise myeloid cells and/or plasmacytoid dendritic cells.

54. The method of claim 52, wherein the cells further comprise non-immune cells.

55. The method of claim 54, wherein the non-immune cells comprise fibroblasts and/or endothelial cells.

56. The method of claim 54 or 55, further comprising determining:
(a) whether the polynucleotide agent released from the candidate polymeric biomaterial is taken up by non-immune cells; and/or
(b) whether the polynucleotide agent exerts an adverse effect on the non-immune cells.

57. A method of characterizing a polymer network biomaterial composition or component(s) thereof, the method comprising steps of:
(a) administering a polymer network composition to a target site of a mouse subject in vivo, wherein the polymer network biomaterial composition comprises a candidate polymeric biomaterial and a polynucleotide agent that encodes or regulates an immunomodulatory polypeptide; and
(b) determining:
(i) whether the candidate polymeric biomaterial extends release of the polynucleotide agent at the target site, upon administration, by at least 24 hours relative to administration of the same polynucleotide agent in solution without the polymeric biomaterial; and/or
(ii) whether release profile of the polynucleotide agent from the candidate polymeric biomaterial is characterized in that: less than or equal to 50% of the polynucleotide agent is released in vivo 8 hours after the administration.

58. The method of claim 57, wherein the target site is a mammary fat pad.

59. The method of claim 57 or 58, further comprising selecting a polymer network biomaterial composition characterized in that (i) the candidate polymeric biomaterial extends release of the polynucleotide agent at the target site, upon administration, by at least 24 hours relative to administration of the same polynucleotide agent in solution without the polymeric biomaterial; and/or (ii) less than or equal to 50% of the polynucleotide agent is released from the candidate polymeric biomaterial in vivo 8 hours after the administration.

60. The method of any one of claims 57-59, further comprising determining:
(a) whether the polynucleotide agent released from the candidate polymeric biomaterial is taken up by immune cells; and/or
(b) whether the immune cells that take up the polynucleotide agent exhibits at least one of the following biological activities:
(i) expressing the immunomodulatory polypeptide encoded by the polynucleotide agent,
(ii) exhibiting an increased expression of a type 1 interferon in response to innate immune stimulation induced by the polynucleotide agent, and/or
(ii) exhibiting a change in level and/or activity of the immunomodulatory polypeptide.

61. The method of claim 60, further comprising determining:
(c) whether cellular uptake of the polynucleotide agent released from the candidate polymeric biomaterial is delayed, as compared to that when the cells are contacted with the polynucleotide agent in the absence of the candidate polymeric biomaterial; and/or
(d) whether at least one of the biological activities induced by the polynucleotide agent is delayed, as compared to that when the cells are contacted with the polynucleotide agent in the absence of the candidate polymeric biomaterial.

62. The method of claim 60 or 61, wherein the immune cells comprise myeloid cells and/or plasmacytoid dendritic cells.

63. The method of any one of claims 60-62, further comprising determining:
(e) whether the polynucleotide agent released from the candidate polymeric biomaterial is taken up by non-immune cells; and/or
(f) whether the polynucleotide agent exerts an adverse effect on the non-immune cells.

64. The method of claim 63, wherein the non-immune cells comprise fibroblasts and/or endothelial cells.

65. A method comprising a step of:
administering to a target site in a tumor resection subject, a composition comprising:
(i) a polynucleotide agent carrier; and
(ii) a polynucleotide agent that encodes or regulates an immunomodulatory polypeptide.

66. The method of claim 65, wherein the polynucleotide agent carrier comprises a cationic agent and/or a lipid.
